(19) United States
(12) Patent Application Publication
(10) Pub. No.: US 2022/0101113 A1
(43) Pub. Date: Mar. 31, 2022

(54) KNOWLEDGE DISCOVERY USING A NEURAL NETWORK

(71) Applicant: NVIDIA Corporation, Santa Clara, CA (US)

(72) Inventors: Lickkong Tam, Santa Clara, CA (US); Xiaosong Wang, Rockville, MD (US); Daguang Xu, Potomac, MD (US)

(21) Appl. No.: 17/033,439

(22) Filed: Sep. 25, 2020

(51) Int. Cl. G06N 3/08; G06N 3/04; G06K 9/62; G06F 40/263; G06F 40/284; G16H 70/40

(52) U.S. Cl. CPC G06N 3/08; G06N 3/04; G16H 70/40; G06F 40/263; G06F 40/284; G06K 9/6256

(57) ABSTRACT

Apparatuses, systems, and techniques to identify one or more relationships among one or more words using one or more transformer-based language neural networks trained using domain-specific data.

### TECHNICAL FIELD

At least one embodiment pertains to processing resources used to perform and facilitate artificial intelligence. For example, at least one embodiment pertains to processors or computing systems used to train and use neural networks according to various novel techniques described herein.

### BACKGROUND

Knowledge discovery in a large literature corpus is difficult, especially for accessing latent domain-specific knowledge within a large literature corpus. For example, it is difficult, even for experts in a specific field, to discover a property of a particular drug (e.g., efficacy of a drug in clinical trials) in a large literature corpus. A traditional solution uses a skip-gram word2vec language modelling method. This solution is based on exact word mappings of words to vectors in a vector space and does not take into account negation, flexible phrases, or other context in which these words are used, thereby not achieving high accuracy in modelling.

### BRIEF DESCRIPTION OF DRAWINGS

FIG. 1A illustrates inference and/or training logic, according to at least one embodiment;

FIG. 1B illustrates inference and/or training logic, according to at least one embodiment;

FIG. 2 illustrates training and deployment of a neural network, according to at least one embodiment;

FIG. 3 is an example data flow diagram for a process to train one or more transformer-based language neural networks using domain-specific data, in accordance with at least one embodiment;

FIG. 4 is an example attention visualization of statistical properties of relationships of words in domain-specific data for masked language predictions, in accordance with at least one embodiment;

FIG. 5 is an example data flow diagram for a process to use one or more transformer-based language neural networks to identify one or more relationships among one or more words, in accordance with at least one embodiment;

FIG. 6 is an example data flow diagram for a process to use one or more transformer-based language neural networks, which are trained using domain-specific data, to identify one or more drugs described in one or more documents, in accordance with at least one embodiment;

FIG. 7 is a table showing a summary of a sample clinical trials dataset, in accordance with at least one embodiment;

FIG. 8 is an example graph illustrating a number of candidate drugs of a specific type trialed each year and a total of specific-type drugs granted FDA approval over time, in accordance with at least one embodiment;

FIG. 9 is an example table of analogy categories used for evaluation of semantic learning, in accordance with at least one embodiment;

FIG. 10 is an example attention visualization of statistical properties of relationships of a query word in domain-specific data and target words of a target phrase, in accordance with at least one embodiment;

FIG. 11 is an example passage in domain-specific data that is highlighted on a per-sentence basis using a target term, in accordance with at least one embodiment;

FIG. 12 is an example graph illustrating a prediction rank of drug candidates over years and indications of FDA approval, in accordance with at least one embodiment;

FIG. 13 is an example graph illustrating a ranking by confidence score of COVID-19 efficacy for on-going clinical trials, in accordance with at least one embodiment;

FIG. 14 is an example graph illustrating analogy mining for a drug to clinical trials efficacy, in accordance with at least one embodiment;

FIG. 15 is a flow diagram of a process to identify one or more relationships among one or more words using one or more transformer-based language neural networks trained using domain-specific data, in accordance with at least one embodiment;

FIG. 16 is a flow diagram of a process to identify one or more drugs described in one or more documents using a transformer-based language neural network that is trained using domain-specific data, in accordance with at least one embodiment;

FIG. 17 is a block diagram illustrating a computer system, according to at least one embodiment;

FIG. 18 is a block diagram illustrating an exemplary computer system, which may be a system with interconnected devices and components, a system-on-a-chip (SOC) or some combination thereof formed with a processor that may include execution units to execute an instruction, according to at least one embodiment;

FIG. 19 is a block diagram illustrating an electronic device for utilizing a processor, according to at least one embodiment.

FIG. 20 illustrates a computer system, according to at least one embodiment;

FIG. 21 illustrates a computer system, according to at least one embodiment;

FIG. 22A illustrates a computer system, according to at least one embodiment;

FIG. 22B illustrates a computer system, according to at least one embodiment;

FIG. 22C illustrates a computer system, according to at least one embodiment;

FIG. 22D illustrates a computer system, according to at least one embodiment;

FIGS. 22E and 22F illustrate a shared programming model, according to at least one embodiment;

FIG. 23 is a block diagram illustrating an exemplary system on a chip integrated circuit that may be fabricated using one or more IP cores, according to at least one embodiment;

FIGS. 24A-24B illustrate exemplary integrated circuits and associated graphics processors, according to at least one embodiment;

FIGS. 25A-25B illustrate additional exemplary graphics processor logic according to at least one embodiment;

FIG. 26 illustrates a computer system, according to at least one embodiment;

FIG. 27A illustrates a parallel processor, according to at least one embodiment;

FIG. 27B illustrates a partition unit, according to at least one embodiment;

FIG. 27C illustrates a processing cluster, according to at least one embodiment;

FIG. 27D illustrates a graphics multiprocessor, according to at least one embodiment;

FIG. 28 illustrates a multi-graphics processing unit (GPU) system, according to at least one embodiment;

FIG. 29 illustrates a graphics processor, according to at least one embodiment;

FIG. 30 is a block diagram illustrating a processor micro-architecture for a processor, according to at least one embodiment;

FIG. 31 illustrates a deep learning application processor, according to at least one embodiment;

FIG. 32 is a block diagram illustrating an example neuromorphic processor, according to at least one embodiment;

FIG. 33 illustrates at least portions of a graphics processor, according to one or more embodiments;

FIG. 34 illustrates at least portions of a graphics processor, according to one or more embodiments;

FIG. 35 illustrates at least portions of a graphics processor, according to one or more embodiments;

FIG. 36 is a block diagram of a graphics processing engine of a graphics processor in accordance with at least one embodiment;

FIG. 37 is a block diagram of at least portions of a graphics processor core, according to at least one embodiment;

FIGS. 38A-38B illustrate thread execution logic including an array of processing elements of a graphics processor core according to at least one embodiment;

FIG. 39 illustrates a parallel processing unit (“PPU”), according to at least one embodiment;

FIG. 40 illustrates a general processing cluster (“GPC”), according to at least one embodiment;

FIG. 41 illustrates a memory partition unit of a parallel processing unit (“PPU”), according to at least one embodiment;

FIG. 42 illustrates a streaming multi-processor, according to at least one embodiment.

FIG. 43 is an example data flow diagram for an advanced computing pipeline, in accordance with at least one embodiment;

FIG. 44 is a system diagram for an example system for training, adapting, instantiating and deploying machine learning models in an advanced computing pipeline, in accordance with at least one embodiment.

### DETAILED DESCRIPTION

### Inference and Training Logic

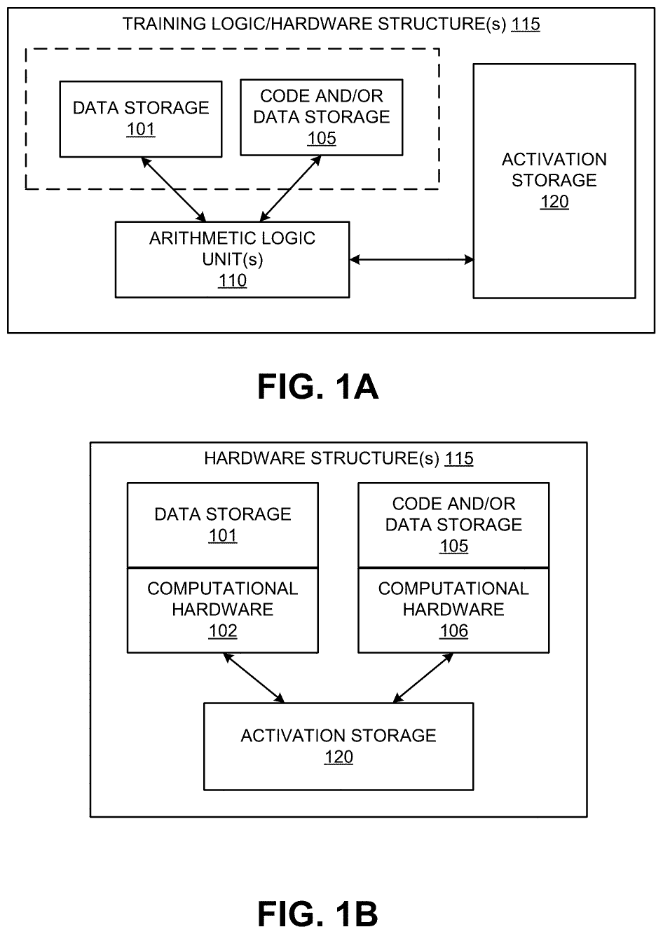

FIG. 1A illustrates inference and/or training logic 115 used to perform inferencing and/or training operations associated with one or more embodiments. Details regarding inference and/or training logic 115 are provided below in conjunction with FIGS. 1A and/or 1B.

In at least one embodiment, inference and/or training logic 115 may include, without limitation, code and/or data storage 101 to store forward and/or output weight and/or input/output data, and/or other parameters to configure neurons or layers of a neural network trained and/or used for inferencing in aspects of one or more embodiments. In at least one embodiment, training logic 115 may include, or be coupled to code and/or data storage 101 to store graph code or other software to control timing and/or order, in which weight and/or other parameter information is to be loaded to configure, logic, including integer and/or floating point units (collectively, arithmetic logic units (ALUs) or simply circuits). In at least one embodiment, code, such as graph code, loads weight or other parameter information into processor ALUs based on an architecture of a neural network to which such code corresponds. In at least one embodiment, code and/or data storage 101 stores weight parameters and/or input/output data of each layer of a neural network trained or used in conjunction with one or more embodiments during forward propagation of input/output data and/or weight parameters during training and/or inferencing using aspects of one or more embodiments. In at least one embodiment, any portion of code and/or data storage 101 may be included with other on-chip or off-chip data storage, including a processor's L1, L2, or L3 cache or system memory.

In at least one embodiment, any portion of code and/or data storage 101 may be internal or external to one or more processors or other hardware logic devices or circuits. In at least one embodiment, code and/or code and/or data storage 101 may be cache memory, dynamic randomly addressable memory (“DRAM”), static randomly addressable memory (“SRAM”), non-volatile memory (e.g., flash memory), or other storage. In at least one embodiment, a choice of whether code and/or code and/or data storage 101 is internal or external to a processor, for example, or comprising DRAM, SRAM, flash or some other storage type may depend on available storage on-chip versus off-chip, latency requirements of training and/or inferencing functions being performed, batch size of data used in inferencing and/or training of a neural network, or some combination of these factors.

In at least one embodiment, inference and/or training logic 115 may include, without limitation, a code and/or data storage 105 to store backward and/or output weight and/or input/output data corresponding to neurons or layers of a neural network trained and/or used for inferencing in aspects of one or more embodiments. In at least one embodiment, code and/or data storage 105 stores weight parameters and/or input/output data of each layer of a neural network trained or used in conjunction with one or more embodiments during backward propagation of input/output data and/or weight parameters during training and/or inferencing using aspects of one or more embodiments. In at least one embodiment, training logic 115 may include, or be coupled to code and/or data storage 105 to store graph code or other software to control timing and/or order, in which weight and/or other parameter information is to be loaded to configure, logic, including integer and/or floating point units (collectively, arithmetic logic units (ALUs).

In at least one embodiment, code, such as graph code, causes a loading of weight or other parameter information into processor ALUs based on an architecture of a neural network to which such code corresponds. In at least one embodiment, any portion of code and/or data storage 105 may be included with other on-chip or off-chip data storage, including a processor's L1, L2, or L3 cache or system memory. In at least one embodiment, any portion of code and/or data storage 105 may be internal or external to one or more processors or other hardware logic devices or circuits. In at least one embodiment, code and/or data storage 105 may be cache memory, DRAM, SRAM, non-volatile memory (e.g., flash memory), or other storage. In at least one embodiment, a choice of whether code and/or data storage 105 is internal or external to a processor, for example, or comprising DRAM, SRAM, flash memory or some other storage type may depend on available storage on-chip versus off-chip, latency requirements of training and/or inferencing functions being performed, batch size of data used in inferencing and/or training of a neural network, or some combination of these factors.

In at least one embodiment, code and/or data storage 101 and code and/or data storage 105 may be separate storage structures. In at least one embodiment, code and/or data storage 101 and code and/or data storage 105 may be a combined storage structure. In at least one embodiment, code and/or data storage 101 and code and/or data storage 105 may be partially combined and partially separate. In at least one embodiment, any portion of code and/or data storage 101 and code and/or data storage 105 may be included with other on-chip or off-chip data storage, including a processor's L1, L2, or L3 cache or system memory.

In at least one embodiment, inference and/or training logic 115 may include, without limitation, one or more arithmetic logic unit(s) (“ALU(s)”) 110, including integer and/or floating point units, to perform logical and/or mathematical operations based, at least in part on, or indicated by, training and/or inference code (e.g., graph code), a result of which may produce activations (e.g., output values from layers or neurons within a neural network) stored in an activation storage 120 that are functions of input/output and/or weight parameter data stored in code and/or data storage 101 and/or code and/or data storage 105. In at least one embodiment, activations stored in activation storage 120 are generated according to linear algebraic and or matrix-based mathematics performed by ALU(s) 110 in response to performing instructions or other code, wherein weight values stored in code and/or data storage 105 and/or data storage 101 are used as operands along with other values, such as bias values, gradient information, momentum values, or other parameters or hyperparameters, any or all of which may be stored in code and/or data storage 105 or code and/or data storage 101 or another storage on or off-chip.

In at least one embodiment, ALU(s) 110 are included within one or more processors or other hardware logic devices or circuits, whereas in at least one other embodiment, ALU(s) 110 may be external to a processor or other hardware logic device or circuit that uses them (e.g., a co-processor). In at least one embodiment, ALUs 110 may be included within a processor's execution units or otherwise within a bank of ALUs accessible by a processor's execution units either within same processor or distributed between different processors of different types (e.g., central processing units, graphics processing units, fixed function units, etc.). In at least one embodiment, code and/or data storage 101, code and/or data storage 105, and activation storage 120 may share a processor or other hardware logic device or circuit, whereas in at least one other embodiment, they may be in different processors or other hardware logic devices or circuits, or some combination of same and different processors or other hardware logic devices or circuits. In at least one embodiment, any portion of activation storage 120 may be included with other on-chip or off-chip data storage, including a processor's L1, L2, or L3 cache or system memory. Furthermore, inferencing and/or training code may be stored with other code accessible to a processor or other hardware logic or circuit and fetched and/or processed using a processor's fetch, decode, scheduling, execution, retirement and/or other logical circuits.

In at least one embodiment, activation storage 120 may be cache memory, DRAM, SRAM, non-volatile memory (e.g., flash memory), or other storage. In at least one embodiment, activation storage 120 may be completely or partially within or external to one or more processors or other logical circuits. In at least one embodiment, a choice of whether activation storage 120 is internal or external to a processor, for example, or comprising DRAM, SRAM, flash memory or some other storage type may depend on available storage on-chip versus off-chip, latency requirements of training and/or inferencing functions being performed, batch size of data used in inferencing and/or training of a neural network, or some combination of these factors.

In at least one embodiment, inference and/or training logic 115 illustrated in FIG. 1A may be used in conjunction with an application-specific integrated circuit (“ASIC”), such as a TensorFlow® Processing Unit from Google, an inference processing unit (IPU) from Graphcore™, or a Nervana® (e.g., “Lake Crest”) processor from Intel Corp. In at least one embodiment, inference and/or training logic 115 illustrated in FIG. 1A may be used in conjunction with central processing unit (“CPU”) hardware, graphics processing unit (“GPU”) hardware or other hardware, such as field programmable gate arrays (“FPGAs”).

FIG. 1B illustrates inference and/or training logic 115, according to at least one embodiment. In at least one embodiment, inference and/or training logic 115 may include, without limitation, hardware logic in which computational resources are dedicated or otherwise exclusively used in conjunction with weight values or other information corresponding to one or more layers of neurons within a neural network. In at least one embodiment, inference and/or training logic 115 illustrated in FIG. 1B may be used in conjunction with an application-specific integrated circuit (ASIC), such as TensorFlow® Processing Unit from Google, an inference processing unit (IPU) from Graphcore™, or a Nervana® (e.g., “Lake Crest”) processor from Intel Corp. In at least one embodiment, inference and/or training logic 115 illustrated in FIG. 1B may be used in conjunction with central processing unit (CPU) hardware, graphics processing unit (GPU) hardware or other hardware, such as field programmable gate arrays (FPGAs). In at least one embodiment, inference and/or training logic 115 includes, without limitation, code and/or data storage 101 and code and/or data storage 105, which may be used to store code (e.g., graph code), weight values and/or other information, including bias values, gradient information, momentum values, and/or other parameter or hyperparameter information. In at least one embodiment illustrated in FIG. 1B, each of code and/or data storage 101 and code and/or data storage 105 is associated with a dedicated computational resource, such as computational hardware 102 and computational hardware 106, respectively. In at least one embodiment, each of computational hardware 102 and computational hardware 106 comprises one or more ALUs that perform mathematical functions, such as linear algebraic functions, only on information stored in code and/or data storage 101 and code and/or data storage 105, respectively, result of which is stored in activation storage 120.

In at least one embodiment, each of code and/or data storage 101 and 105 and corresponding computational hardware 102 and 106, respectively, correspond to different layers of a neural network, such that resulting activation from one storage/computational pair 101/102 of code and/or data storage 101 and computational hardware 102 is provided as an input to a next storage/computational pair 105/106 of code and/or data storage 105 and computational hardware 106, in order to mirror a conceptual organization of a neural network. In at least one embodiment, each of storage/computational pairs 101/102 and 105/106 may correspond to more than one neural network layer. In at least one embodiment, additional storage/computation pairs (not shown) subsequent to or in parallel with storage/computation pairs 101/102 and 105/106 may be included in inference and/or training logic 115.

### Neural Network Training and Deployment

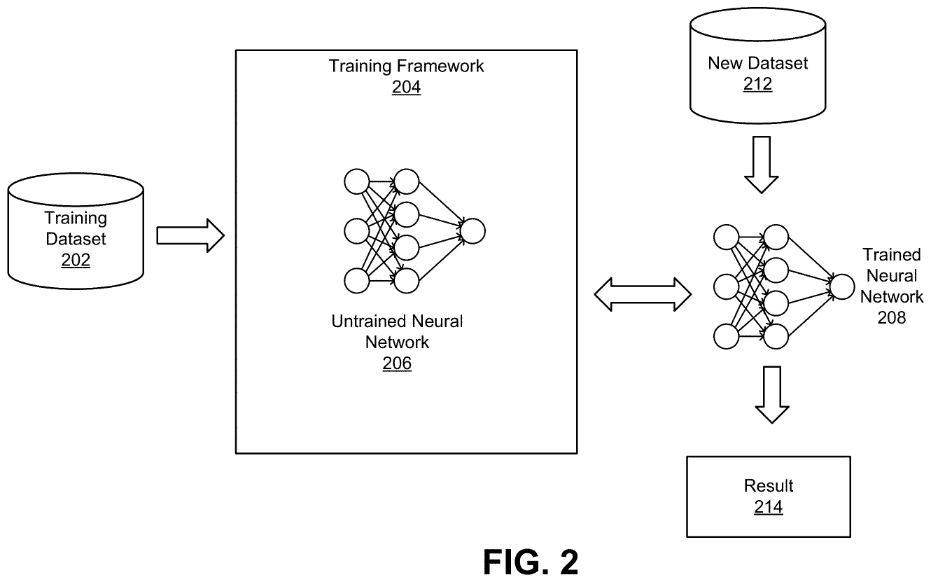

FIG. 2 illustrates training and deployment of a deep neural network, according to at least one embodiment. In at least one embodiment, untrained neural network 206 is trained using a training dataset 202. In at least one embodiment, training framework 204 is a PyTorch framework, whereas in other embodiments, training framework 204 is a TensorFlow, Boost, Caffe, Microsoft Cognitive Toolkit/CNTK, MXNet, Chainer, Keras, Deeplearning4j, or other training framework. In at least one embodiment, training framework 204 trains an untrained neural network 206 and enables it to be trained using processing resources described herein to generate a trained neural network 208. In at least one embodiment, weights may be chosen randomly or by pre-training using a deep belief network. In at least one embodiment, training may be performed in either a supervised, partially supervised, or unsupervised manner.

In at least one embodiment, untrained neural network 206 is trained using supervised learning, wherein training dataset 202 includes an input paired with a desired output for an input, or where training dataset 202 includes input having a known output and an output of neural network 206 is manually graded. In at least one embodiment, untrained neural network 206 is trained in a supervised manner and processes inputs from training dataset 202 and compares resulting outputs against a set of expected or desired outputs. In at least one embodiment, errors are then propagated back through untrained neural network 206. In at least one embodiment, training framework 204 adjusts weights that control untrained neural network 206. In at least one embodiment, training framework 204 includes tools to monitor how well untrained neural network 206 is converging towards a model, such as trained neural network 208, suitable to generating correct answers, such as in result 214, based on input data such as a new dataset 212. In at least one embodiment, training framework 204 trains untrained neural network 206 repeatedly while adjust weights to refine an output of untrained neural network 206 using a loss function and adjustment algorithm, such as stochastic gradient descent. In at least one embodiment, training framework 204 trains untrained neural network 206 until untrained neural network 206 achieves a desired accuracy. In at least one embodiment, trained neural network 208 can then be deployed to implement any number of machine learning operations.

In at least one embodiment, untrained neural network 206 is trained using unsupervised learning, wherein untrained neural network 206 attempts to train itself using unlabeled data. In at least one embodiment, unsupervised learning training dataset 202 will include input data without any associated output data or “ground truth” data. In at least one embodiment, untrained neural network 206 can learn groupings within training dataset 202 and can determine how individual inputs are related to untrained dataset 202. In at least one embodiment, unsupervised training can be used to generate a self-organizing map in trained neural network 208 capable of performing operations useful in reducing dimensionality of new dataset 212. In at least one embodiment, unsupervised training can also be used to perform anomaly detection, which allows identification of data points in new dataset 212 that deviate from normal patterns of new dataset 212.

In at least one embodiment, semi-supervised learning may be used, which is a technique in which in training dataset 202 includes a mix of labeled and unlabeled data. In at least one embodiment, training framework 204 may be used to perform incremental learning, such as through transferred learning techniques. In at least one embodiment, incremental learning enables trained neural network 208 to adapt to new dataset 212 without forgetting knowledge instilled within trained neural network 208 during initial training.

### Transformer-Based Language Neural Networks for Domain-Specific Knowledge Discovery

As described above, it is difficult accessing latent domain-specific knowledge within a large literature corpus. For example, it is difficult, even for experts in biochemistry and organic chemistry, to discovery a property of a particular drug (e.g., efficacy of a drug in clinical trials or whether a drug is an inhibitor) in a large literature corpus. Some language modeling methods, such as a skip-gram word2vec language modelling method, have been used to access latent domain-specific knowledge. Skip-gram word2vec language modelling method, which is based on exact word mappings, does not take into account negation or other context in which these words are used, thereby not achieving high accuracy in language modelling. Given a requirement for exact word matching and an amount of domain-specific terminology used in a large domain-specific dataset, a skip-gram word2vec model suffers from a vocabulary problem and a resulting computational problem during an inference phase.

In at least one embodiment, one or more transformer-based language neural networks are used to be able to predict statistical properties of word phrases present in a large literature corpus based on word context and to predict associations between different phrases present in a literature corpus. In at least one embodiment, a known transformer-based language neural network, such as a Bidirectional Encoder Representations from Transformers (BERT) neural network, is modified to produce one or more transformer-based language neural networks that can predict statistical properties of word phrases and their associations present in a large literature corpus. In at least one embodiment, a transformer-based language neural network is trained using a Robustly Optimized Bidirectional Encoder Representations from Transformers Approach (RoBERTa) and domain-specific data, as opposed to a large literature corpus that is domain agnostic. RoBERTa uses a BERT model while modifying key hyper-parameters of BERT model and removing next-sentence detection objective when training BERT model. In at least one embodiment, a RoBERTa-based neural network uses byte-level encoding, referred to as byte-pair encoding (BPE), as a tokenizer and uses a different pre-training scheme than a BERT neural network. In at least one embodiment, a RoBERTa language neural network is trained on well-curated, domain-specific datasets to isolate and extract relevant information. In at least one embodiment, domain-specific datasets include one or more documents that are specific to a particular subject. In at least one embodiment, a RoBERTa-based neural network (also referred to as a RoBERTa-large model) is trained on a large corpus of domain-specific data and is modified, during inference, to compute query-target (QT) predictions for identifying relationships or associations between query phrases and respective target phrases.

In at least one embodiment, one or more transformer-based language neural networks (e.g., RoBERTa-based neural network modified as discussed above) can extend statistical property predictions (e.g., masked language predictions) of word phrases by computing conditional probabilities pertaining to an association between a query phrase and a target phrase during an inference phase. In at least one embodiment, a conditional probability is computed as a softmax function that normalizes an output of one or more transformer-based language neural networks. In at least one embodiment, by using a transformer-based language neural network that is modified to compute conditional probabilities for query and target phrases, a transformer-based language neural network takes into account negation, flexible phrasing, and other context in which words are used, achieves high accuracy, and does not suffer from vocabulary and resulting computational problems. In at least one embodiment, one or more transformer-based language neural networks rank items of interest in a query phrase for a desirable property in a target phrase, reflecting latent domain-specific information in a large literature corpus.

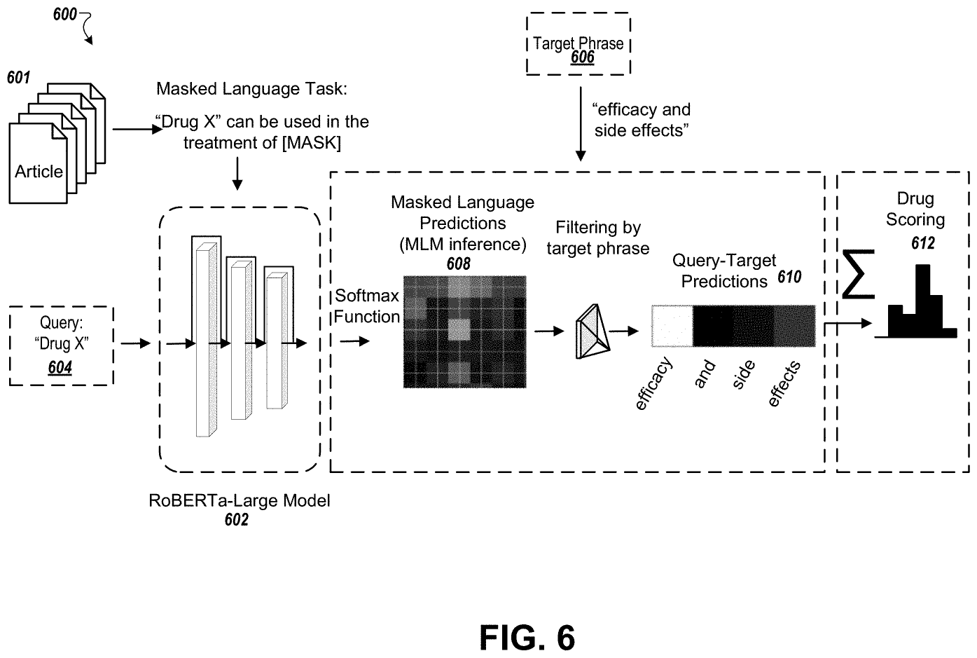

In at least one embodiment, as described in more detail below with respect to FIGS. 6-14, one or more exemplary transformer-based language neural networks are trained using domain-specific data such as well-curated datasets specific to pharmacology and, during an inference phase, are used to identify one or more medications or drugs described in one or more documents. In at least one embodiment, datasets specific to pharmacology can include a machine-readable, full-text literature dataset such as “CORD-19” that includes latent information that can be accessed for drug discovery concerning COVID-19. A quality of CORD-19 dataset is attributed to an availability of full-text access on literature with a narrow focus that was not available in previous medical fine-tuning studies. In at least one embodiment, a first portion of “CORD-19” dataset (e.g., 80%) is used to train one or more transformer-based language neural networks and a second portion (e.g., 20%) is used to further tune one or more transformer-based language neural networks.

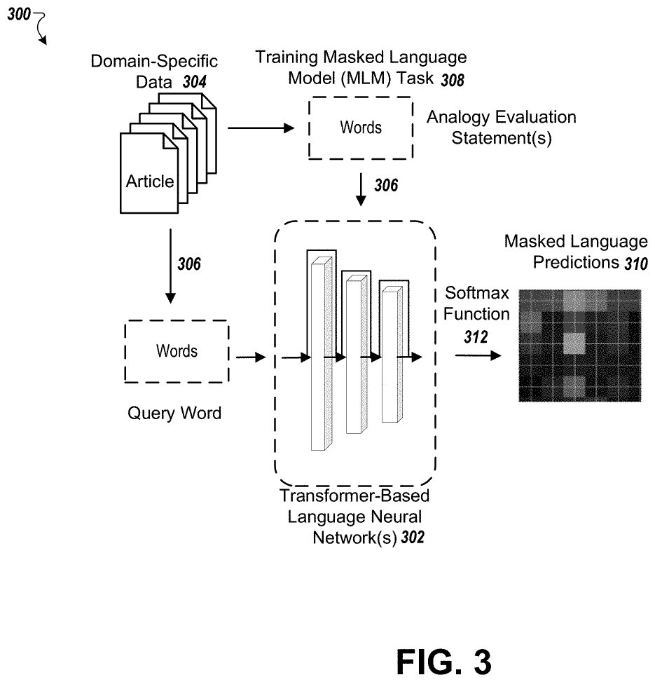

FIG. 3. is an example data flow diagram for a process 300 to train one or more transformer-based language neural networks 302 using domain-specific data 304, in accordance with at least one embodiment. In at least one embodiment, during a training phase, one or more transformer-based language neural networks 302 receive input words 306, such as a sequence of words, from domain-specific data 304 and encode input words 306 into vectors in a vector space. In at least one embodiment, a vector includes one or more tokens corresponding to one or more words of input words 306. In at least one embodiment, one or more transformer-based language neural networks 302 include one or more layers and an input layer performs tokenization of input words 306 into vectors in a vector space. In at least one embodiment, an input layer uses byte-pair encoding (BPE) to tokenize input words 306 into vectors. In at least one embodiment, BPE tokenization can perform subword tokenization in which one or more words can be split into subwords for tokenization. In at least one embodiment, a 50K byte-pair encoding tokenization is used for one or more transformer-based language neural networks 302. In at least one embodiment, one or more transformer-based language neural networks 302 include a default set of hyper-parameters. In at least one embodiment, one or more transformer-based language neural networks 302 include an input layer that creates vectors corresponding to input words, one or more hidden layers process these vectors according to context of words, and an output layer that computes probabilities of words with context, such as in a form of a softmax function. In at least one embodiment, a softmax function can include a normalizer. In at least one embodiment, as described below, a softmax function can compute conditional probabilities of a target phrase of one or more words given a query phrase of one or more words. In at least one embodiment, one or more transformer-based language neural networks 302 can use a softmax function in forward propagation. In at least one embodiment, one or more transformer-based language neural networks 302 can use other cross entropy functions. In at least one embodiment, one or more transformer-based language neural networks 302 are based on one or more RoBERTa neural networks that are trained on domain-specific data. In at least one embodiment, a RoBERTa neural network uses a language masking strategy that learns to predict intentionally hidden sections of text within unannotated language. In at least one embodiment, one or more transformer-based language neural networks 302 include ELECTRA transformers, BERT transformers, XLNet transformers, or similar transformers. In at least one embodiment, one or more transformer-based language neural networks 302 includes a transformer with multiple attention mechanisms, such as a first attention mechanism for one or more query words and a second attention mechanism for one or more target words. In at least one embodiment, one or more transformer-based language neural networks 302 are pre-trained on a large dataset using an unsupervised pre-training objective and then fine-tuned with domain-specific data for a specific task of identifying one or more relationships between one or more input words, such as one or more query words, and one or more target words for knowledge discovery in domain-specific data. In at least one embodiment, one or more transformer-based language neural networks 302 are fine-tuned with supervised training or semi-supervised training. In at least one embodiment, one or more transformer-based language neural networks 302 are fine-tuned with a k-shot method.

In at least one embodiment, one or more transformer-based language neural networks 302 include a query-target conditioning layer that uses a softmax function to compute a conditional probability for each target word in a target phrase given a query word in a query phrase, and a summation layer to sum a conditional probability for each target word in a target phrase to obtain a score indicating a quantified relationship between a query word and a target phrase.

In at least one embodiment, one or more transformer-based language neural networks 302 include a layer to: compute a first masked language prediction for a query word; compute a second masked language prediction for each target word in a target phrase; perform a dot product multiplication of a first masked language prediction and a second masked language prediction to filter a first masked language prediction and a second masked language prediction to obtain query-target predictions for a target phrase; and sum a query-target predictions for a target phrase to obtain a score indicating a quantified relationship between a query word and a target phrase. In at least one embodiment, one or more query-target predictions each includes a first query-target prediction that can be a positive number indicating a positive relationship between a query word and a corresponding target word in a target phrase. In at least one embodiment, one or more query-target predictions each include a first query-target prediction that is a negative number indicating a negative relationship between a query word and a corresponding target word in a target phrase.

In at least one embodiment, one or more transformer-based language neural networks 302 include a scoring function to sum and normalize a score of an association between each word of a query phrase and each word of a target phrase. In at least one embodiment, one or more transformer-based language neural networks 302 include a ranking function to rank an item of interest in a query phrase for a desirable property in a target phrase.

In at least one embodiment, one or more transformer-based language neural networks 302 receive a query phrase of one or more words and a target phrase of one or more words. In at least one embodiment, one or more transformer-based language neural networks include a scoring function to sum and normalize a score of an association between each word of a query phrase and each word of a target phrase. In at least one embodiment, one or more transformer-based language neural networks include a ranking function to rank an item of interest in a query phrase for a desirable property in a target phrase.

In at least one embodiment, one or more transformer-based language neural networks 302 are pre-trained models with a masked language modeling (MLM) objective. In at least one embodiment, a MLM objective takes a sentence and randomly masks a percentage of words in an input and predicts mask words, allowing one or more transformer-based language neural networks 302 to learn a bidirectional representation of a sentence. In at least one embodiment, a bidirectional representation can be used to extract features that are useful for downstream tasks. In at least one embodiment, one or more transformer-based language neural networks 302 are pre-trained with one or more analogy evaluations, including semantic analogies and domain-specific analogies, such as antiviral drug analogies described in more detail below.

In at least one embodiment, one or more transformer-based language neural networks 302 perform a dot product multiplication on a first output of a first attention mechanism and a second output of a second attention mechanism to obtain an output that includes one or more conditional probabilities of each of one or more target words for each of one or more query words. In at least one embodiment, one or more transformer-based language neural networks 302 are trained using a k-shot learning approach. In at least one embodiment, one or more transformer-based language neural networks 302 are fine-tuned for a classification task using only k examples as described in more detail below with k-shot RoBERTa-large models used for drug discovery in a large literature dataset.

In at least one embodiment, during a training phase, a masked language model (MLM) task 308 performs training by replacing a percentage of tokens, e.g., 13.5%, with a <mask> token or a corrupted token. In at least one embodiment, for tokens that are replaced, 90% of tokens are replaced with a <mask> token and 10% of tokens are replaced with a corrupted token. Alternatively, other percentage of masked tokens and corrupted tokens can be used. In at least one embodiment, during a training phase, domain-specific data 304 can be dynamically masked a number of times, such as ten times. In at least one embodiment, one or more transformer-based language neural networks 302 use a cross-entropy loss for MLM predictions 310. In at least one embodiment, an MLM prediction 310 is used for analogy evaluation with a text prompt, such as “A is to B as C is to <mask>” or other analogy evaluation statements. In at least one embodiment, one or more transformer-based language neural networks 302 are trained using domain-specific data 304 to identify one or more relationships among one or more input words 306. In at least one embodiment, one or more transformer-based language neural networks 302 are trained on “CORD-19” dataset and after a number of training steps (e.g., 100,000 steps), a MLM task reaches a perplexity (e.g., 2.4696) on a percentage of text (e.g., 20%) that is reserved for testing during language modelling in a training phase.

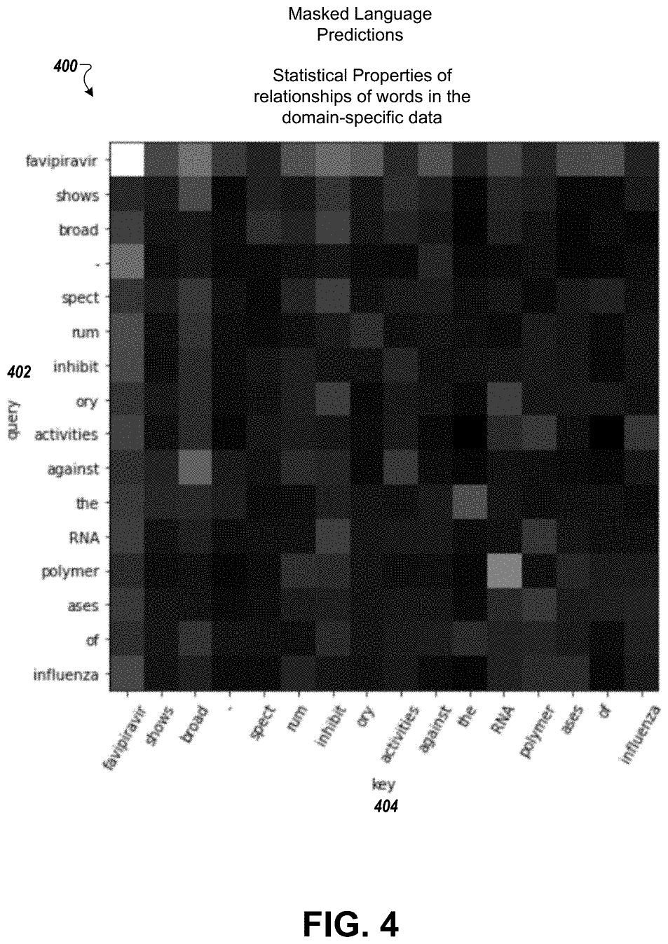

In at least one embodiment, relationships among input words 306 can include a relationship between a query phrase of one or more words and a target phrase of one or more words. In at least one embodiment, relationships among words can be used to discover latent domain-specific information in domain-specific data 304. In at least one embodiment, one or more transformer-based language neural networks 302 include one or more layers for vocabulary generation and evaluation for MLM predictions 310. In at least one embodiment, one or more transformer-based language neural networks 302 can compute MLM predictions 310 using a softmax function 312. In at least one embodiment, after a training phase, one or more transformer-based language neural networks 302 can compute MLM predictions 310. An example of MLM predictions 310 are illustrated and described below with respect to FIG. 4.

FIG. 4 is an example attention visualization 400 of statistical properties of relationships of words in domain-specific data for masked language predictions, in accordance with at least one embodiment. Attention visualization 400 includes a self-sequence to sequence, which includes a first input sequence corresponding to an input phrase of words and a second input sequence corresponding to a same input phrase of words, where a masked language prediction is computed for each token in a first input sequence given each token in a second input sequence. In this illustrated example, one or more transformer-based language neural networks 302 receive an input sentence from domain-specific data, such as “Importantly, favipiravir triphosphate shows broad-spectrum inhibitory activities against the RNA polymerases of influenza A viruses (including the highly pathogenic H5N1 viruses) (330, 333) and many other positive-sense RNA and negative-sense RNA viruses efficacy (331)” and examines a corresponding sequence-to-sequence attention by computing statistical properties of relationships of words in an input sentence against itself. As illustrated in attenuation visualization 400, each row includes a word or subword of a sequence as a query phrase 402 and each column includes a word or a subword of a sequence as a target phrase 404, and each word or subword is a token. Each cell of attention visualization 400 plots a per-token attention, where white is a positive association, black is a negative association, and different shades in between represent different levels of association. A per-token attention is based on MLM predictions as described above. In at least one embodiment, a per-token attention is computing a cross-entropy loss, as expressed in equation (1):

$$A t = \frac{exp ( {H}_{θ}^{t} ( {x}_{t} ) e ( {x}_{t} ) )}{{\sum }_{j} exp ( {H}_{θ}^{j} ( {x}_{t} ) e ( {x}_{t} ) )} \qquad ( 1 )$$

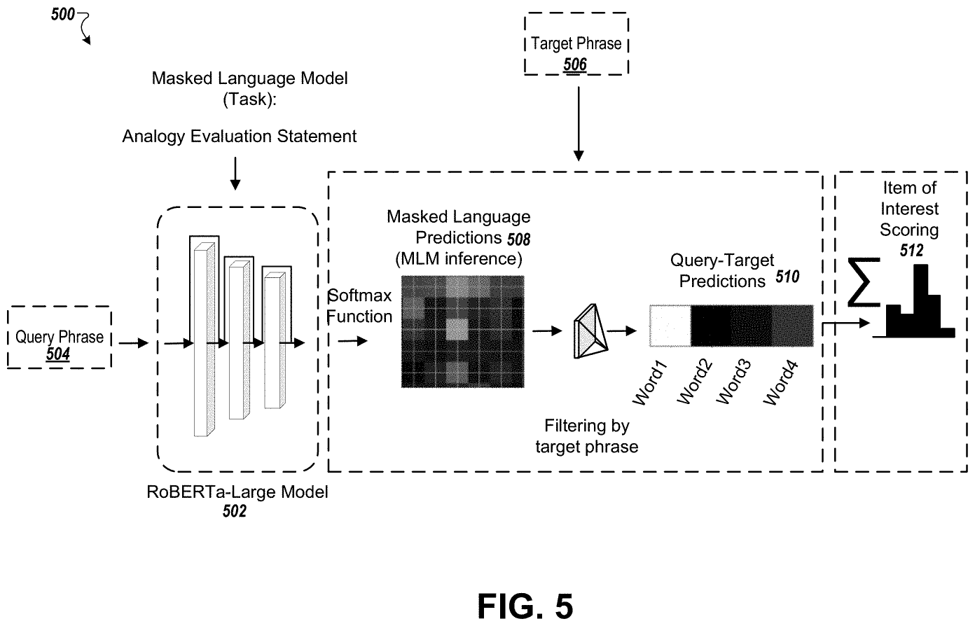

FIG. 5 is an example data flow diagram for a process 500 to use one or more transformer-based language neural networks 502 to identify one or more relationships among one or more words, in accordance with at least one embodiment. In at least one embodiment, during an inference phase, a query phrase 504 and a target phrase 506 are selected based on relationships of interest. In at least one embodiment, a relationship with a RoBERTa training objective is expressed as in equation (2):

$${max}_{θ} log {p}_{θ} ( \overline{x} \vert \hat{x} ) = {\sum }_{t} δlog \frac{exp ( {H}_{θ}^{t} ( \hat{{x}_{t}} ) e ( {x}_{t} ) )}{{\sum }_{{x}^{′}} exp ( {H}_{θ}^{j} ( {\hat{x}}_{t} ) e ( {x}_{t} ) )} \qquad ( 2 )$$

where δ indicates a masked token and 0 otherwise, x∈[x₁, . . . , x_{T}] is a text sequence, \hat{x} represents a corrupted token, \tilde{x} represents a masked token, e(x) is an embedding of a text sequence, and H_{θ} is a function of a RoBERTa-large architecture that maps a T length text sequence into a sequence of hidden vectors. In at least one embodiment, training objective results are optimized in an accurate MLM inference with masked language predictions 508 by one or more transformer-based language neural networks 502. In at least one embodiment, for an MLM inference, masked tokens q∈[q₁, . . . , q_{L}] are targeted for masked language predictions 508 (also referred to as token predictions), as expressed in equation (3):

$${P}_{K} = \frac{exp ( {H}_{θ}^{k} ( {q}_{k} ) )}{{\sum }_{j} exp ( {H}_{θ}^{j} ( {q}_{k} ) )} . \qquad ( 3 )$$

In at least one embodiment, an attention relationship for self-sequence to sequence attention is computed per equations (1) and (3) and illustrated in attention visualization 400 of FIG. 4.

In at least one embodiment, for a query-target (QT) prediction 510, masked token targets are conditioned on query targets y∈[y₁, . . . , y_{L}] as expressed in equation (4):

$$R := {P}_{K} ( {q}_{k} \vert {q}_{k} ∈ y ) ) = \frac{{\sum }_{l} exp ( {H}_{θ}^{l} ( {q}_{k} ) )}{{\sum }_{j} exp ( {H}_{θ}^{j} ( {q}_{k} ) )} . \qquad ( 4 )$$

In at least one embodiment, during an inference phase, a QT prediction 510 follows an independent assumption in equation (2) and therefore is contained in a training objective. That is, an accurate QT prediction is implied. In at least one embodiment, when q=y, a QT conditioning method decomposes to an MLM task prediction. In at least one embodiment, a QT conditioning method is more focused than reformulating a span prediction method due to rejection of extraneous tokens that would be admitted in a dot product formulation. In at least one embodiment, a QT prediction is a dot product multiplication of attention relationships of a query phrase and attention relationships of a target phrase. In at least one embodiment, once a QT prediction 510 has been formed, an analogy MLM task may be permuted with words from query phrase 504 and target phrase 506 using “Q is to T as Q is to <mask>” to analyze a top-k related terms without conditioning. In at least one embodiment, for rank prediction, tokens with positive and negative associations are not intentionally mixed as they are for visualization purposes. As described above, a transformer attention visualization examines a self-sequence to sequence attention, plotting a per-token attention, as expressed in equation (1). In at least one embodiment, for QT attenuation visualization, token attention per query-target phrases are expressed in equation (5):

$${R}_{l} = \frac{exp ( {H}_{θ}^{l} ( {q}_{k} ) ) e ( {x}_{t} )}{{\sum }_{j} exp ( {H}_{θ}^{j} ( {q}_{k} ) ) e ( {x}_{t} )} . \qquad ( 5 )$$

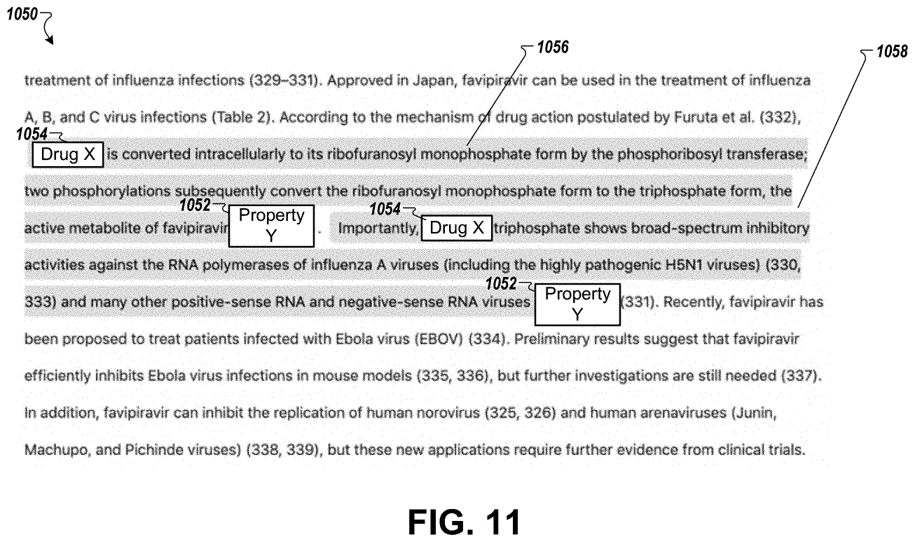

In at least one embodiment, QT scoring is adapted to sentence highlighting, such as illustrated and described below with respect to FIG. 11. In at least one embodiment, QT scoring includes negative associations, providing negation handling as an expected result of QT scoring, as compared to a span extraction or abstractive summarization method that does not determine negative associations. In at least one embodiment, a QT scoring method provides negative associations as an advancement over skip-gram word2vec scoring method. In at least one embodiment, a forward chaining (FC) analysis can be performed on inference data, such as time series data. In at least one embodiment, one or more transformer-based language neural networks 502 use a query-target condition method to isolate and extract relevant information from a dataset. In at least one embodiment, one or more transformer-based language neural networks 502 use a method that extends a masked language token prediction using query-target conditioning to treat a specificity challenge. In at least one embodiment, an inference by one or more transformer-based language neural networks 502 is modified to condition on softmax probabilities in a target phrase (e.g., word1, word2, word3, word4). In at least one embodiment, one or more transformer-based language neural networks 502 implement a scoring function 512 that include summation and normalization over query phrase 504 and target phrase 506. In at least one embodiment, items of interest in query phrase 504 for a desirable property in target phrase 506 are ranked based on a scoring function 512. In at least one embodiment, domain-specific knowledge can be mined using one or more transformer-based language neural networks 502 by performing prediction discovery on terms that are statistically present in a large corpus of data that are difficult to discover by a layperson or expert practitioners. In at least one embodiment, performing prediction discovery using one or more transformer-based language neural networks 502 can enable latent knowledge to be accessed, leading to faster discovery in materials, methods, and rankings of properties within a domain-specific data. In at least one embodiment, a rank calculation from equation (4) is performed on a year-limited data. In at least one embodiment, for example, a target query is set as “clinical trials efficacy” and candidate drugs are selected from a clinical trials dataset, such as described in more detail below with respect to FIGS. 6-14.

FIG. 6 is an example data flow diagram for a process 600 to use one or more transformer-based language neural networks 602, which are trained using domain-specific data 601, to identify one or more drugs described in one or more documents, in accordance with at least one embodiment. In at least one embodiment, one or more transformer-based language neural networks 602 identify and rank one or more drug candidates from a clinical trials dataset based, at least in part, on conditional probabilities for an association between each of drug candidates and at least one target property.

In at least one embodiment, during an inference phase, a query phrase 604 and a target phrase 606 are selected based on relationships of interest. In at least one embodiment, one or more transformer-based language neural networks 602 perform a masked language task, such as “Drug X can be used in treatment of [MASK],” with query phrase 604 being “Drug X” and target phrase 606 being “efficacy and side effects.” In at least one embodiment, as described above with respect to equation (2), a function of one or more transformer-based language neural networks 602 maps a T length text sequence into a sequence of hidden vectors. In at least one embodiment, optimizing a training objective results in an accurate MLM inference 608 as expressed above in equation (3). In at least one embodiment, for a query-target prediction 610, masked token targets are conditioned on query targets as expressed in equation (4). In at least one embodiment, masked language predictions 608 are filtered by target phrase 606, resulting in query-target predictions 610, including an association between each word of query phrase 604 and each word of target phrase 606. As illustrated in FIG. 6, “efficacy” from target phrase 606 has a positive association with “Drug X” from query phrase 604, and “and” and “side” from target phrase 606 have negative associations with “Drug X” from query phrase 604. “Effects” from target phrase 606 has an association that is in between associations of “efficacy” and “side” from target phrase 606. In at least one embodiment, for a rank prediction, one or more transformer-based language neural networks 602 score “Drug X” based on associations computed in query-target predictions 610. In at least one embodiment, one or more transformer-based language neural networks 602 sum and normalize query-target predictions 610 in a drug scoring function 612 for multiple drug candidates as query phrase 604 with target phrase 606 being set to “efficacy and side effects.”

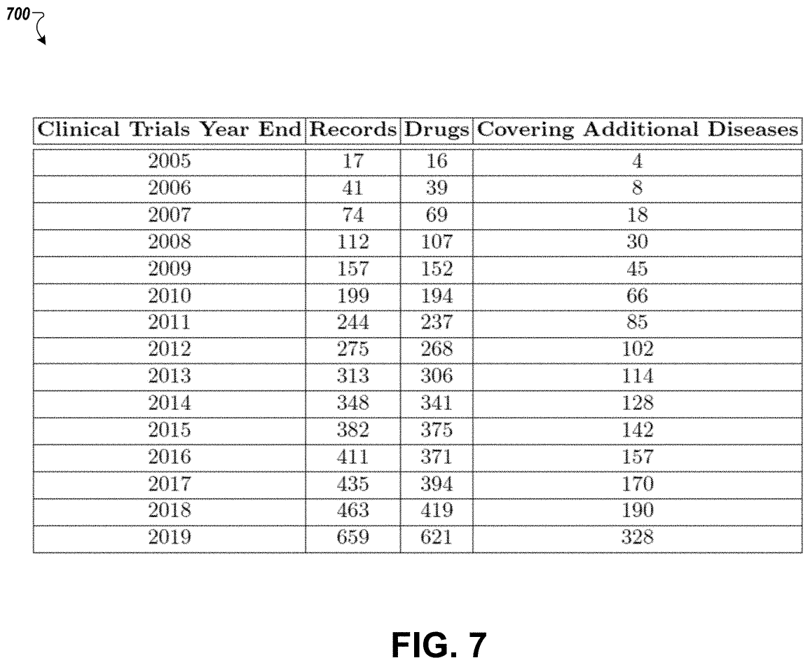

In at least one embodiment, during a training phase, a MLM task performs training by replacing a percentage of tokens, e.g., 13.5%, with a <mask> token or a corrupted token. In at least one embodiment, for tokens that are replaced, 90% of tokens are replaced with a <mask> token and 10% of tokens are replaced with a corrupted token. In at least one embodiment, a cross-entropy loss is used for prediction by one or more transformer-based language neural networks 602. In at least one embodiment, one or more transformer-based language neural networks 602 use a 50K byte-pair encoding tokenization. In at least one embodiment, domain-specific data 601 includes “CORD-19” dataset and one or more transformer-based language neural networks 602 use clinical trials dataset for inference data. In at least one embodiment, inputs from CORD-19 dataset are dynamically masked ten times. Clinical trials dataset can include United States Food and Drug Administration (FDA) approved drugs and global clinical trials data, such as summarized in FIG. 7.

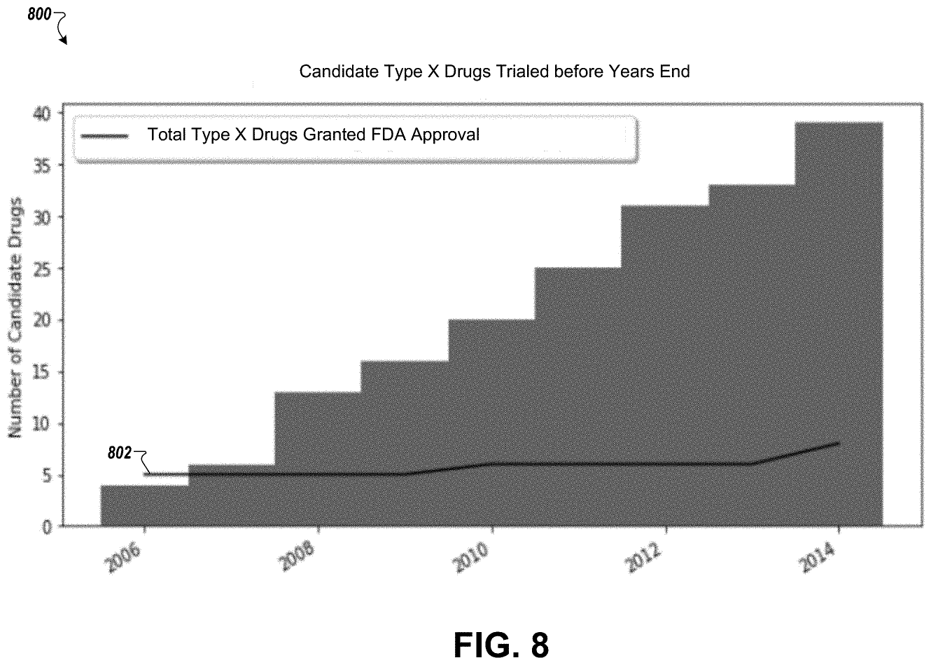

FIG. 7 is a table 700 showing a summary of a sample clinical trials dataset, in accordance with at least one embodiment. Table 700 includes counts for a number of trials and drugs trialed per year's end. In at least one embodiment, using influenza as a condition, one or more transformer-based language neural networks 602 can be used to check a search term to filter by drug treatments and focus on years prior to 2016, when a last antiviral drug was approved. In at least one embodiment, de-duplication is performed on trade and scientific names using a chart from clinical trials dataset. A number of drug candidates and approved drugs specifically for influenza per year are plotted in FIG. 8. In at least one embodiment, other specific types of drugs can be used.

FIG. 8 is an example graph 800 illustrating a number of candidate drugs of a specific type trialed each year and a total of specific-type drugs (“Type X”) granted FDA approval over time, in accordance with at least one embodiment. In graph 800, line 802 represents specific-type drugs receiving FDA approval. For example, there are only eight antiviral drugs approved for influenza strains globally with a ninth drug, remdesivir, receiving emergency approval.

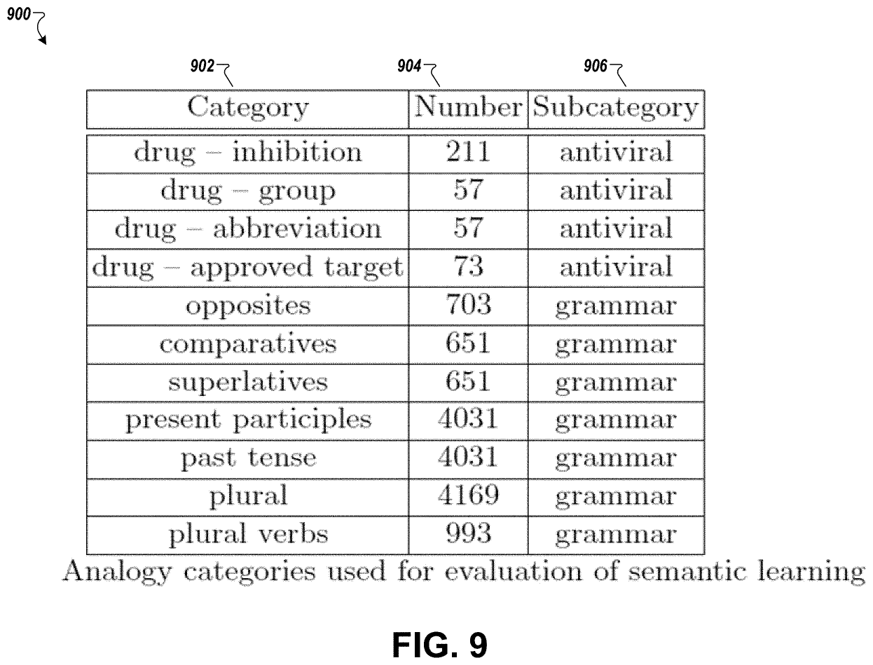

Referring back to FIG. 6, one or more transformer-based language neural networks 602 can perform analogy evaluations, including language analogies (e.g., grammar analogies) and drug analogies (e.g., antiviral analogies), such as illustrated and described below with respect to FIG. 9.

FIG. 9 is a sample table 900 of analogy categories used for evaluation of semantic learning, in accordance with at least one embodiment. In at least one embodiment, for analogy evaluation, a set of language analogies and drug analogies are drawn from relationships in clinical trials dataset. In at least one embodiment, as illustrated in table 900, each category 902 has a number of analogy evaluations 904 that can be performed, and a subcategory 906, such as antiviral or grammar analogy evaluations. In at least one embodiment, antiviral subcategory 906 includes a following list of categories 902: “drug—inhibition,” “drug—group,” “drug—abbreviation,” and “drug—approved target.” In at least one embodiment, grammar subcategory 906 includes a following list of categories 902: “opposites,” “comparatives,” “superlatives,” “present participles,” “past tense,” “plural,” and “plural verbs.” In at least one embodiment, for k-shot training, a random set of k=5 analogies from each category is used as additional pre-training for a MLM task.

Referring back to FIG. 6, during an inference phase, one or more transformer-based language neural networks 602 can perform an FC analysis on clinical trials dataset. In at least one embodiment, to preserve a nature of time-series data, a rank calculation from equation (5) is performed on year-limited data in graph 800, which includes a number of candidate influenza drugs trialed each year and a total of influenza drugs granted FDA approval over time. Query phrase 604 is set to each drug candidate from clinical trials dataset, such as from a number specified in column 2 of Table 700, and target phrase 606 is set to “clinical trials efficacy.”

In at least one embodiment, one or more transformer-based language neural networks 602 use a query-target condition method to isolate and extract relevant information from clinical trials dataset. In at least one embodiment, an inference by one or more transformer-based language neural networks 602 is modified to condition on softmax probabilities in a target phrase 606 (e.g., “clinical,” “trials,” and “efficacy”). In at least one embodiment, one or more transformer-based language neural networks 602 implement a scoring function 612 that include summation and normalization over drug candidates for each query phrase 604 and “clinical trials efficacy” for target phrase 606. In at least one embodiment, an item of interest, such as “efficacy,” for each drug candidate in query phrase 604 is ranked based on a scoring function 612.

In at least one embodiment, one or more transformer-based language neural networks 602 identify one or more drugs having at least one target property from a clinical trials dataset. In at least one embodiment, one or more transformer-based language neural networks determine a drug candidate for drug approval by ranking drug candidates from a clinical trials dataset based, at least in part, on query-target conditioning predictions of drug candidates as query words in a clinical trials dataset and an efficacy property as a target property in a clinical trials dataset.

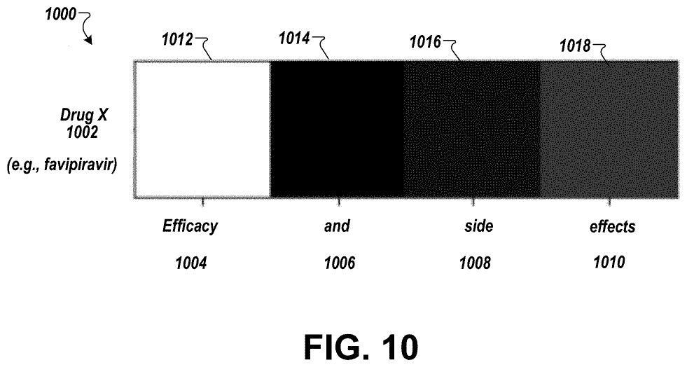

In at least one embodiment, during an inference phrase, one or more transformer-based language neural networks 602, which are trained on domain-specific data, can be modified during an inference phase to compute conditional probabilities for an association between a query phrase (e.g., Drug X) and a target phrase (e.g., “efficacy and side effects” and “clinical trials efficacy”). In at least one embodiment, one or more transformer-based language neural networks 602 compute conditional probabilities of associations between query phrase 604 and target phrase 605 using a softmax function. In at least one embodiment, using conditional probabilities, drug candidates are ranked to identify a subset of drug candidates that are likely to get approval by a reviewing authority, such FDA. In at least one embodiment, one or more transformer-based language neural networks 602 perform high-level sentiment analysis to determine a score and an MLM inference mines relationships between query phrase 604 and target phrase 606. In at least one embodiment, one or more transformer-based language neural networks 602 perform high-level sentiment analysis, using attention visualization methods, to determine a per-sentence passage highlighting score as illustrated and described in FIGS. 10-11.

FIG. 10 is an example attention visualization 1000 of statistical properties of relationships of a query word 1002 in domain-specific data and target words 1004-1008 of a target phrase, in accordance with at least one embodiment. In at least one embodiment, one or more transformer-based language neural networks can determine statistical properties of relationships of a query word 1002, “Drug X,” such as “favipiravir,” and target words 1004-1008, such as “efficacy,” “and,” “side,” “effects,” as illustrated in attention visualization 1000. In at least one embodiment, one or more transformer-based language neural networks can determine a relationship using a QT prediction representing a token attention per query-target for attention visualization 1000 based on equation (5). In at least one embodiment, attention visualization 1000 can be determined using a query-target function that shows positive (light) associations, such as association 1012 between “Drug X” as query word 1002 and “efficacy” as target word 1004, and negative (dark) associations, such as association 1014 between “Drug X” as query word 1002 and “and” as target word 1006. In at least one embodiment, attention visualization 1000 can also show associations that are between positive (light) associations and negative (dark) associations, such as association 1016 between “Drug X” as query word 1002 and “side” as target word 1008 and association 1018 between “Drug X” as query word 1002 and “effects” as target word 1010. In at least one embodiment, association 1016 has a more positive association with query word 1002 than association 1014, association 1018 has a more positive association with query word 1002 than association 1016, and association 1012 has a more positive association with query word 1002 than association 1018. Alternatively, other words can be used for query word 1002 and one or more other words can be used for a target phrase, and corresponding associations can be determined from query-target (QT) predictions with those input words.

FIG. 11 is an example passage 1050 in domain-specific data that is highlighted on a per-sentence basis using a target term 1052, in accordance with at least one embodiment. In at least one embodiment, one or more transformer-based language neural networks can determine QT scoring of target words for a given query word 1054, such as “Drug X.” In at least one embodiment, target term 1052, such as “Property Y” (e.g., efficacy, effects, inhibitor, or other target properties) is selected as an item of interest in a target phrase for a given query word 1054, and one or more transformer-based language neural networks can identify one or more sentences in example passage 1050 in domain-specific data based on QT scoring of target term 1052 for a given query word 1054. In at least one embodiment, target term 1052 can be more than one word, such as “side effects,” or “clinical trials efficacy.” In at least one embodiment, as illustrated in FIG. 11, one or more transformer-based language neural networks identify a first sentence 1056 and a second sentence 1058 in example passage 1050, both including target term 1052 (e.g., Property Y) and query word 1054 (e.g., Drug X). In at least one embodiment, second sentence 1058 corresponds to a sentence used in attenuation visualization 400 of FIG. 4, which includes a self-sequence to sequence visualization of associations between query words and target words (also referred to as keys or key words). In at least one embodiment, first sentence 1056, second sentence 1058, or any combination thereof can be identified using QT predictions for a target phrase given a query word, such as illustrated and described above with respect to attention visualization 1000 of FIG. 10, where a query word is “Drug X” (e.g., “favipiravir”) and a target phrase is “efficacy and side effects.”

In at least one embodiment, QT predictions or QT scoring can be used for analogy evaluations, including drug analogies (antiviral analogies) and semantic analogies (e.g., grammar analogies).

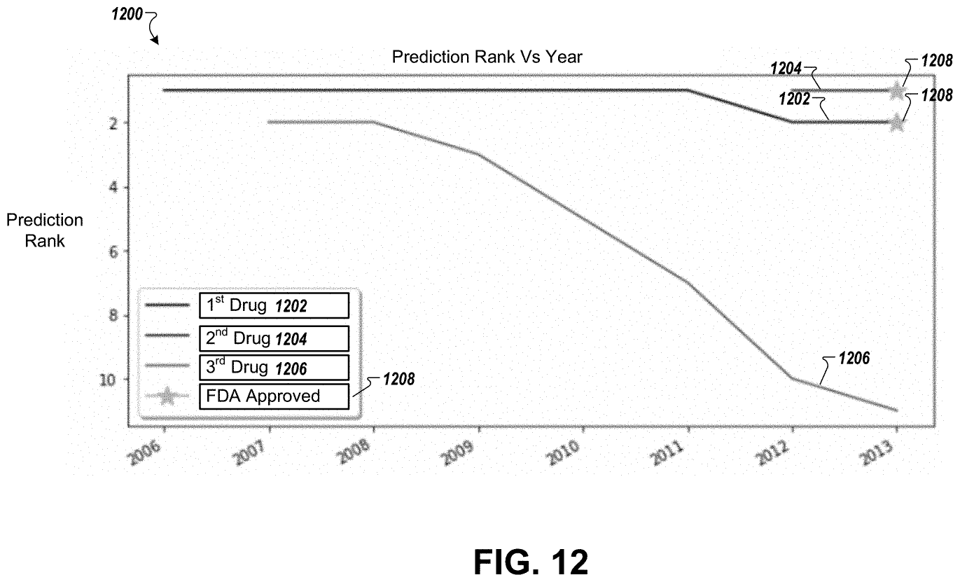

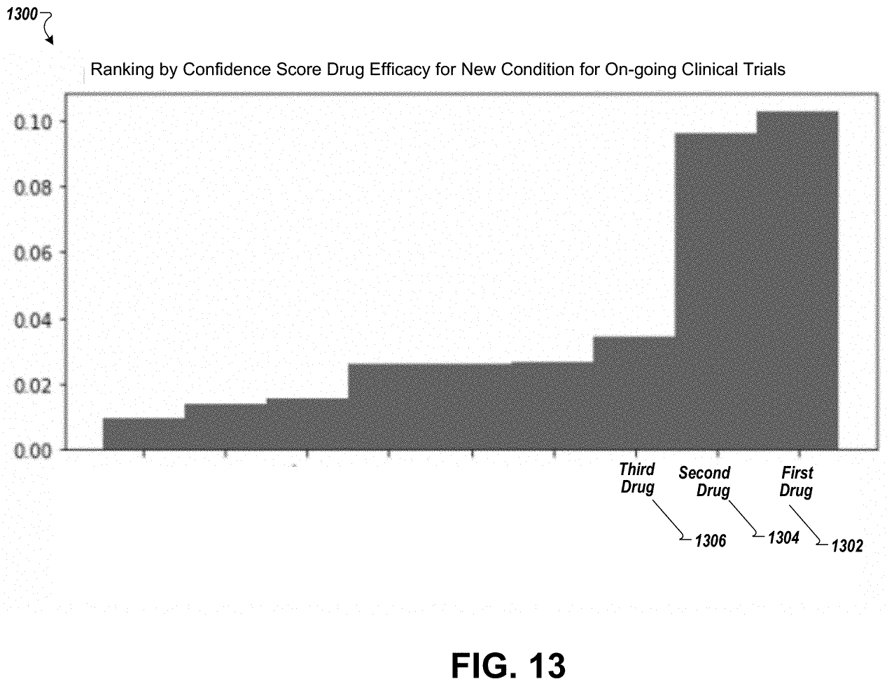

In at least one embodiment, CORD-19 RoBERTa-large model can capture synthetic analogies, such as antiviral analogies, and can be used for forward predictions in a forward chaining (FC) analysis, such as described below with respect to FIG. 12. In at least one embodiment, grammar analogies and antiviral analogies can be used for ranking drugs, such as described below with respect to FIG. 13.

FIG. 12 is an example graph 1200 illustrating a prediction rank of drug candidates over years and indications of FDA approval, in accordance with at least one embodiment. In at least one embodiment, one or more transformer-based language neural networks can determine a prediction rank of drug candidates over years and indications of FDA approval using a FC analysis. In at least one embodiment, graph 1200 shows FC analysis for a period where clinical trials data is reliably available (e.g., since 2005). In at least one embodiment, graph 1200 is a year-limited FC ranking analysis of drugs of a specific type under clinical trials for FDA approval, including a first drug 1202, a second drug 1204, and a third drug 1206. As illustrated, only two drugs received FDA approval 1208 in a period between 2005 and 2016, namely first drug 1202 and second drug 1204. Third drug 1206 did not receive FDA approval 1208.

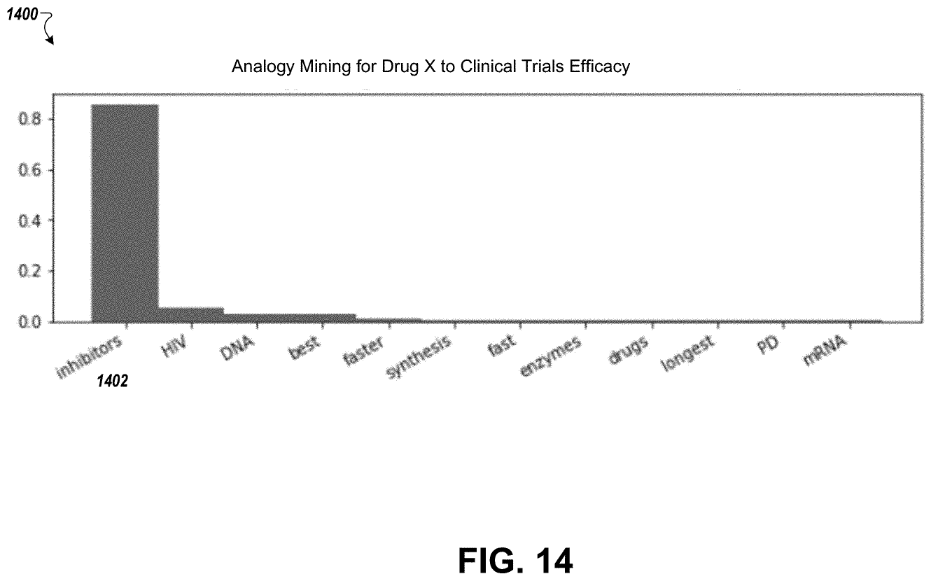

FIG. 13 is an example graph 1300 illustrating a ranking by confidence score of efficacy for a new condition for on-going clinical trials, in accordance with at least one embodiment. In at least one embodiment, one or more transformer-based language neural networks can determine a confidence score for each drug candidate for efficacy for on-going clinical trials. In at least one embodiment, graph 1300 shows confidence scores for a first drug 1302 (e.g., “remdesivir”), a second drug 1304 (e.g., “CD24FC”), a third drug 1306 (e.g., “hydroxychloroquine”) and other drugs. In at least one embodiment, graph 1300 shows first drug 1302 as having a higher confidence score than second drug 1304, third drug 1306, and other drugs. As a possible failure mode, third drug 1306 was ranked as a distant third as illustrated in FIG. 13 and may have been shown to have no correlation with positive or negative outcomes. In at least one embodiment, graph 1300 shows a ranking of current clinical trials for drug candidates for a new condition (e.g., COVID-19). In at least one embodiment, using a drug candidate as a query word, analogy mining can be done using target phrases, such as selected target phrases or sentences from passages in domain-specific data, such as illustrated in FIG. 14.

FIG. 14 is an example graph 1400 illustrating analogy mining for a drug to clinical trials efficacy, in accordance with at least one embodiment. In at least one embodiment, a permuted MLM task mines relationships that mirror a relationship of a drug (e.g., “Drug X”) as a query word and “clinical trials efficacy” as a target phrase. It should be noted that inverting an analogy mining operation (not illustrated in FIG. 14) does not recover a QT function as predicted terms are too generic to focus on drug candidate. In at least one embodiment, a method to test and verify results of negative associations of words in a target phrase, such as “side” and “effects” and/or drug combinations can be used, although not illustrated in FIG. 14. In at least one embodiment, one or more transformer-based language neural networks can be used for analogy mining for a particular drug, such as “remdesivir,” to clinical trials efficacy. In at least one embodiment, graph 1400 shows a ranking of QT predictions for target words like “inhibitors,” “HIV,” “DNA,” “best,” “faster,” “synthesis,” “fast,” “enzymes,” “drugs,” “longest,” “PD,” and “mRNA.” Target word 1402, “inhibitors,” has a higher QT prediction for “remdesivir” than other QT predictions in FIG. 14. In at least one embodiment, one or more transformer-based language neural networks can use a permuted MLM task to probe relationships of “remdesivir” with target words association with clinical trials efficacy.

In at least one embodiment, using one or more transformer-based language neural networks with a QT method brings specificity to discovery methods on a narrow literature dataset to predict clinical trials approval as verified by FC, real-time prediction, and relationship mining. In at least one embodiment, a QT method in one or more transformer-based language neural networks resembles a high-level sentiment analysis. In at least one embodiment, a QT method can be used in connection with visualization methods to determine a per-sentence passage highlighting score, such as illustrated and described above with respect to FIG. 11. In at least one embodiment, an MLM inference by one or more transformer-based language neural networks can mine relationships via k-shot tuning as illustrated in FIG. 12. In at least one embodiment, to refine relationship mining, a beam search decoder for multiple tokens can be used instead of single tokens. In at least one embodiment, a beam search decoder can increase expressiveness. In at least one embodiment, a scope of a QT method can be given since q, y∈X is a set of all statements in a domain-specific dataset and only finite sets could be generated. In at least one embodiment, for more validation, a gold standard for knowledge differentials at limits of research can be established. In at least one embodiment, a field of online learning may offer independent verification through datum valuation.

In at least one embodiment, besides accessible resource of clinical drug trials, other quantitative methods of determining drug function can be used for a given detailed dataset formulation. In at least one embodiment, methods could focus on canonical measures such as an inhibitory constant (K), effective dose at 95% (ED95), or number needed to treat (NNT). In at least one embodiment, protein receptor binding could require specialized dataset expertise for machine learning methods with appropriate domain-specific literature dataset for protein receptor binding. In at least one embodiment, one or more transformer-based language neural networks are used as a flexible tool in mining domain-specific literature.

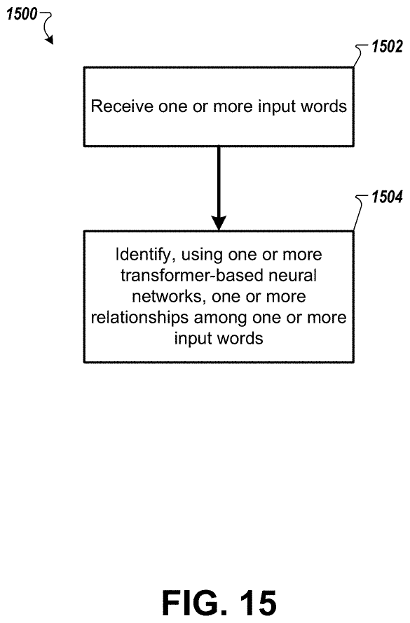

FIG. 15 is a flow diagram of a process 1500 to identify one or more relationships among one or more words using one or more transformer-based language neural networks trained using domain-specific data, in accordance with at least one embodiment. In at least one embodiment, process 1500 begins by receiving one or more input words, such as one or more query words and one or more target words for knowledge discovery in domain-specific data (block 1502). In at least one embodiment, process 1500 uses one or more transformer-based language neural networks to identify one or more relationships among one or more input words (block 1504). In at least one embodiment, one or more transformer-based language neural networks used to identify relationships between input words are trained using domain-specific data, such as a curated literature dataset. In at least one embodiment, one or more transformer-based language neural networks are trained using RoBERTa with domain-specific data. In at least one embodiment, a trained RoBERTa neural network computes query-target (QT) predictions for identifying relationships of one or more words, such as one or more query words and one or more target words, as described herein.

In at least one embodiment, process 1500, to identify relationships, computes a score indicating a quantified relationship between a query phrase of one or more words and a target phrase of one or more words. In at least one embodiment, a score is a positive number when a relationship is a positive relationship or positive association between a query phrase and a target phrase. In at least one embodiment, a score is a negative number when a relationship is a negative relationship or negative association between a query phrase and a target phrase.

In at least one embodiment, a score is a positive number indicating a positive relationship between a query word and a corresponding target word in a target phrase. In at least one embodiment, a score is a negative number indicating a negative relationship between a query word and a corresponding target word in a target phrase.

In at least one embodiment, one or more scores of items of interest can be used to discover latent domain-specific information in domain-specific data. In at least one embodiment, a quantified relationship can be represented as a percentage, a number, or other indications. In at least one embodiment, one or more transformed-based language neural networks include an input layer to receive additional domain-specific data during an inference phase, receive a query phrase of one or more words and encode a query phrase into a first vector of tokens using BPE, and receive a target phrase of one or more words and encode a target phrase into a second vector of tokens using BPE. In at least one embodiment, one or more transformed-based language neural networks include a BERT layer that is trained using a RoBERTa. In at least one embodiment, one or more transformed-based language neural networks include a first attention head to receive a first vector of tokens and compute a statistical prediction for each token in a first vector of tokens and a second attention head to receive a second vector of tokens and compute a statistical prediction for each token in a second vector of tokens. In at least one embodiment, one or more transformed-based language neural networks include an output layer to determine a query-target score by performing a dot product multiplication on statistical predictions of a first vector of tokens and statistical prediction of a second vector of tokens.

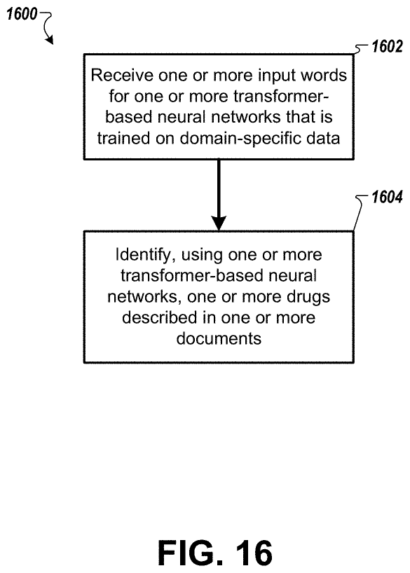

In at least one embodiment, one or more transformer-based language neural networks, which are trained using domain-specific data like CORD-19, identify one or more drugs described in one or more documents. In at least one embodiment, one or more transformer-based language neural networks can be trained on other domain-specific data and can identify an item of interest described in one or more documents as described herein. In at least one embodiment, one or more transformer-based language neural networks can be used to identify one or more drugs, such as for FDA approval such as described herein and with respect to FIG. 16.

FIG. 16 is a flow diagram of a process 1600 to identify one or more drugs described in one or more documents using a transformer-based language neural network that is trained using domain-specific data, in accordance with at least one embodiment. In at least one embodiment, process 1600 receives one or more input words for one or more transformer-based language neural networks that are trained on domain-specific data (e.g., pharmacology-specific data) (block 1602). In at least one embodiment, process 1600 identifies, using one or more transformer-based language network networks, one or more drugs in one or more documents (block 1604).

In at least one embodiment, process 1600, to identify one or more drugs, ranks one or more drug candidates from a clinical trials dataset based, at least in part, on conditional probabilities for an association between each of one or more drug candidates and at least one target property, such as “efficacy.” In at least one embodiment, process 1600, to identify one or more drugs, computes a score indicating a quantified relationship between a query word with a drug candidate and a target phrase of one or more words. In at least one embodiment, a score is a positive number when a relationship is a positive relationship or positive association between a drug candidate and a target phrase. In at least one embodiment, a score is a negative number when a relationship is a negative relationship or negative association between a drug candidate and a target phrase. In at least one embodiment, one or more transformed-based language neural networks include an input layer to receive a clinical trials dataset for a set of drugs during an inference phase. In at least one embodiment, for each drug of a set of drugs, an input layer receives a query word corresponding to a respective drug and encodes a query word into a first vector using BPE and receives a target phrase of one or more words and encodes a target phrase into a second vector of tokens using BPE. In at least one embodiment, one or more transformed-based language neural networks include a BERT layer that is trained using a RoBERTa and is modified during an inference phase to determine a drug score for each drug of a set of drugs. In at least one embodiment, one or more transformed-based language neural networks include an output layer to rank a set of drugs according to drug scores.

In at least one embodiment, one or more transformer-based language neural networks are trained using domain-specific data, such as CORD-19. In at least one embodiment, one or more transformer-based language neural networks are trained using RoBERTa with domain-specific data. In at least one embodiment, a trained RoBERTa neural network computes query-target (QT) predictions for drug scores and ranks drug candidates according to drug scores for identifying drugs in one or more documents, as described herein.

### Data Center

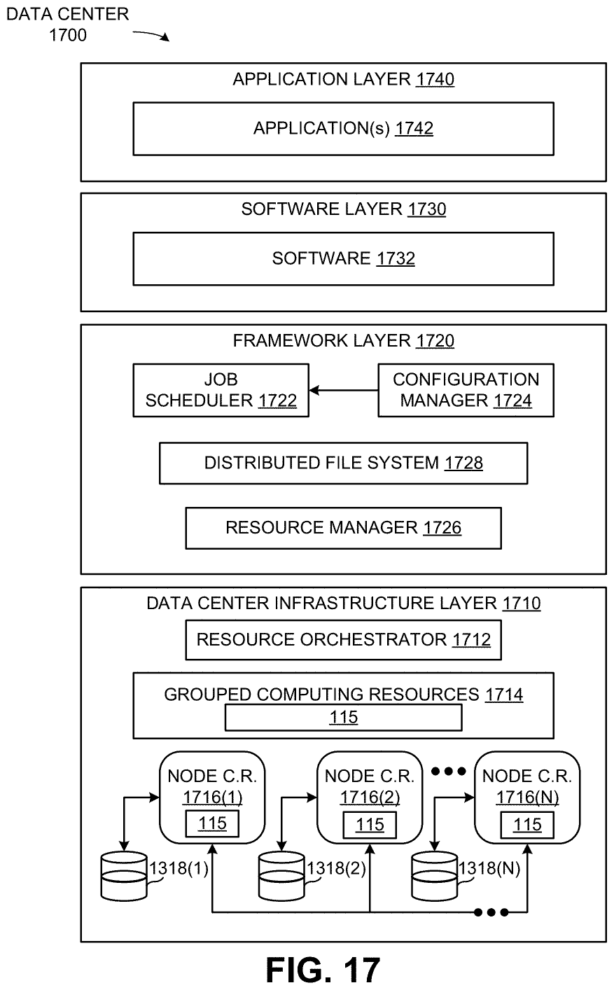

FIG. 17 illustrates an example data center 1700, in which at least one embodiment may be used. In at least one embodiment, data center 1700 includes a data center infrastructure layer 1710, a framework layer 1720, a software layer 1730, and an application layer 1740.

In at least one embodiment, as shown in FIG. 17, data center infrastructure layer 1710 may include a resource orchestrator 1712, grouped computing resources 1714, and node computing resources (“node C.R.s”) 1716(1)-1716(N), where “N” represents a positive integer (which may be a different integer “N” than used in other figures). In at least one embodiment, node C.R.s 1716(1)-1716(N) may include, but are not limited to, any number of central processing units (“CPUs”) or other processors (including accelerators, field programmable gate arrays (FPGAs), graphics processors, etc.), memory storage devices 1718(1)-1718(N) (e.g., dynamic read-only memory, solid state storage or disk drives), network input/output (“NW I/O”) devices, network switches, virtual machines (“VMs”), power modules, and cooling modules, etc. In at least one embodiment, one or more node C.R.s from among node C.R.s 1716(1)-1716(N) may be a server having one or more of above-mentioned computing resources.

In at least one embodiment, grouped computing resources 1714 may include separate groupings of node C.R.s housed within one or more racks (not shown), or many racks housed in data centers at various geographical locations (also not shown). In at least one embodiment, separate groupings of node C.R.s within grouped computing resources 1714 may include grouped compute, network, memory or storage resources that may be configured or allocated to support one or more workloads. In at least one embodiment, several node C.R.s including CPUs or processors may grouped within one or more racks to provide compute resources to support one or more workloads. In at least one embodiment, one or more racks may also include any number of power modules, cooling modules, and network switches, in any combination.

In at least one embodiment, resource orchestrator 1712 may configure or otherwise control one or more node C.R.s 1716(1)-1716(N) and/or grouped computing resources 1714. In at least one embodiment, resource orchestrator 1712 may include a software design infrastructure (“SDI”) management entity for data center 1700. In at least one embodiment, resource orchestrator 1712 may include hardware, software or some combination thereof.

In at least one embodiment, as shown in FIG. 17, framework layer 1720 includes a job scheduler 1722, a configuration manager 1724, a resource manager 1726 and a distributed file system 1728. In at least one embodiment, framework layer 1720 may include a framework to support software 1732 of software layer 1730 and/or one or more application(s) 1742 of application layer 1740. In at least one embodiment, software 1732 or application(s) 1742 may respectively include web-based service software or applications, such as those provided by Amazon Web Services, Google Cloud and Microsoft Azure. In at least one embodiment, framework layer 1720 may be, but is not limited to, a type of free and open-source software web application framework such as Apache Spark™ (hereinafter “Spark”) that may utilize distributed file system 1728 for large-scale data processing (e.g., “big data”). In at least one embodiment, job scheduler 1722 may include a Spark driver to facilitate scheduling of workloads supported by various layers of data center 1700. In at least one embodiment, configuration manager 1724 may be capable of configuring different layers such as software layer 1730 and framework layer 1720 including Spark and distributed file system 1728 for supporting large-scale data processing. In at least one embodiment, resource manager 1726 may be capable of managing clustered or grouped computing resources mapped to or allocated for support of distributed file system 1728 and job scheduler 1722. In at least one embodiment, clustered or grouped computing resources may include grouped computing resources 1714 at data center infrastructure layer 1710. In at least one embodiment, resource manager 1726 may coordinate with resource orchestrator 1712 to manage these mapped or allocated computing resources.

In at least one embodiment, software 1732 included in software layer 1730 may include software used by at least portions of node C.R.s 1716(1)-1716(N), grouped computing resources 1714, and/or distributed file system 1728 of framework layer 1720. In at least one embodiment, one or more types of software may include, but are not limited to, Internet web page search software, e-mail virus scan software, database software, and streaming video content software.

In at least one embodiment, application(s) 1742 included in application layer 1740 may include one or more types of applications used by at least portions of node C.R.s 1716(1)-1716(N), grouped computing resources 1714, and/or distributed file system 1728 of framework layer 1720. In at least one embodiment, one or more types of applications may include, but are not limited to, any number of a genomics application, a cognitive compute, application and a machine learning application, including training or inferencing software, machine learning framework software (e.g., PyTorch, TensorFlow, Caffe, etc.) or other machine learning applications used in conjunction with one or more embodiments.

In at least one embodiment, any of configuration manager 1724, resource manager 1726, and resource orchestrator 1712 may implement any number and type of self-modifying actions based on any amount and type of data acquired in any technically feasible fashion. In at least one embodiment, self-modifying actions may relieve a data center operator of data center 1700 from making possibly bad configuration decisions and possibly avoiding underutilized and/or poor performing portions of a data center.

In at least one embodiment, data center 1700 may include tools, services, software or other resources to train one or more machine learning models or predict or infer information using one or more machine learning models according to one or more embodiments described herein. For example, in at least one embodiment, a machine learning model may be trained by calculating weight parameters according to a neural network architecture using software and computing resources described above with respect to data center 1700. In at least one embodiment, trained machine learning models corresponding to one or more neural networks may be used to infer or predict information using resources described above with respect to data center 1700 by using weight parameters calculated through one or more training techniques described herein.

In at least one embodiment, data center may use CPUs, application-specific integrated circuits (ASICs), GPUs, FPGAs, or other hardware to perform training and/or inferencing using above-described resources. Moreover, one or more software and/or hardware resources described above may be configured as a service to allow users to train or performing inferencing of information, such as image recognition, speech recognition, or other artificial intelligence services.

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, inference and/or training logic 115 may be used in system FIG. 17 for inferencing or predicting operations based, at least in part, on weight parameters calculated using neural network training operations, neural network functions and/or architectures, or neural network use cases described herein.

### Computer Systems

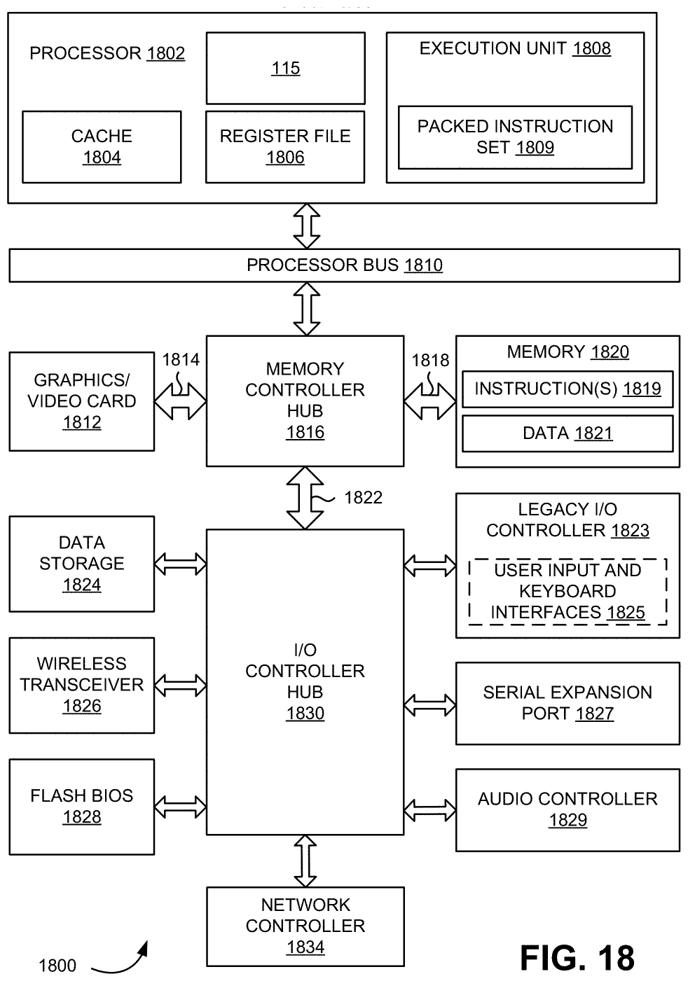

FIG. 18 is a block diagram illustrating an exemplary computer system, which may be a system with interconnected devices and components, a system-on-a-chip (SOC) or some combination thereof formed with a processor that may include execution units to execute an instruction, according to at least one embodiment. In at least one embodiment, a computer system 1800 may include, without limitation, a component, such as a processor 1802 to employ execution units including logic to perform algorithms for process data, in accordance with present disclosure, such as in embodiment described herein. In at least one embodiment, computer system 1800 may include processors, such as PENTIUM® Processor family, Xeon™ Itanium®, XScale™ and/or StrongARM™, Intel® Core™, or Intel® Nervana™ microprocessors available from Intel Corporation of Santa Clara, Calif., although other systems (including PCs having other microprocessors, engineering workstations, set-top boxes and like) may also be used. In at least one embodiment, computer system 1800 may execute a version of WINDOWS operating system available from Microsoft Corporation of Redmond, Wash., although other operating systems (UNIX and Linux, for example), embedded software, and/or graphical user interfaces, may also be used.

Embodiments may be used in other devices such as handheld devices and embedded applications. Some examples of handheld devices include cellular phones, Internet Protocol devices, digital cameras, personal digital assistants (“PDAs”), and handheld PCs. In at least one embodiment, embedded applications may include a microcontroller, a DSP, system on a chip, network computers (“NetPCs”), set-top boxes, network hubs, wide area network (“WAN”) switches, or any other system that may perform one or more instructions in accordance with at least one embodiment.

In at least one embodiment, computer system 1800 may include, without limitation, processor 1802 that may include, without limitation, one or more execution units 1808 to perform machine learning model training and/or inferencing according to techniques described herein. In at least one embodiment, computer system 1800 is a single processor desktop or server system, but in at least one other embodiment, computer system 1800 may be a multiprocessor system. In at least one embodiment, processor 1802 may include, without limitation, a complex instruction set computer (“CISC”) microprocessor, a reduced instruction set computing (“RISC”) microprocessor, a very long instruction word (“VLIW”) microprocessor, a processor implementing a combination of instruction sets, or any other processor device, such as a digital signal processor, for example. In at least one embodiment, processor 1802 may be coupled to a processor bus 1810 that may transmit data signals between processor 1802 and other components in computer system 1800.

In at least one embodiment, processor 1802 may include, without limitation, a Level 1 (“L1”) internal cache memory (“cache”) 1804. In at least one embodiment, processor 1802 may have a single internal cache or multiple levels of internal cache. In at least one embodiment, cache memory may reside external to processor 1802. Other embodiments may also include a combination of both internal and external caches depending on particular implementation and needs. In at least one embodiment, a register file 1806 may store different types of data in various registers including, without limitation, integer registers, floating point registers, status registers, and an instruction pointer register.

In at least one embodiment, execution unit 1808, including, without limitation, logic to perform integer and floating point operations, also resides in processor 1802. In at least one embodiment, processor 1802 may also include a microcode (“ucode”) read only memory (“ROM”) that stores microcode for certain macro instructions. In at least one embodiment, execution unit 1808 may include logic to handle a packed instruction set 1809. In at least one embodiment, by including packed instruction set 1809 in an instruction set of a general-purpose processor, along with associated circuitry to execute instructions, operations used by many multimedia applications may be performed using packed data in processor 1802. In at least one embodiment, many multimedia applications may be accelerated and executed more efficiently by using a full width of a processor's data bus for performing operations on packed data, which may eliminate a need to transfer smaller units of data across that processor's data bus to perform one or more operations one data element at a time.

In at least one embodiment, execution unit 1808 may also be used in microcontrollers, embedded processors, graphics devices, DSPs, and other types of logic circuits. In at least one embodiment, computer system 1800 may include, without limitation, a memory 1820. In at least one embodiment, memory 1820 may be a Dynamic Random Access Memory (“DRAM”) device, a Static Random Access Memory (“SRAM”) device, a flash memory device, or another memory device. In at least one embodiment, memory 1820 may store instruction(s) 1819 and/or data 1821 represented by data signals that may be executed by processor 1802.

In at least one embodiment, a system logic chip may be coupled to processor bus 1810 and memory 1820. In at least one embodiment, a system logic chip may include, without limitation, a memory controller hub (“MCH”) 1816, and processor 1802 may communicate with MCH 1816 via processor bus 1810. In at least one embodiment, MCH 1816 may provide a high bandwidth memory path 1818 to memory 1820 for instruction and data storage and for storage of graphics commands, data and textures. In at least one embodiment, MCH 1816 may direct data signals between processor 1802, memory 1820, and other components in computer system 1800 and to bridge data signals between processor bus 1810, memory 1820, and a system I/O interface 1822. In at least one embodiment, a system logic chip may provide a graphics port for coupling to a graphics controller. In at least one embodiment, MCH 1816 may be coupled to memory 1820 through high bandwidth memory path 1818 and a graphics/video card 1812 may be coupled to MCH 1816 through an Accelerated Graphics Port (“AGP”) interconnect 1814.

In at least one embodiment, computer system 1800 may use system I/O interface 1822 as a proprietary hub interface bus to couple MCH 1816 to an I/O controller hub (“ICH”) 1830. In at least one embodiment, ICH 1830 may provide direct connections to some I/O devices via a local I/O bus. In at least one embodiment, a local I/O bus may include, without limitation, a high-speed I/O bus for connecting peripherals to memory 1820, a chipset, and processor 1802. Examples may include, without limitation, an audio controller 1829, a firmware hub (“flash BIOS”) 1828, a wireless transceiver 1826, a data storage 1824, a legacy I/O controller 1823 containing user input and keyboard interfaces 1825, a serial expansion port 1827, such as a USB port, and a network controller 1834. In at least one embodiment, data storage 1824 may comprise a hard disk drive, a floppy disk drive, a CD-ROM device, a flash memory device, or other mass storage device.

In at least one embodiment, FIG. 18 illustrates a system, which includes interconnected hardware devices or “chips”, whereas in other embodiments, FIG. 18 may illustrate an exemplary SoC. In at least one embodiment, devices illustrated in FIG. 18 may be interconnected with proprietary interconnects, standardized interconnects (e.g., PCIe) or some combination thereof. In at least one embodiment, one or more components of computer system 1800 are interconnected using compute express link (CXL) interconnects.

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, inference and/or training logic 115 may be used in system FIG. 18 for inferencing or predicting operations based, at least in part, on weight parameters calculated using neural network training operations, neural network functions and/or architectures, or neural network use cases described herein.

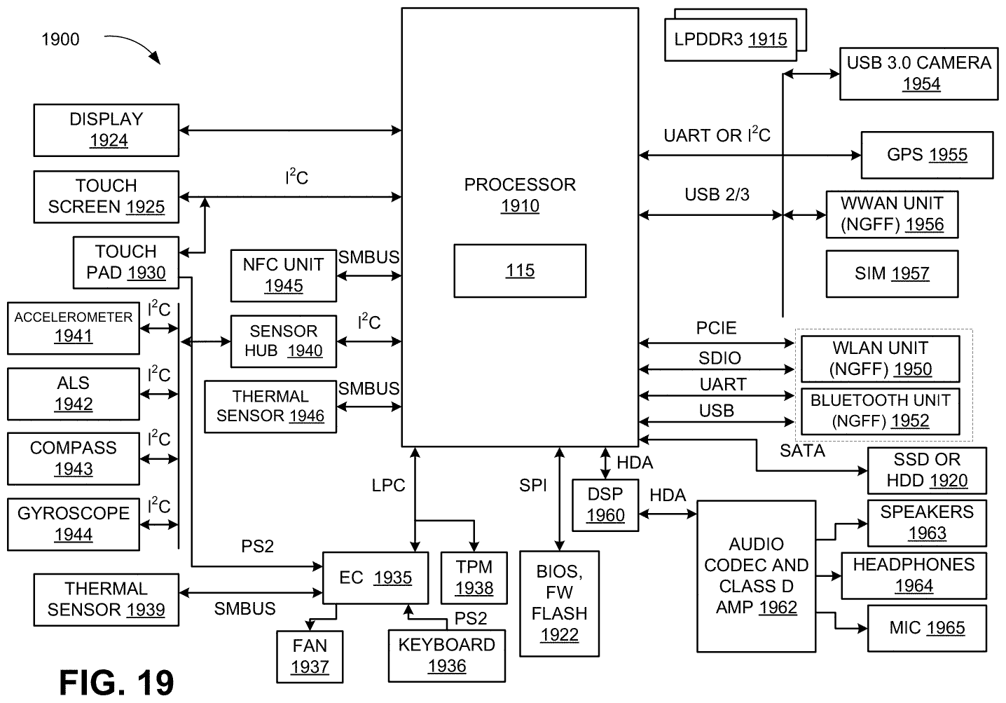

FIG. 19 is a block diagram illustrating an electronic device 1900 for utilizing a processor 1910, according to at least one embodiment. In at least one embodiment, electronic device 1900 may be, for example and without limitation, a notebook, a tower server, a rack server, a blade server, a laptop, a desktop, a tablet, a mobile device, a phone, an embedded computer, or any other suitable electronic device.

In at least one embodiment, electronic device 1900 may include, without limitation, processor 1910 communicatively coupled to any suitable number or kind of components, peripherals, modules, or devices. In at least one embodiment, processor 1910 is coupled using a bus or interface, such as a I²C bus, a System Management Bus (“SMBus”), a Low Pin Count (LPC) bus, a Serial Peripheral Interface (“SPI”), a High Definition Audio (“HDA”) bus, a Serial Advance Technology Attachment (“SATA”) bus, a Universal Serial Bus (“USB”) (versions 1, 2, 3, etc.), or a Universal Asynchronous Receiver/Transmitter (“UART”) bus. In at least one embodiment, FIG. 19 illustrates a system, which includes interconnected hardware devices or “chips”, whereas in other embodiments, FIG. 19 may illustrate an exemplary SoC. In at least one embodiment, devices illustrated in FIG. 19 may be interconnected with proprietary interconnects, standardized interconnects (e.g., PCIe) or some combination thereof. In at least one embodiment, one or more components of FIG. 19 are interconnected using compute express link (CXL) interconnects.

In at least one embodiment, FIG. 19 may include a display 1924, a touch screen 1925, a touch pad 1930, a Near Field Communications unit (“NFC”) 1945, a sensor hub 1940, a thermal sensor 1946, an Express Chipset (“EC”) 1935, a Trusted Platform Module (“TPM”) 1938, BIOS/firmware/flash memory (“BIOS, FW Flash”) 1922, a DSP 1960, a drive 1920 such as a Solid State Disk (“SSD”) or a Hard Disk Drive (“HDD”), a wireless local area network unit (“WLAN”) 1950, a Bluetooth unit 1952, a Wireless Wide Area Network unit (“WWAN”) 1956, a Global Positioning System (GPS) unit 1955, a camera (“USB 3.0 camera”) 1954 such as a USB 3.0 camera, and/or a Low Power Double Data Rate (“LPDDR”) memory unit (“LPDDR3”) 1915 implemented in, for example, an LPDDR3 standard. These components may each be implemented in any suitable manner.

In at least one embodiment, other components may be communicatively coupled to processor 1910 through components described herein. In at least one embodiment, an accelerometer 1941, an ambient light sensor (“ALS”) 1942, a compass 1943, and a gyroscope 1944 may be communicatively coupled to sensor hub 1940. In at least one embodiment, a thermal sensor 1939, a fan 1937, a keyboard 1936, and touch pad 1930 may be communicatively coupled to EC 1935. In at least one embodiment, speakers 1963, headphones 1964, and a microphone (“mic”) 1965 may be communicatively coupled to an audio unit (“audio codec and class D amp”) 1962, which may in turn be communicatively coupled to DSP 1960. In at least one embodiment, audio unit 1962 may include, for example and without limitation, an audio coder/decoder (“codec”) and a class D amplifier. In at least one embodiment, a SIM card (“SIM”) 1957 may be communicatively coupled to WWAN unit 1956. In at least one embodiment, components such as WLAN unit 1950 and Bluetooth unit 1952, as well as WWAN unit 1956 may be implemented in a Next Generation Form Factor (“NGFF”).

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, inference and/or training logic 115 may be used in system FIG. 1 for inferencing or predicting operations based, at least in part, on weight parameters calculated using neural network training operations, neural network functions and/or architectures, or neural network use cases described herein.

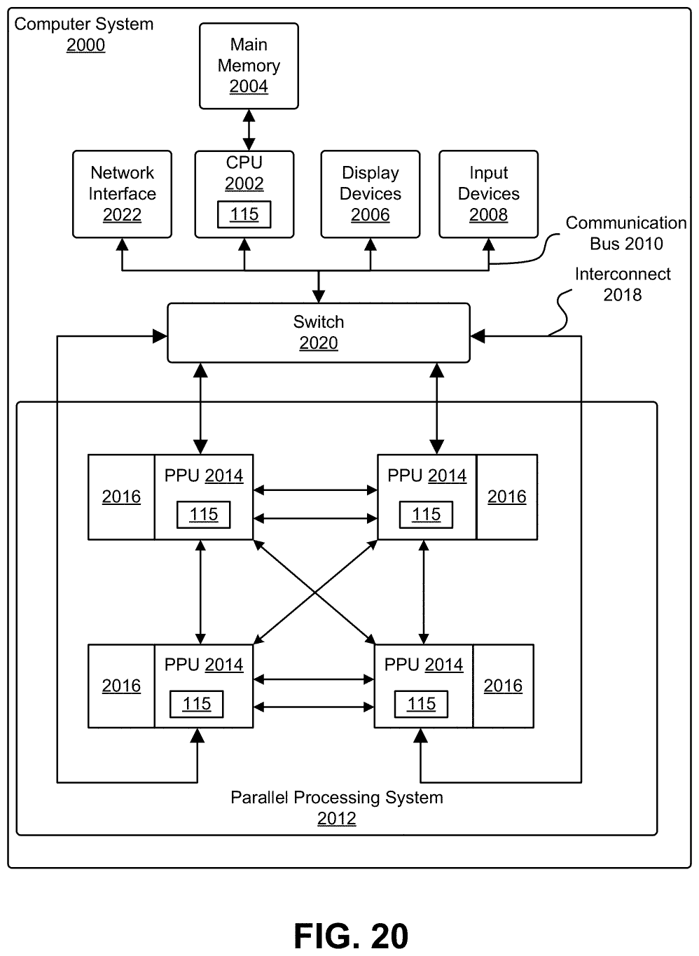

FIG. 20 illustrates a computer system 2000, according to at least one embodiment. In at least one embodiment, computer system 2000 is configured to implement various processes and methods described throughout this disclosure.

In at least one embodiment, computer system 2000 comprises, without limitation, at least one central processing unit (“CPU”) 2002 that is connected to a communication bus 2010 implemented using any suitable protocol, such as PCI (“Peripheral Component Interconnect”), peripheral component interconnect express (“PCI-Express”), AGP (“Accelerated Graphics Port”), HyperTransport, or any other bus or point-to-point communication protocol(s). In at least one embodiment, computer system 2000 includes, without limitation, a main memory 2004 and control logic (e.g., implemented as hardware, software, or a combination thereof) and data are stored in main memory 2004, which may take form of random access memory (“RAM”). In at least one embodiment, a network interface subsystem (“network interface”) 2022 provides an interface to other computing devices and networks for receiving data from and transmitting data to other systems with computer system 2000.

In at least one embodiment, computer system 2000, in at least one embodiment, includes, without limitation, input devices 2008, a parallel processing system 2012, and display devices 2006 that can be implemented using a conventional cathode ray tube (“CRT”), a liquid crystal display (“LCD”), a light emitting diode (“LED”) display, a plasma display, or other suitable display technologies. In at least one embodiment, user input is received from input devices 2008 such as keyboard, mouse, touchpad, microphone, etc. In at least one embodiment, each module described herein can be situated on a single semiconductor platform to form a processing system.

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, inference and/or training logic 115 may be used in system FIG. 20 for inferencing or predicting operations based, at least in part, on weight parameters calculated using neural network training operations, neural network functions and/or architectures, or neural network use cases described herein.

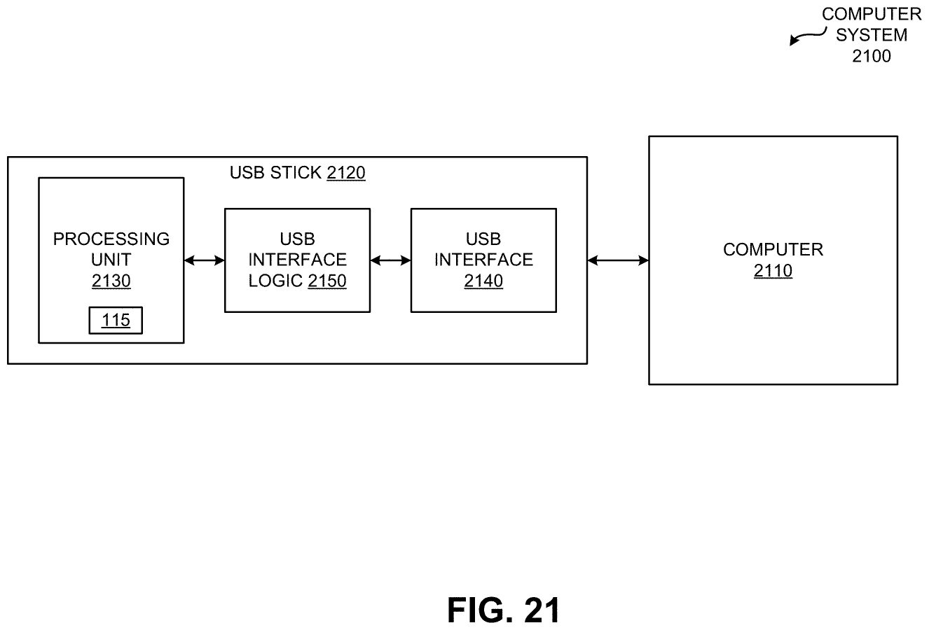

FIG. 21 illustrates a computer system 2100, according to at least one embodiment. In at least one embodiment, computer system 2100 includes, without limitation, a computer 2110 and a USB stick 2120. In at least one embodiment, computer 2110 may include, without limitation, any number and type of processor(s) (not shown) and a memory (not shown). In at least one embodiment, computer 2110 includes, without limitation, a server, a cloud instance, a laptop, and a desktop computer.

In at least one embodiment, USB stick 2120 includes, without limitation, a processing unit 2130, a USB interface 2140, and USB interface logic 2150. In at least one embodiment, processing unit 2130 may be any instruction execution system, apparatus, or device capable of executing instructions. In at least one embodiment, processing unit 2130 may include, without limitation, any number and type of processing cores (not shown). In at least one embodiment, processing unit 2130 comprises an application specific integrated circuit (“ASIC”) that is optimized to perform any amount and type of operations associated with machine learning. For instance, in at least one embodiment, processing unit 2130 is a tensor processing unit (“TPC”) that is optimized to perform machine learning inference operations. In at least one embodiment, processing unit 2130 is a vision processing unit (“VPU”) that is optimized to perform machine vision and machine learning inference operations.

In at least one embodiment, USB interface 2140 may be any type of USB connector or USB socket. For instance, in at least one embodiment, USB interface 2140 is a USB 3.0 Type-C socket for data and power. In at least one embodiment, USB interface 2140 is a USB 3.0 Type-A connector. In at least one embodiment, USB interface logic 2150 may include any amount and type of logic that enables processing unit 2130 to interface with devices (e.g., computer 2110) via USB interface 2140.

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, inference and/or training logic 115 may be used in system FIG. 21 for inferencing or predicting operations based, at least in part, on weight parameters calculated using neural network training operations, neural network functions and/or architectures, or neural network use cases described herein.

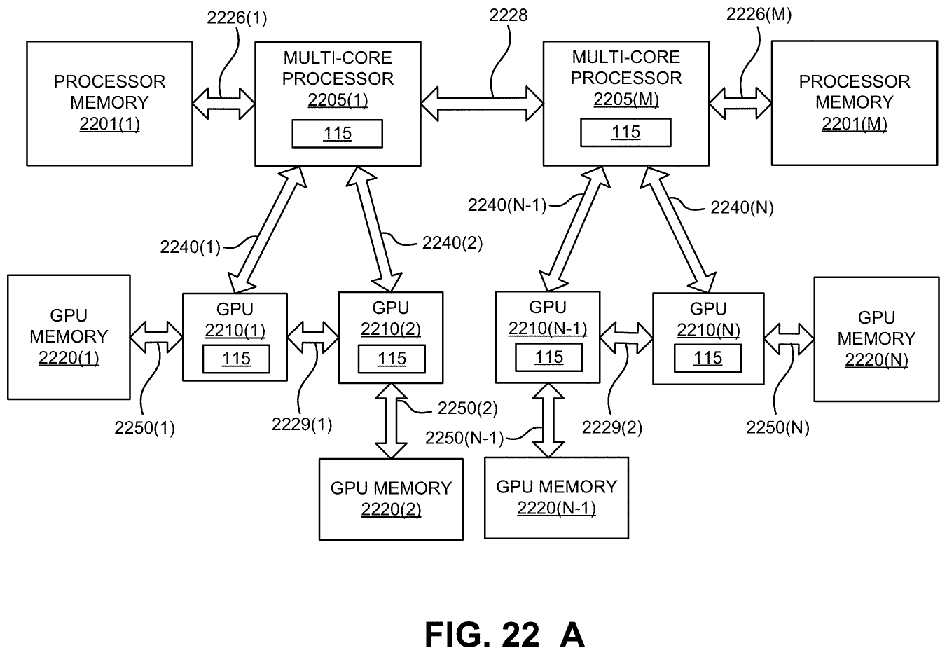

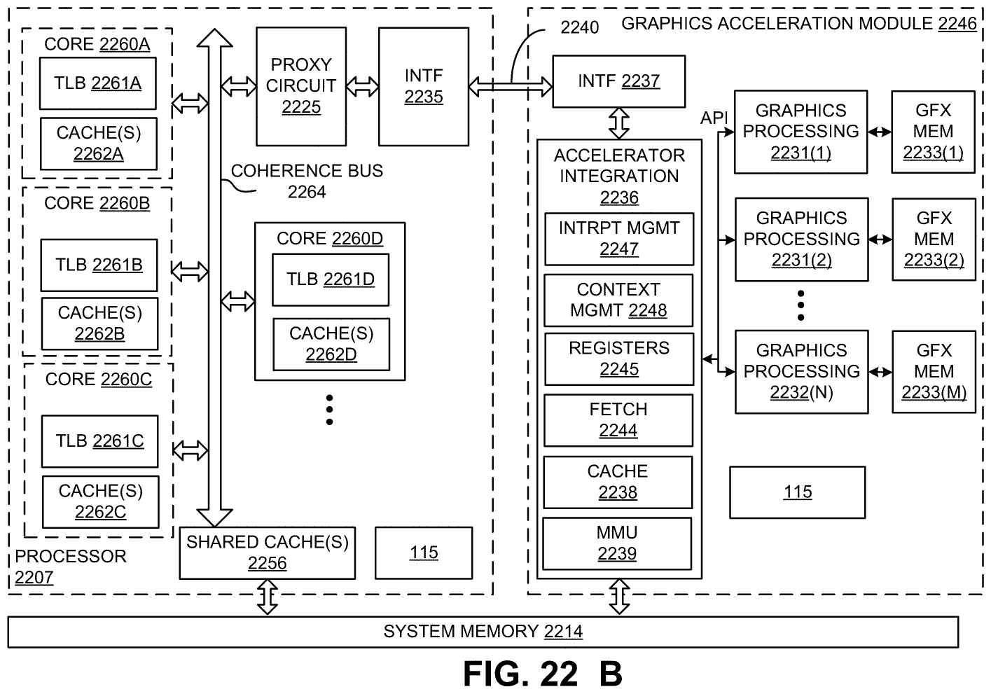

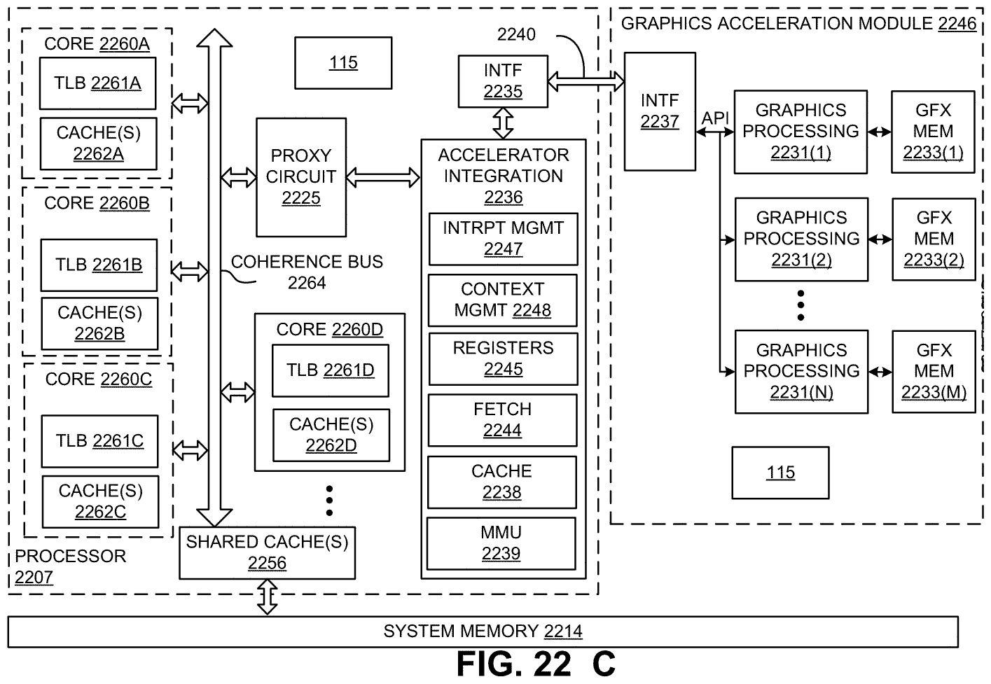

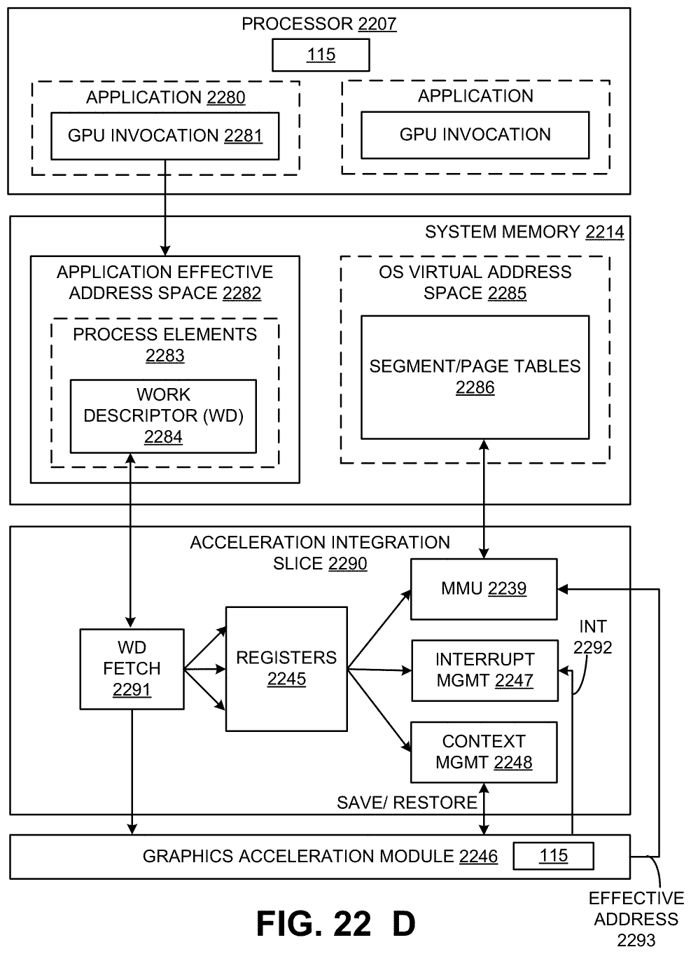

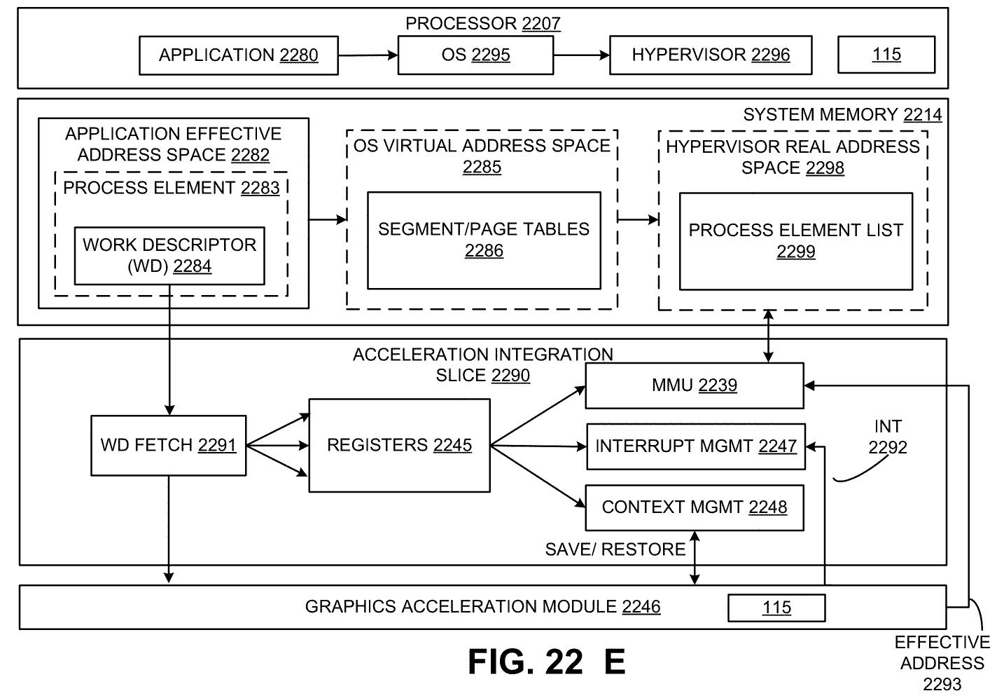

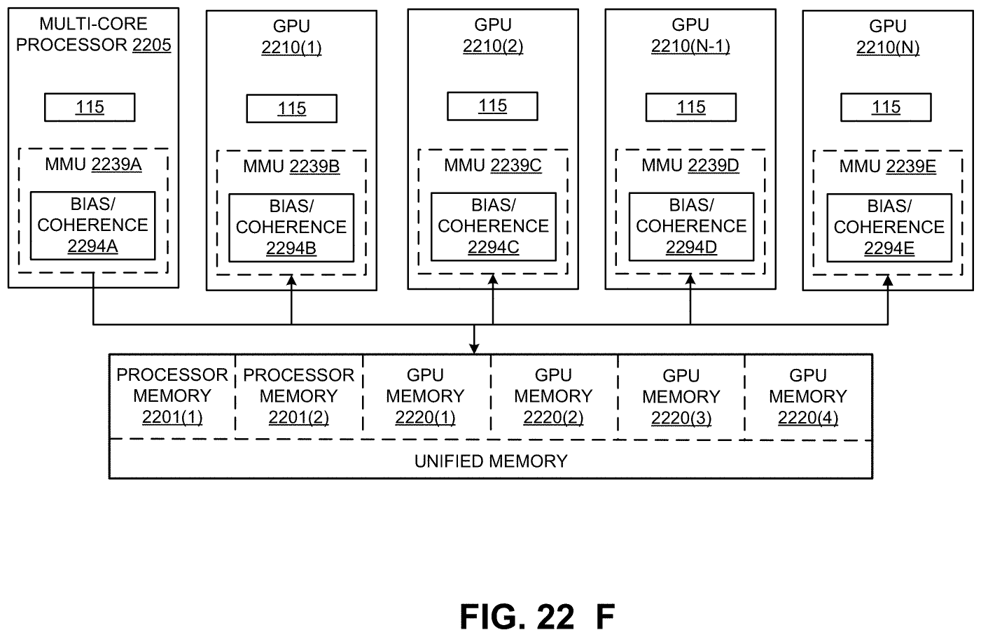

FIG. 22A illustrates an exemplary architecture in which a plurality of GPUs 2210(1)-2210(N) is communicatively coupled to a plurality of multi-core processors 2205(1)-2205(M) over high-speed links 2240(1)-2240(N) (e.g., buses, point-to-point interconnects, etc.). In at least one embodiment, high-speed links 2240(1)-2240(N) support a communication throughput of 4 GB/s, 30 GB/s, 80 GB/s or higher. In at least one embodiment, various interconnect protocols may be used including, but not limited to, PCIe 4.0 or 5.0 and NVLink 2.0. In various figures, “N” and “M” represent positive integers, values of which may be different from figure to figure.

In addition, and in at least one embodiment, two or more of GPUs 2210 are interconnected over high-speed links 2229(1)-2229(2), which may be implemented using similar or different protocols/links than those used for high-speed links 2240(1)-2240(N). Similarly, two or more of multi-core processors 2205 may be connected over a high-speed link 2228 which may be symmetric multi-processor (SMP) buses operating at 20 GB/s, 30 GB/s, 120 GB/s or higher. Alternatively, all communication between various system components shown in FIG. 22A may be accomplished using similar protocols/links (e.g., over a common interconnection fabric).

In at least one embodiment, each multi-core processor 2205 is communicatively coupled to a processor memory 2201(1)-2201(M), via memory interconnects 2226(1)-2226(M), respectively, and each GPU 2210(1)-2210(N) is communicatively coupled to GPU memory 2220(1)-2220(N) over GPU memory interconnects 2250(1)-2250(N), respectively. In at least one embodiment, memory interconnects 2226 and 2250 may utilize similar or different memory access technologies. By way of example, and not limitation, processor memories 2201(1)-2201(M) and GPU memories 2220 may be volatile memories such as dynamic random access memories (DRAMs) (including stacked DRAMs), Graphics DDR SDRAM (GDDR) (e.g., GDDR5, GDDR6), or High Bandwidth Memory (HBM) and/or may be non-volatile memories such as 3D XPoint or Nano-Ram. In at least one embodiment, some portion of processor memories 2201 may be volatile memory and another portion may be non-volatile memory (e.g., using a two-level memory (2LM) hierarchy).

As described herein, although various multi-core processors 2205 and GPUs 2210 may be physically coupled to a particular memory 2201, 2220, respectively, and/or a unified memory architecture may be implemented in which a virtual system address space (also referred to as “effective address” space) is distributed among various physical memories. For example, processor memories 2201(1)-2201(M) may each comprise 64 GB of system memory address space and GPU memories 2220(1)-2220(N) may each comprise 32 GB of system memory address space resulting in a total of 256 GB addressable memory when M=2 and N=4. Other values for N and M are possible.

FIG. 22B illustrates additional details for an interconnection between a multi-core processor 2207 and a graphics acceleration module 2246 in accordance with one exemplary embodiment. In at least one embodiment, graphics acceleration module 2246 may include one or more GPU chips integrated on a line card which is coupled to processor 2207 via high-speed link 2240 (e.g., a PCIe bus, NVLink, etc.). In at least one embodiment, graphics acceleration module 2246 may alternatively be integrated on a package or chip with processor 2207.

In at least one embodiment, processor 2207 includes a plurality of cores 2260A-2260D, each with a translation lookaside buffer (“TLB”) 2261A-2261D and one or more caches 2262A-2262D. In at least one embodiment, cores 2260A-2260D may include various other components for executing instructions and processing data that are not illustrated. In at least one embodiment, caches 2262A-2262D may comprise Level 1 (L1) and Level 2 (L2) caches. In addition, one or more shared caches 2256 may be included in caches 2262A-2262D and shared by sets of cores 2260A-2260D. For example, one embodiment of processor 2207 includes 24 cores, each with its own L1 cache, twelve shared L2 caches, and twelve shared L3 caches. In this embodiment, one or more L2 and L3 caches are shared by two adjacent cores. In at least one embodiment, processor 2207 and graphics acceleration module 2246 connect with system memory 2214, which may include processor memories 2201(1)-2201(M) of FIG. 22A.

In at least one embodiment, coherency is maintained for data and instructions stored in various caches 2262A-2262D, 2256 and system memory 2214 via inter-core communication over a coherence bus 2264. In at least one embodiment, for example, each cache may have cache coherency logic/circuitry associated therewith to communicate to over coherence bus 2264 in response to detected reads or writes to particular cache lines. In at least one embodiment, a cache snooping protocol is implemented over coherence bus 2264 to snoop cache accesses.

In at least one embodiment, a proxy circuit 2225 communicatively couples graphics acceleration module 2246 to coherence bus 2264, allowing graphics acceleration module 2246 to participate in a cache coherence protocol as a peer of cores 2260A-2260D. In particular, in at least one embodiment, an interface 2235 provides connectivity to proxy circuit 2225 over high-speed link 2240 and an interface 2237 connects graphics acceleration module 2246 to high-speed link 2240.

In at least one embodiment, an accelerator integration circuit 2236 provides cache management, memory access, context management, and interrupt management services on behalf of a plurality of graphics processing engines 2231(1)-2231(N) of graphics acceleration module 2246. In at least one embodiment, graphics processing engines 2231(1)-2231(N) may each comprise a separate GPU. In at least one embodiment, graphics processing engines 2231(1)-2231(N) alternatively may comprise different types of graphics processing engines within a GPU, such as graphics execution units, media processing engines (e.g., video encoders/decoders), samplers, and blit engines. In at least one embodiment, graphics acceleration module 2246 may be a GPU with a plurality of graphics processing engines 2231(1)-2231(N) or graphics processing engines 2231(1)-2231(N) may be individual GPUs integrated on a common package, line card, or chip.

In at least one embodiment, accelerator integration circuit 2236 includes a memory management unit (MMU) 2239 for performing various memory management functions such as virtual-to-physical memory translations (also referred to as effective-to-real memory translations) and memory access protocols for accessing system memory 2214. In at least one embodiment, MMU 2239 may also include a translation lookaside buffer (TLB) (not shown) for caching virtual/effective to physical/real address translations. In at least one embodiment, a cache 2238 can store commands and data for efficient access by graphics processing engines 2231(1)-2231(N). In at least one embodiment, data stored in cache 2238 and graphics memories 2233(1)-2233(M) is kept coherent with core caches 2262A-2262D, 2256 and system memory 2214, possibly using a fetch unit 2244. As mentioned, this may be accomplished via proxy circuit 2225 on behalf of cache 2238 and memories 2233(1)-2233(M) (e.g., sending updates to cache 2238 related to modifications/accesses of cache lines on processor caches 2262A-2262D, 2256 and receiving updates from cache 2238).

In at least one embodiment, a set of registers 2245 store context data for threads executed by graphics processing engines 2231(1)-2231(N) and a context management circuit 2248 manages thread contexts. For example, context management circuit 2248 may perform save and restore operations to save and restore contexts of various threads during contexts switches (e.g., where a first thread is saved and a second thread is stored so that a second thread can be execute by a graphics processing engine). For example, on a context switch, context management circuit 2248 may store current register values to a designated region in memory (e.g., identified by a context pointer). It may then restore register values when returning to a context. In at least one embodiment, an interrupt management circuit 2247 receives and processes interrupts received from system devices.

In at least one embodiment, virtual/effective addresses from a graphics processing engine 2231 are translated to real/physical addresses in system memory 2214 by MMU 2239. In at least one embodiment, accelerator integration circuit 2236 supports multiple (e.g., 4, 8, 16) graphics accelerator modules 2246 and/or other accelerator devices. In at least one embodiment, graphics accelerator module 2246 may be dedicated to a single application executed on processor 2207 or may be shared between multiple applications. In at least one embodiment, a virtualized graphics execution environment is presented in which resources of graphics processing engines 2231(1)-2231(N) are shared with multiple applications or virtual machines (VMs). In at least one embodiment, resources may be subdivided into “slices” which are allocated to different VMs and/or applications based on processing requirements and priorities associated with VMs and/or applications.

In at least one embodiment, accelerator integration circuit 2236 performs as a bridge to a system for graphics acceleration module 2246 and provides address translation and system memory cache services. In addition, in at least one embodiment, accelerator integration circuit 2236 may provide virtualization facilities for a host processor to manage virtualization of graphics processing engines 2231(1)-2231(N), interrupts, and memory management.

In at least one embodiment, because hardware resources of graphics processing engines 2231(1)-2231(N) are mapped explicitly to a real address space seen by host processor 2207, any host processor can address these resources directly using an effective address value. In at least one embodiment, one function of accelerator integration circuit 2236 is physical separation of graphics processing engines 2231(1)-2231(N) so that they appear to a system as independent units.

In at least one embodiment, one or more graphics memories 2233(1)-2233(M) are coupled to each of graphics processing engines 2231(1)-2231(N), respectively and N=M. In at least one embodiment, graphics memories 2233(1)-2233(M) store instructions and data being processed by each of graphics processing engines 2231(1)-2231(N). In at least one embodiment, graphics memories 2233(1)-2233(M) may be volatile memories such as DRAMs (including stacked DRAMs), GDDR memory (e.g., GDDR5, GDDR6), or HBM, and/or may be non-volatile memories such as 3D XPoint or Nano-Ram.

In at least one embodiment, to reduce data traffic over high-speed link 2240, biasing techniques can be used to ensure that data stored in graphics memories 2233(1)-2233(M) is data that will be used most frequently by graphics processing engines 2231(1)-2231(N) and not used by cores 2260A-2260D (at least not frequently). Similarly, in at least one embodiment, a biasing mechanism attempts to keep data needed by cores (and not graphics processing engines 2231(1)-2231(N)) within caches 2262A-2262D, 2256 and system memory 2214.

FIG. 22C illustrates another exemplary embodiment in which accelerator integration circuit 2236 is integrated within processor 2207. In this embodiment, graphics processing engines 2231(1)-2231(N) communicate directly over high-speed link 2240 to accelerator integration circuit 2236 via interface 2237 and interface 2235 (which, again, may be any form of bus or interface protocol). In at least one embodiment, accelerator integration circuit 2236 may perform similar operations as those described with respect to FIG. 22B, but potentially at a higher throughput given its close proximity to coherence bus 2264 and caches 2262A-2262D, 2256. In at least one embodiment, an accelerator integration circuit supports different programming models including a dedicated-process programming model (no graphics acceleration module virtualization) and shared programming models (with virtualization), which may include programming models which are controlled by accelerator integration circuit 2236 and programming models which are controlled by graphics acceleration module 2246.

In at least one embodiment, graphics processing engines 2231(1)-2231(N) are dedicated to a single application or process under a single operating system. In at least one embodiment, a single application can funnel other application requests to graphics processing engines 2231(1)-2231(N), providing virtualization within a VM/partition.

In at least one embodiment, graphics processing engines 2231(1)-2231(N), may be shared by multiple VM/application partitions. In at least one embodiment, shared models may use a system hypervisor to virtualize graphics processing engines 2231(1)-2231(N) to allow access by each operating system. In at least one embodiment, for single-partition systems without a hypervisor, graphics processing engines 2231(1)-2231(N) are owned by an operating system. In at least one embodiment, an operating system can virtualize graphics processing engines 2231(1)-2231(N) to provide access to each process or application.

In at least one embodiment, graphics acceleration module 2246 or an individual graphics processing engine 2231(1)-2231(N) selects a process element using a process handle. In at least one embodiment, process elements are stored in system memory 2214 and are addressable using an effective address to real address translation technique described herein. In at least one embodiment, a process handle may be an implementation-specific value provided to a host process when registering its context with graphics processing engine 2231(1)-2231(N) (that is, calling system software to add a process element to a process element linked list). In at least one embodiment, a lower 16-bits of a process handle may be an offset of a process element within a process element linked list.

FIG. 22D illustrates an exemplary accelerator integration slice 2290. In at least one embodiment, a “slice” comprises a specified portion of processing resources of accelerator integration circuit 2236. In at least one embodiment, an application is effective address space 2282 within system memory 2214 stores process elements 2283. In at least one embodiment, process elements 2283 are stored in response to GPU invocations 2281 from applications 2280 executed on processor 2207. In at least one embodiment, a process element 2283 contains process state for corresponding application 2280. In at least one embodiment, a work descriptor (WD) 2284 contained in process element 2283 can be a single job requested by an application or may contain a pointer to a queue of jobs. In at least one embodiment, WD 2284 is a pointer to a job request queue in an application's effective address space 2282.

In at least one embodiment, graphics acceleration module 2246 and/or individual graphics processing engines 2231(1)-2231(N) can be shared by all or a subset of processes in a system. In at least one embodiment, an infrastructure for setting up process states and sending a WD 2284 to a graphics acceleration module 2246 to start a job in a virtualized environment may be included.

In at least one embodiment, a dedicated-process programming model is implementation-specific. In at least one embodiment, in this model, a single process owns graphics acceleration module 2246 or an individual graphics processing engine 2231. In at least one embodiment, when graphics acceleration module 2246 is owned by a single process, a hypervisor initializes accelerator integration circuit 2236 for an owning partition and an operating system initializes accelerator integration circuit 2236 for an owning process when graphics acceleration module 2246 is assigned.

In at least one embodiment, in operation, a WD fetch unit 2291 in accelerator integration slice 2290 fetches next WD 2284, which includes an indication of work to be done by one or more graphics processing engines of graphics acceleration module 2246. In at least one embodiment, data from WD 2284 may be stored in registers 2245 and used by MMU 2239, interrupt management circuit 2247 and/or context management circuit 2248 as illustrated. For example, one embodiment of MMU 2239 includes segment/page walk circuitry for accessing segment/page tables 2286 within an OS virtual address space 2285. In at least one embodiment, interrupt management circuit 2247 may process interrupt events 2292 received from graphics acceleration module 2246. In at least one embodiment, when performing graphics operations, an effective address 2293 generated by a graphics processing engine 2231(1)-2231(N) is translated to a real address by MMU 2239.

In at least one embodiment, registers 2245 are duplicated for each graphics processing engine 2231(1)-2231(N) and/or graphics acceleration module 2246 and may be initialized by a hypervisor or an operating system. In at least one embodiment, each of these duplicated registers may be included in an accelerator integration slice 2290. Exemplary registers that may be initialized by a hypervisor are shown in Table 1.

TABLE 1
Hypervisor Initialized Registers

| Register # | Description |
|---|---|
| 1 | Slice Control Register |
| 2 | Real Address (RA) Scheduled Processes Area Pointer |
| 3 | Authority Mask Override Register |
| 4 | Interrupt Vector Table Entry Offset |
| 5 | Interrupt Vector Table Entry Limit |
| 6 | State Register |
| 7 | Logical Partition ID |
| 8 | Real address (RA) Hypervisor Accelerator |
| | Utilization Record Pointer |
| 9 | Storage Description Register |

Exemplary registers that may be initialized by an operating system are shown in Table 2.

TABLE 2
Operating System Initialized Registers

| Register # | Description |
|---|---|
| 1 | Process and Thread Identification |
| 2 | Effective Address (EA) Context Save/Restore Pointer |
| 3 | Virtual Address (VA) Accelerator Utilization Record Pointer |
| 4 | Virtual Address (VA) Storage Segment Table Pointer |
| 5 | Authority Mask |
| 6 | Work descriptor |

In at least one embodiment, each WD 2284 is specific to a particular graphics acceleration module 2246 and/or graphics processing engines 2231(1)-2231(N). In at least one embodiment, it contains all information required by a graphics processing engine 2231(1)-2231(N) to do work, or it can be a pointer to a memory location where an application has set up a command queue of work to be completed.

FIG. 22E illustrates additional details for one exemplary embodiment of a shared model. This embodiment includes a hypervisor real address space 2298 in which a process element list 2299 is stored. In at least one embodiment, hypervisor real address space 2298 is accessible via a hypervisor 2296 which virtualizes graphics acceleration module engines for operating system 2295.

In at least one embodiment, shared programming models allow for all or a subset of processes from all or a subset of partitions in a system to use a graphics acceleration module 2246. In at least one embodiment, there are two programming models where graphics acceleration module 2246 is shared by multiple processes and partitions, namely time-sliced shared and graphics directed shared.

In at least one embodiment, in this model, system hypervisor 2296 owns graphics acceleration module 2246 and makes its function available to all operating systems 2295. In at least one embodiment, for a graphics acceleration module 2246 to support virtualization by system hypervisor 2296, graphics acceleration module 2246 may adhere to certain requirements, such as (1) an application's job request must be autonomous (that is, state does not need to be maintained between jobs), or graphics acceleration module 2246 must provide a context save and restore mechanism, (2) an application's job request is guaranteed by graphics acceleration module 2246 to complete in a specified amount of time, including any translation faults, or graphics acceleration module 2246 provides an ability to preempt processing of a job, and (3) graphics acceleration module 2246 must be guaranteed fairness between processes when operating in a directed shared programming model.

In at least one embodiment, application 2280 is required to make an operating system 2295 system call with a graphics acceleration module type, a work descriptor (WD), an authority mask register (AMR) value, and a context save/restore area pointer (CSRP). In at least one embodiment, graphics acceleration module type describes a targeted acceleration function for a system call. In at least one embodiment, graphics acceleration module type may be a system-specific value. In at least one embodiment, WD is formatted specifically for graphics acceleration module 2246 and can be in a form of a graphics acceleration module 2246 command, an effective address pointer to a user-defined structure, an effective address pointer to a queue of commands, or any other data structure to describe work to be done by graphics acceleration module 2246.

In at least one embodiment, an AMR value is an AMR state to use for a current process. In at least one embodiment, a value passed to an operating system is similar to an application setting an AMR. In at least one embodiment, if accelerator integration circuit 2236 (not shown) and graphics acceleration module 2246 implementations do not support a User Authority Mask Override Register (UAMOR), an operating system may apply a current UAMOR value to an AMR value before passing an AMR in a hypervisor call. In at least one embodiment, hypervisor 2296 may optionally apply a current Authority Mask Override Register (AMOR) value before placing an AMR into process element 2283. In at least one embodiment, CSRP is one of registers 2245 containing an effective address of an area in an application's effective address space 2282 for graphics acceleration module 2246 to save and restore context state. In at least one embodiment, this pointer is optional if no state is required to be saved between jobs or when a job is preempted. In at least one embodiment, context save/restore area may be pinned system memory.

Upon receiving a system call, operating system 2295 may verify that application 2280 has registered and been given authority to use graphics acceleration module 2246. In at least one embodiment, operating system 2295 then calls hypervisor 2296 with information shown in Table

TABLE 3
OS to Hypervisor Call Parameters

| Parameter # | Description |
|---|---|
| 1 | A work descriptor (WD) |
| 2 | An Authority Mask Register (AMR) |
| | value (potentially masked) |
| 3 | An effective address (EA) Context |
| | Save/Restore Area Pointer (CSRP) |
| 4 | A process ID (PID) and optional thread ID (TID) |
| 5 | A virtual address (VA) accelerator |
| | utilization record pointer (AURP) |
| 6 | Virtual address of storage |
| | segment table pointer (SSTP) |
| 7 | A logical interrupt service number (LISN) |

In at least one embodiment, upon receiving a hypervisor call, hypervisor 2296 verifies that operating system 2295 has registered and been given authority to use graphics acceleration module 2246. In at least one embodiment, hypervisor 2296 then puts process element 2283 into a process element linked list for a corresponding graphics acceleration module 2246 type. In at least one embodiment, a process element may include information shown in Table 4.

TABLE 4
Process Element Information

| Element # | Description |
|---|---|
| 1 | A work descriptor (WD) |
| 2 | An Authority Mask Register (AMR) |
| | value (potentially masked). |
| 3 | An effective address (EA) Context |
| | Save/Restore Area Pointer (CSRP) |
| 4 | A process ID (PID) and optional thread ID (TID) |
| 5 | A virtual address (VA) accelerator |
| | utilization record pointer (AURP) |
| 6 | Virtual address of storage |
| | segment table pointer (SSTP) |
| 7 | A logical interrupt service number (LISN) |
| 8 | Interrupt vector table, derived from |
| | hypervisor call parameters |
| 9 | A state register (SR) value |
| 10 | A logical partition ID (LPID) |
| 11 | A real address (RA) hypervisor accelerator |
| | utilization record pointer |
| 12 | Storage Descriptor Register (SDR) |

In at least one embodiment, hypervisor initializes a plurality of accelerator integration slice 2290 registers 2245.

As illustrated in FIG. 22F, in at least one embodiment, a unified memory is used, addressable via a common virtual memory address space used to access physical processor memories 2201(1)-2201(N) and GPU memories 2220(1)-2220(N). In this implementation, operations executed on GPUs 2210(1)-2210(N) utilize a same virtual/effective memory address space to access processor memories 2201(1)-2201(M) and vice versa, thereby simplifying programmability. In at least one embodiment, a first portion of a virtual/effective address space is allocated to processor memory 2201(1), a second portion to second processor memory 2201(N), a third portion to GPU memory 2220(1), and so on. In at least one embodiment, an entire virtual/effective memory space (sometimes referred to as an effective address space) is thereby distributed across each of processor memories 2201 and GPU memories 2220, allowing any processor or GPU to access any physical memory with a virtual address mapped to that memory.

In at least one embodiment, bias/coherence management circuitry 2294A-2294E within one or more of MMUs 2239A-2239E ensures cache coherence between caches of one or more host processors (e.g., multi-core processors 2205) and GPUs 2210 and implements biasing techniques indicating physical memories in which certain types of data should be stored. In at least one embodiment, while multiple instances of bias/coherence management circuitry 2294A-2294E are illustrated in FIG. 22F, bias/coherence circuitry may be implemented within an MMU of one or more host processors (e.g., multi-core processors 2205) and/or within accelerator integration circuit 2236.

One embodiment allows GPU memories 2220 to be mapped as part of system memory, and accessed using shared virtual memory (SVM) technology, but without suffering performance drawbacks associated with full system cache coherence. In at least one embodiment, an ability for GPU memories 2220 to be accessed as system memory without onerous cache coherence overhead provides a beneficial operating environment for GPU offload. In at least one embodiment, this arrangement allows software of host processor (e.g., multi-core processors 2205) to setup operands and access computation results, without overhead of tradition I/O DMA data copies. In at least one embodiment, such traditional copies involve driver calls, interrupts and memory mapped I/O (MMIO) accesses that are all inefficient relative to simple memory accesses. In at least one embodiment, an ability to access GPU memories 2220 without cache coherence overheads can be critical to execution time of an offloaded computation. In at least one embodiment, in cases with substantial streaming write memory traffic, for example, cache coherence overhead can significantly reduce an effective write bandwidth seen by a GPU 2210. In at least one embodiment, efficiency of operand setup, efficiency of results access, and efficiency of GPU computation may play a role in determining effectiveness of a GPU offload.

In at least one embodiment, selection of GPU bias and host processor bias is driven by a bias tracker data structure. In at least one embodiment, a bias table may be used, for example, which may be a page-granular structure (e.g., controlled at a granularity of a memory page) that includes 1 or 2 bits per GPU-attached memory page. In at least one embodiment, a bias table may be implemented in a stolen memory range of one or more GPU memories 2220, with or without a bias cache in a GPU 2210 (e.g., to cache frequently/recently used entries of a bias table). Alternatively, in at least one embodiment, an entire bias table may be maintained within a GPU.

In at least one embodiment, a bias table entry associated with each access to a GPU attached memory 2220 is accessed prior to actual access to a GPU memory, causing following operations. In at least one embodiment, local requests from a GPU 2210 that find their page in GPU bias are forwarded directly to a corresponding GPU memory 2220. In at least one embodiment, local requests from a GPU that find their page in host bias are forwarded to multi-core processors 2205 (e.g., over a high-speed link as described herein). In at least one embodiment, requests from multi-core processors 2205 that find a requested page in host processor bias complete a request like a normal memory read. Alternatively, requests directed to a GPU-biased page may be forwarded to a GPU 2210. In at least one embodiment, a GPU may then transition a page to a host processor bias if it is not currently using a page. In at least one embodiment, a bias state of a page can be changed either by a software-based mechanism, a hardware-assisted software-based mechanism, or, for a limited set of cases, a purely hardware-based mechanism.

In at least one embodiment, one mechanism for changing bias state employs an API call (e.g., OpenCL), which, in turn, calls a GPU's device driver which, in turn, sends a message (or enqueues a command descriptor) to a GPU directing it to change a bias state and, for some transitions, perform a cache flushing operation in a host. In at least one embodiment, a cache flushing operation is used for a transition from host processor (e.g., 2205) bias to GPU bias, but is not for an opposite transition.

In at least one embodiment, cache coherency is maintained by temporarily rendering GPU-biased pages uncacheable by host processor (e.g., 2205). In at least one embodiment, to access these pages, processor (e.g., 2205) may request access from GPU 2210, which may or may not grant access right away. In at least one embodiment, thus, to reduce communication between processor (e.g., 2205) and GPU 2210 it is beneficial to ensure that GPU-biased pages are those which are required by a GPU but not host processor (e.g., 2205) and vice versa.

Hardware structure(s) of inference and/or training logic 115 115 are used to perform one or more embodiments. Details regarding hardware structure(s) of inference and/or training logic 115 may be provided herein in conjunction with FIGS. 1A and/or 1B.

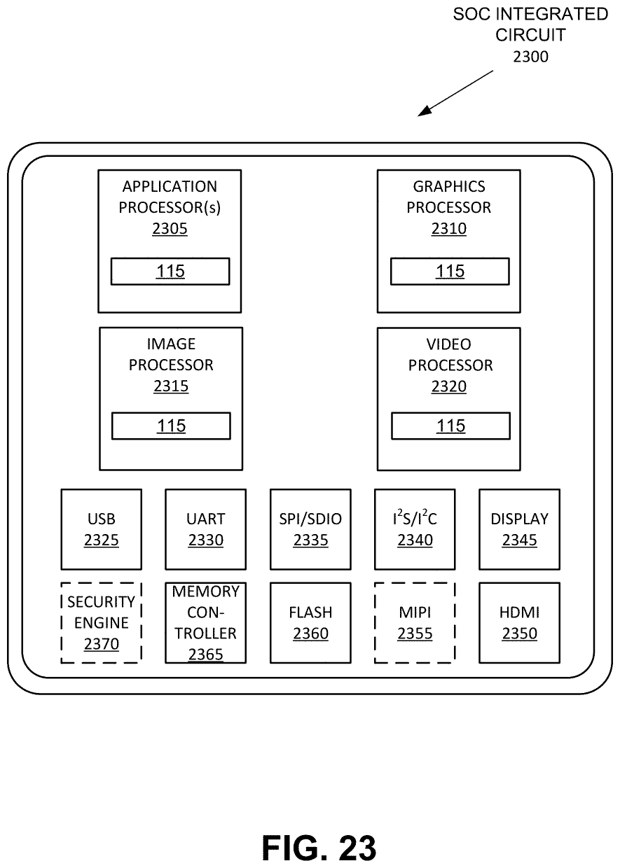

FIG. 23 illustrates exemplary integrated circuits and associated graphics processors that may be fabricated using one or more IP cores, according to various embodiments described herein. In addition to what is illustrated, other logic and circuits may be included in at least one embodiment, including additional graphics processors/cores, peripheral interface controllers, or general-purpose processor cores.

FIG. 23 is a block diagram illustrating an exemplary system on a chip integrated circuit 2300 that may be fabricated using one or more IP cores, according to at least one embodiment. In at least one embodiment, integrated circuit 2300 includes one or more application processor(s) 2305 (e.g., CPUs), at least one graphics processor 2310, and may additionally include an image processor 2315 and/or a video processor 2320, any of which may be a modular IP core. In at least one embodiment, integrated circuit 2300 includes peripheral or bus logic including a USB controller 2325, a UART controller 2330, an SPI/SDIO controller 2335, and an I²S/I²C controller 2340. In at least one embodiment, integrated circuit 2300 can include a display device 2345 coupled to one or more of a high-definition multimedia interface (HDMI) controller 2350 and a mobile industry processor interface (MIPI) display interface 2355. In at least one embodiment, storage may be provided by a flash memory subsystem 2360 including flash memory and a flash memory controller. In at least one embodiment, a memory interface may be provided via a memory controller 2365 for access to SDRAM or SRAM memory devices. In at least one embodiment, some integrated circuits additionally include an embedded security engine 2370.

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, inference and/or training logic 115 may be used in integrated circuit 2300 for inferencing or predicting operations based, at least in part, on weight parameters calculated using neural network training operations, neural network functions and/or architectures, or neural network use cases described herein.

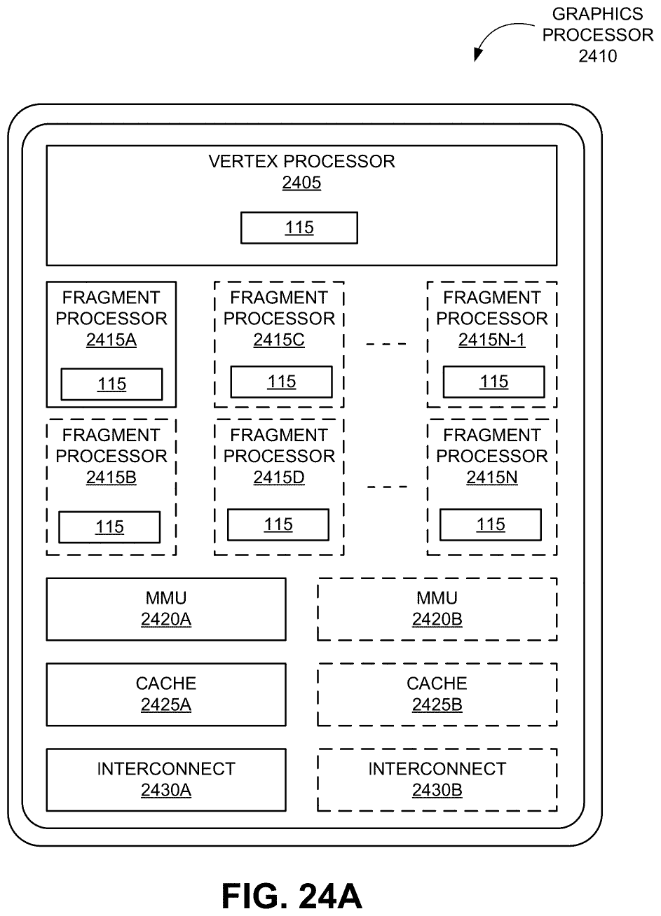

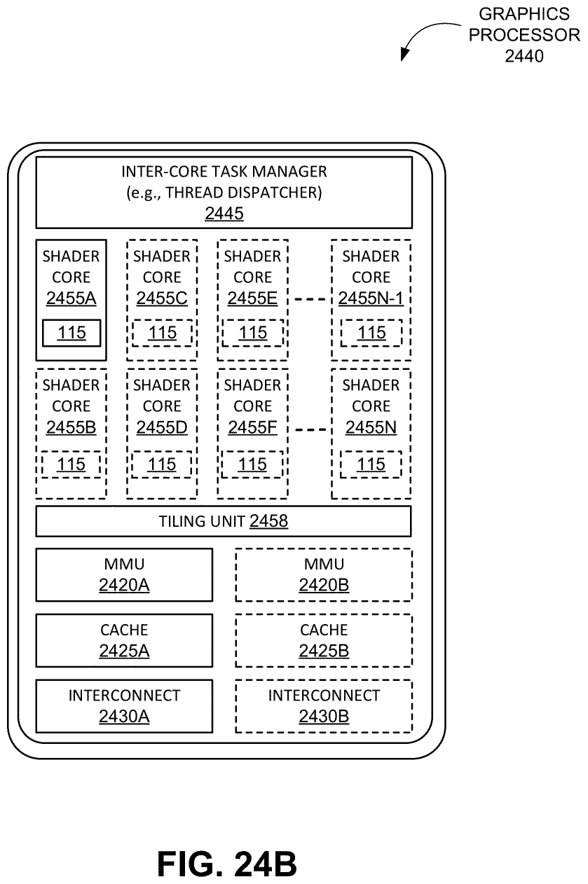

FIGS. 24A-24B illustrate exemplary integrated circuits and associated graphics processors that may be fabricated using one or more IP cores, according to various embodiments described herein. In addition to what is illustrated, other logic and circuits may be included in at least one embodiment, including additional graphics processors/cores, peripheral interface controllers, or general-purpose processor cores.

FIGS. 24A-24B are block diagrams illustrating exemplary graphics processors for use within an SoC, according to embodiments described herein. FIG. 24A illustrates an exemplary graphics processor 2410 of a system on a chip integrated circuit that may be fabricated using one or more IP cores, according to at least one embodiment. FIG. 24B illustrates an additional exemplary graphics processor 2440 of a system on a chip integrated circuit that may be fabricated using one or more IP cores, according to at least one embodiment. In at least one embodiment, graphics processor 2410 of FIG. 24A is a low power graphics processor core. In at least one embodiment, graphics processor 2440 of FIG. 24B is a higher performance graphics processor core. In at least one embodiment, each of graphics processors 2410, 2440 can be variants of graphics processor 2410 of FIG. 24.

In at least one embodiment, graphics processor 2410 includes a vertex processor 2405 and one or more fragment processor(s) 2415A-2415N (e.g., 2415A, 2415B, 2415C, 2415D, through 2415N-1, and 2415N). In at least one embodiment, graphics processor 2410 can execute different shader programs via separate logic, such that vertex processor 2405 is optimized to execute operations for vertex shader programs, while one or more fragment processor(s) 2415A-2415N execute fragment (e.g., pixel) shading operations for fragment or pixel shader programs. In at least one embodiment, vertex processor 2405 performs a vertex processing stage of a 3D graphics pipeline and generates primitives and vertex data. In at least one embodiment, fragment processor(s) 2415A-2415N use primitive and vertex data generated by vertex processor 2405 to produce a framebuffer that is displayed on a display device. In at least one embodiment, fragment processor(s) 2415A-2415N are optimized to execute fragment shader programs as provided for in an OpenGL API, which may be used to perform similar operations as a pixel shader program as provided for in a Direct 3D API.

In at least one embodiment, graphics processor 2410 additionally includes one or more memory management units (MMUs) 2420A-2420B, cache(s) 2425A-2425B, and circuit interconnect(s) 2430A-2430B. In at least one embodiment, one or more MMU(s) 2420A-2420B provide for virtual to physical address mapping for graphics processor 2410, including for vertex processor 2405 and/or fragment processor(s) 2415A-2415N, which may reference vertex or image/texture data stored in memory, in addition to vertex or image/texture data stored in one or more cache(s) 2425A-2425B. In at least one embodiment, one or more MMU(s) 2420A-2420B may be synchronized with other MMUs within a system, including one or more MMUs associated with one or more application processor(s) 2305, image processors 2315, and/or video processors 2320 of FIG. 23, such that each processor 2305-2320 can participate in a shared or unified virtual memory system. In at least one embodiment, one or more circuit interconnect(s) 2430A-2430B enable graphics processor 2410 to interface with other IP cores within SoC, either via an internal bus of SoC or via a direct connection.

In at least one embodiment, graphics processor 2440 includes one or more shader core(s) 2455A-2455N (e.g., 2455A, 2455B, 2455C, 2455D, 2455E, 2455F, through 2455N-1, and 2455N) as shown in FIG. 24B, which provides for a unified shader core architecture in which a single core or type or core can execute all types of programmable shader code, including shader program code to implement vertex shaders, fragment shaders, and/or compute shaders. In at least one embodiment, a number of shader cores can vary. In at least one embodiment, graphics processor 2440 includes an inter-core task manager 2445, which acts as a thread dispatcher to dispatch execution threads to one or more shader cores 2455A-2455N and a tiling unit 2458 to accelerate tiling operations for tile-based rendering, in which rendering operations for a scene are subdivided in image space, for example to exploit local spatial coherence within a scene or to optimize use of internal caches.

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, inference and/or training logic 115 may be used in integrated circuit 11A and/or 11B for inferencing or predicting operations based, at least in part, on weight parameters calculated using neural network training operations, neural network functions and/or architectures, or neural network use cases described herein.

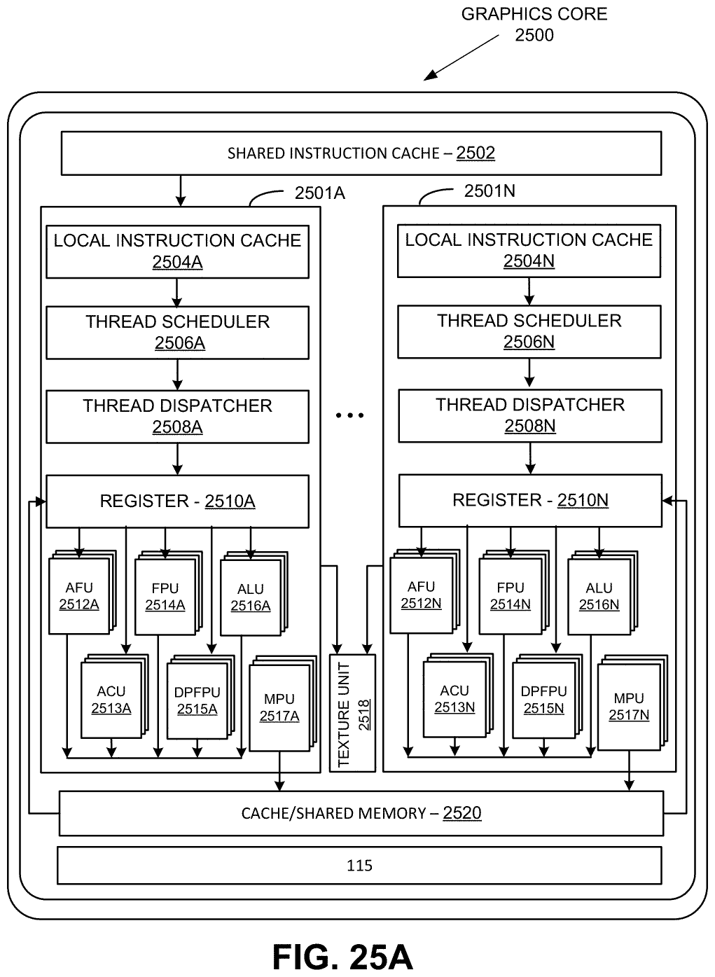

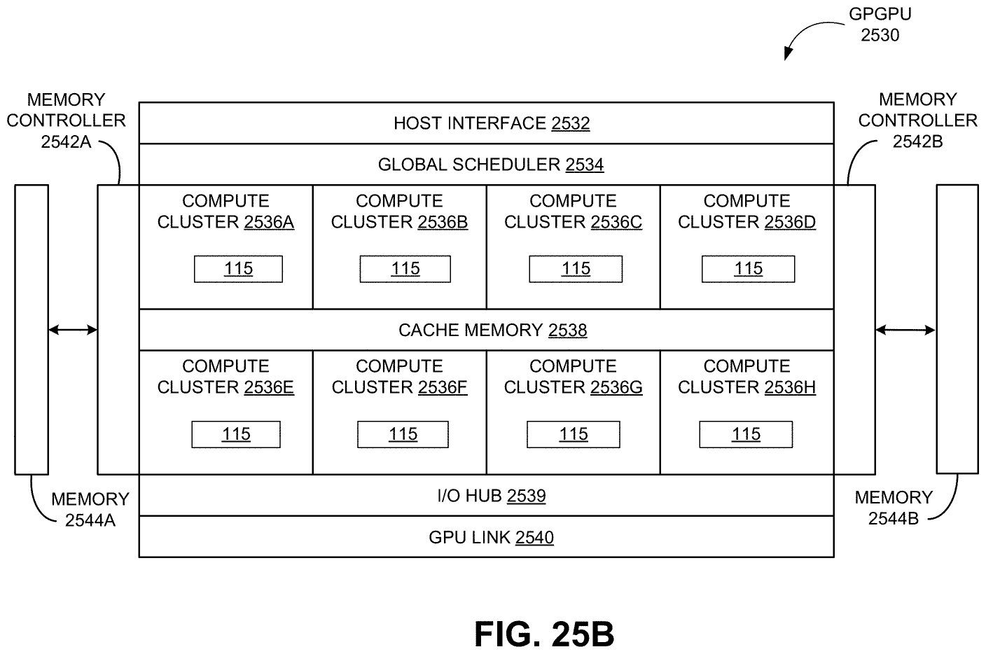

FIGS. 25A-25B illustrate additional exemplary graphics processor logic according to embodiments described herein. FIG. 25A illustrates a graphics core 2500 that may be included within graphics processor 2310 of FIG. 23, in at least one embodiment, and may be a unified shader core 2455A-2455N as in FIG. 24B in at least one embodiment. FIG. 25B illustrates a highly-parallel general-purpose graphics processing unit (“GPGPU”) 2530 suitable for deployment on a multi-chip module in at least one embodiment.

In at least one embodiment, graphics core 2500 includes a shared instruction cache 2502, a texture unit 2518, and a cache/shared memory 2520 that are common to execution resources within graphics core 2500. In at least one embodiment, graphics core 2500 can include multiple slices 2501A-2501N or a partition for each core, and a graphics processor can include multiple instances of graphics core 2500. In at least one embodiment, slices 2501A-2501N can include support logic including a local instruction cache 2504A-2504N, a thread scheduler 2506A-2506N, a thread dispatcher 2508A-2508N, and a set of registers 2510A-2510N. In at least one embodiment, slices 2501A-2501N can include a set of additional function units (AFUs 2512A-2512N), floating-point units (FPUs 2514A-2514N), integer arithmetic logic units (ALUs 2516A-2516N), address computational units (ACUs 2513A-2513N), double-precision floating-point units (DPFPUs 2515A-2515N), and matrix processing units (MPUs 2517A-2517N).

In at least one embodiment, FPUs 2514A-2514N can perform single-precision (32-bit) and half-precision (16-bit) floating point operations, while DPFPUs 2515A-2515N perform double precision (64-bit) floating point operations. In at least one embodiment, ALUs 2516A-2516N can perform variable precision integer operations at 8-bit, 16-bit, and 32-bit precision, and can be configured for mixed precision operations. In at least one embodiment, MPUs 2517A-2517N can also be configured for mixed precision matrix operations, including half-precision floating point and 8-bit integer operations. In at least one embodiment, MPUs 2517-2517N can perform a variety of matrix operations to accelerate machine learning application frameworks, including enabling support for accelerated general matrix to matrix multiplication (GEMM). In at least one embodiment, AFUs 2512A-2512N can perform additional logic operations not supported by floating-point or integer units, including trigonometric operations (e.g., sine, cosine, etc.).

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, inference and/or training logic 115 may be used in graphics core 2500 for inferencing or predicting operations based, at least in part, on weight parameters calculated using neural network training operations, neural network functions and/or architectures, or neural network use cases described herein.

FIG. 25B illustrates a general-purpose processing unit (GPGPU) 2530 that can be configured to enable highly-parallel compute operations to be performed by an array of graphics processing units, in at least one embodiment. In at least one embodiment, GPGPU 2530 can be linked directly to other instances of GPGPU 2530 to create a multi-GPU cluster to improve training speed for deep neural networks. In at least one embodiment, GPGPU 2530 includes a host interface 2532 to enable a connection with a host processor. In at least one embodiment, host interface 2532 is a PCI Express interface. In at least one embodiment, host interface 2532 can be a vendor-specific communications interface or communications fabric. In at least one embodiment, GPGPU 2530 receives commands from a host processor and uses a global scheduler 2534 to distribute execution threads associated with those commands to a set of compute clusters 2536A-2536H. In at least one embodiment, compute clusters 2536A-2536H share a cache memory 2538. In at least one embodiment, cache memory 2538 can serve as a higher-level cache for cache memories within compute clusters 2536A-2536H.

In at least one embodiment, GPGPU 2530 includes memory 2544A-2544B coupled with compute clusters 2536A-2536H via a set of memory controllers 2542A-2542B. In at least one embodiment, memory 2544A-2544B can include various types of memory devices including dynamic random access memory (DRAM) or graphics random access memory, such as synchronous graphics random access memory (SGRAM), including graphics double data rate (GDDR) memory.

In at least one embodiment, compute clusters 2536A-2536H each include a set of graphics cores, such as graphics core 2500 of FIG. 25A, which can include multiple types of integer and floating point logic units that can perform computational operations at a range of precisions including suited for machine learning computations. For example, in at least one embodiment, at least a subset of floating point units in each of compute clusters 2536A-2536H can be configured to perform 16-bit or 32-bit floating point operations, while a different subset of floating point units can be configured to perform 64-bit floating point operations.

In at least one embodiment, multiple instances of GPGPU 2530 can be configured to operate as a compute cluster. In at least one embodiment, communication used by compute clusters 2536A-2536H for synchronization and data exchange varies across embodiments. In at least one embodiment, multiple instances of GPGPU 2530 communicate over host interface 2532. In at least one embodiment, GPGPU 2530 includes an I/O hub 2539 that couples GPGPU 2530 with a GPU link 2540 that enables a direct connection to other instances of GPGPU 2530. In at least one embodiment, GPU link 2540 is coupled to a dedicated GPU-to-GPU bridge that enables communication and synchronization between multiple instances of GPGPU 2530. In at least one embodiment, GPU link 2540 couples with a high-speed interconnect to transmit and receive data to other GPGPUs or parallel processors. In at least one embodiment, multiple instances of GPGPU 2530 are located in separate data processing systems and communicate via a network device that is accessible via host interface 2532. In at least one embodiment GPU link 2540 can be configured to enable a connection to a host processor in addition to or as an alternative to host interface 2532.

In at least one embodiment, GPGPU 2530 can be configured to train neural networks. In at least one embodiment, GPGPU 2530 can be used within an inferencing platform. In at least one embodiment, in which GPGPU 2530 is used for inferencing, GPGPU 2530 may include fewer compute clusters 2536A-2536H relative to when GPGPU 2530 is used for training a neural network. In at least one embodiment, memory technology associated with memory 2544A-2544B may differ between inferencing and training configurations, with higher bandwidth memory technologies devoted to training configurations. In at least one embodiment, an inferencing configuration of GPGPU 2530 can support inferencing specific instructions. For example, in at least one embodiment, an inferencing configuration can provide support for one or more 8-bit integer dot product instructions, which may be used during inferencing operations for deployed neural networks.

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, inference and/or training logic 115 may be used in GPGPU 2530 for inferencing or predicting operations based, at least in part, on weight parameters calculated using neural network training operations, neural network functions and/or architectures, or neural network use cases described herein.

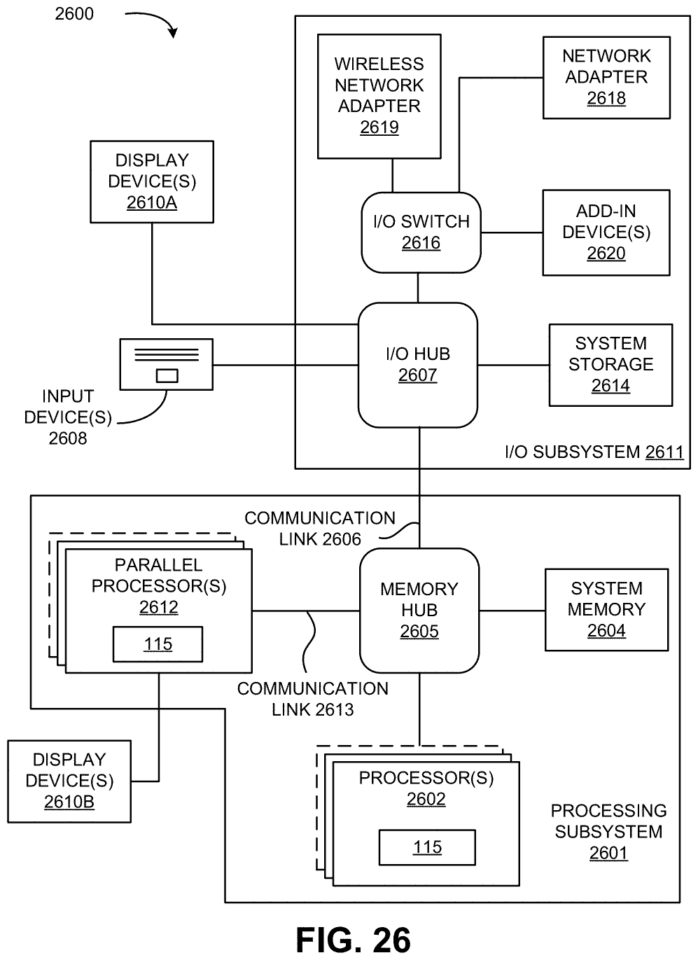

FIG. 26 is a block diagram illustrating a computing system 2600 according to at least one embodiment. In at least one embodiment, computing system 2600 includes a processing subsystem 2601 having one or more processor(s) 2602 and a system memory 2604 communicating via an interconnection path that may include a memory hub 2605. In at least one embodiment, memory hub 2605 may be a separate component within a chipset component or may be integrated within one or more processor(s) 2602. In at least one embodiment, memory hub 2605 couples with an I/O subsystem 2611 via a communication link 2606. In at least one embodiment, I/O subsystem 2611 includes an I/O hub 2607 that can enable computing system 2600 to receive input from one or more input device(s) 2608. In at least one embodiment, I/O hub 2607 can enable a display controller, which may be included in one or more processor(s) 2602, to provide outputs to one or more display device(s) 2610A. In at least one embodiment, one or more display device(s) 2610A coupled with I/O hub 2607 can include a local, internal, or embedded display device.

In at least one embodiment, processing subsystem 2601 includes one or more parallel processor(s) 2612 coupled to memory hub 2605 via a bus or other communication link 2613. In at least one embodiment, communication link 2613 may use one of any number of standards based communication link technologies or protocols, such as, but not limited to PCI Express, or may be a vendor-specific communications interface or communications fabric. In at least one embodiment, one or more parallel processor(s) 2612 form a computationally focused parallel or vector processing system that can include a large number of processing cores and/or processing clusters, such as a many-integrated core (MIC) processor. In at least one embodiment, some or all of parallel processor(s) 2612 form a graphics processing subsystem that can output pixels to one of one or more display device(s) 2610A coupled via I/O Hub 2607. In at least one embodiment, parallel processor(s) 2612 can also include a display controller and display interface (not shown) to enable a direct connection to one or more display device(s) 2610B.

In at least one embodiment, a system storage unit 2614 can connect to I/O hub 2607 to provide a storage mechanism for computing system 2600. In at least one embodiment, an I/O switch 2616 can be used to provide an interface mechanism to enable connections between I/O hub 2607 and other components, such as a network adapter 2618 and/or a wireless network adapter 2619 that may be integrated into platform, and various other devices that can be added via one or more add-in device(s) 2620. In at least one embodiment, network adapter 2618 can be an Ethernet adapter or another wired network adapter. In at least one embodiment, wireless network adapter 2619 can include one or more of a Wi-Fi, Bluetooth, near field communication (NFC), or other network device that includes one or more wireless radios.

In at least one embodiment, computing system 2600 can include other components not explicitly shown, including USB or other port connections, optical storage drives, video capture devices, and like, may also be connected to I/O hub 2607. In at least one embodiment, communication paths interconnecting various components in FIG. 26 may be implemented using any suitable protocols, such as PCI (Peripheral Component Interconnect) based protocols (e.g., PCI-Express), or other bus or point-to-point communication interfaces and/or protocol(s), such as NV-Link high-speed interconnect, or interconnect protocols.

In at least one embodiment, parallel processor(s) 2612 incorporate circuitry optimized for graphics and video processing, including, for example, video output circuitry, and constitutes a graphics processing unit (GPU). In at least one embodiment, parallel processor(s) 2612 incorporate circuitry optimized for general purpose processing. In at least embodiment, components of computing system 2600 may be integrated with one or more other system elements on a single integrated circuit. For example, in at least one embodiment, parallel processor(s) 2612, memory hub 2605, processor(s) 2602, and I/O hub 2607 can be integrated into a system on chip (SoC) integrated circuit. In at least one embodiment, components of computing system 2600 can be integrated into a single package to form a system in package (SIP) configuration. In at least one embodiment, at least a portion of components of computing system 2600 can be integrated into a multi-chip module (MCM), which can be interconnected with other multi-chip modules into a modular computing system.

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, inference and/or training logic 115 may be used computing system 2600 of FIG. 26 for inferencing or predicting operations based, at least in part, on weight parameters calculated using neural network training operations, neural network functions and/or architectures, or neural network use cases described herein.

### Processors

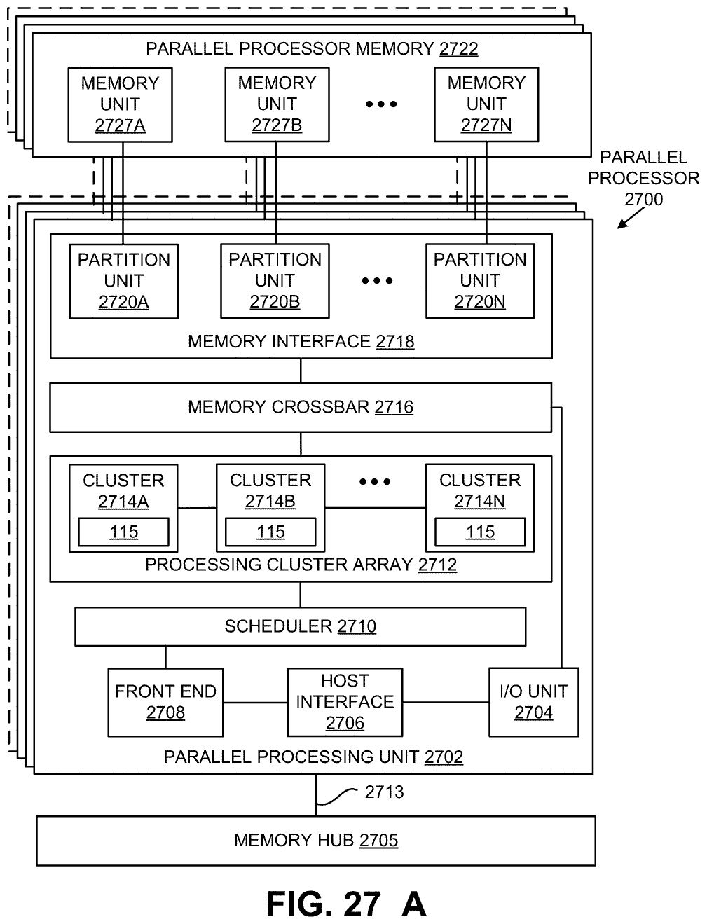

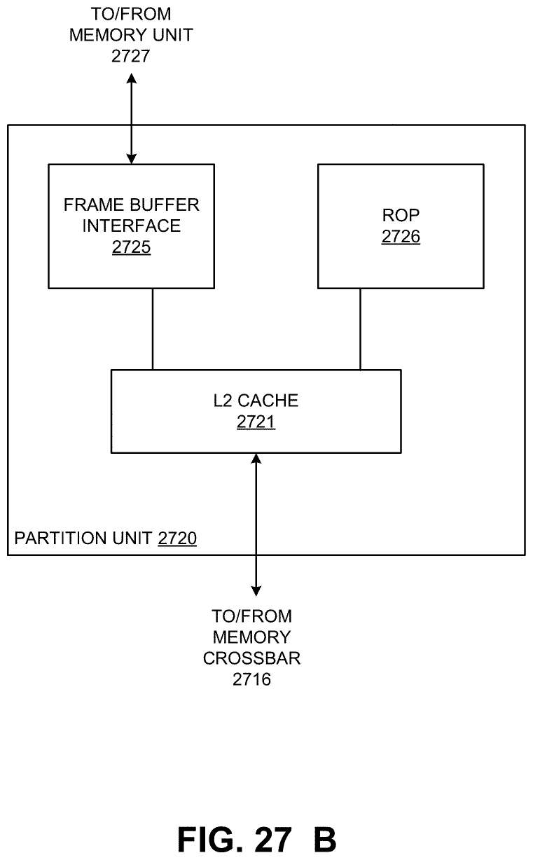

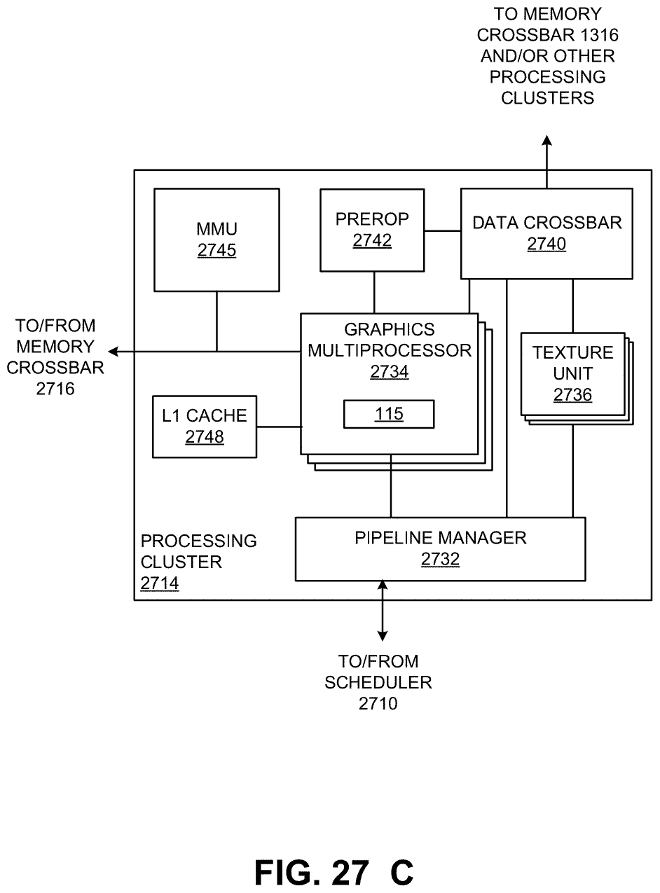

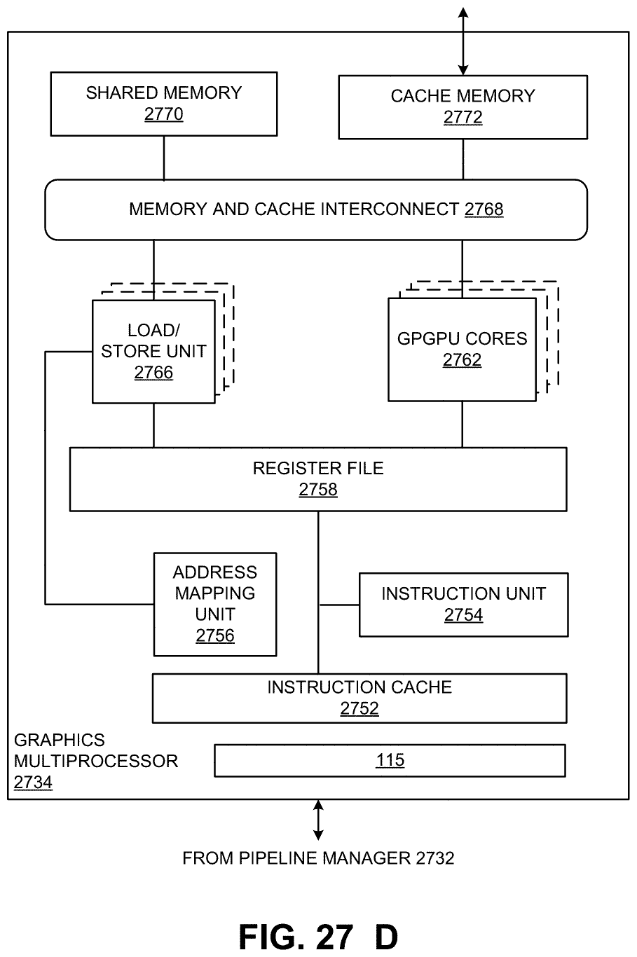

FIG. 27A illustrates a parallel processor 2700 according to at least one embodiment. In at least one embodiment, various components of parallel processor 2700 may be implemented using one or more integrated circuit devices, such as programmable processors, application specific integrated circuits (ASICs), or field programmable gate arrays (FPGA). In at least one embodiment, illustrated parallel processor 2700 is a variant of one or more parallel processor(s) 2612 shown in FIG. 26 according to an exemplary embodiment.

In at least one embodiment, parallel processor 2700 includes a parallel processing unit 2702. In at least one embodiment, parallel processing unit 2702 includes an I/O unit 2704 that enables communication with other devices, including other instances of parallel processing unit 2702. In at least one embodiment, I/O unit 2704 may be directly connected to other devices. In at least one embodiment, I/O unit 2704 connects with other devices via use of a hub or switch interface, such as a memory hub 2705. In at least one embodiment, connections between memory hub 2705 and I/O unit 2704 form a communication link 2713. In at least one embodiment, I/O unit 2704 connects with a host interface 2706 and a memory crossbar 2716, where host interface 2706 receives commands directed to performing processing operations and memory crossbar 2716 receives commands directed to performing memory operations.

In at least one embodiment, when host interface 2706 receives a command buffer via I/O unit 2704, host interface 2706 can direct work operations to perform those commands to a front end 2708. In at least one embodiment, front end 2708 couples with a scheduler 2710, which is configured to distribute commands or other work items to a processing cluster array 2712. In at least one embodiment, scheduler 2710 ensures that processing cluster array 2712 is properly configured and in a valid state before tasks are distributed to a cluster of processing cluster array 2712. In at least one embodiment, scheduler 2710 is implemented via firmware logic executing on a microcontroller. In at least one embodiment, microcontroller implemented scheduler 2710 is configurable to perform complex scheduling and work distribution operations at coarse and fine granularity, enabling rapid preemption and context switching of threads executing on processing array 2712. In at least one embodiment, host software can prove workloads for scheduling on processing cluster array 2712 via one of multiple graphics processing paths. In at least one embodiment, workloads can then be automatically distributed across processing array cluster 2712 by scheduler 2710 logic within a microcontroller including scheduler 2710.

In at least one embodiment, processing cluster array 2712 can include up to “N” processing clusters (e.g., cluster 2714A, cluster 2714B, through cluster 2714N), where “N” represents a positive integer (which may be a different integer “N” than used in other figures). In at least one embodiment, each cluster 2714A-2714N of processing cluster array 2712 can execute a large number of concurrent threads. In at least one embodiment, scheduler 2710 can allocate work to clusters 2714A-2714N of processing cluster array 2712 using various scheduling and/or work distribution algorithms, which may vary depending on workload arising for each type of program or computation. In at least one embodiment, scheduling can be handled dynamically by scheduler 2710, or can be assisted in part by compiler logic during compilation of program logic configured for execution by processing cluster array 2712. In at least one embodiment, different clusters 2714A-2714N of processing cluster array 2712 can be allocated for processing different types of programs or for performing different types of computations.

In at least one embodiment, processing cluster array 2712 can be configured to perform various types of parallel processing operations. In at least one embodiment, processing cluster array 2712 is configured to perform general-purpose parallel compute operations. For example, in at least one embodiment, processing cluster array 2712 can include logic to execute processing tasks including filtering of video and/or audio data, performing modeling operations, including physics operations, and performing data transformations.

In at least one embodiment, processing cluster array 2712 is configured to perform parallel graphics processing operations. In at least one embodiment, processing cluster array 2712 can include additional logic to support execution of such graphics processing operations, including but not limited to, texture sampling logic to perform texture operations, as well as tessellation logic and other vertex processing logic. In at least one embodiment, processing cluster array 2712 can be configured to execute graphics processing related shader programs such as, but not limited to, vertex shaders, tessellation shaders, geometry shaders, and pixel shaders. In at least one embodiment, parallel processing unit 2702 can transfer data from system memory via I/O unit 2704 for processing. In at least one embodiment, during processing, transferred data can be stored to on-chip memory (e.g., parallel processor memory 2722) during processing, then written back to system memory.

In at least one embodiment, when parallel processing unit 2702 is used to perform graphics processing, scheduler 2710 can be configured to divide a processing workload into approximately equal sized tasks, to better enable distribution of graphics processing operations to multiple clusters 2714A-2714N of processing cluster array 2712. In at least one embodiment, portions of processing cluster array 2712 can be configured to perform different types of processing. For example, in at least one embodiment, a first portion may be configured to perform vertex shading and topology generation, a second portion may be configured to perform tessellation and geometry shading, and a third portion may be configured to perform pixel shading or other screen space operations, to produce a rendered image for display. In at least one embodiment, intermediate data produced by one or more of clusters 2714A-2714N may be stored in buffers to allow intermediate data to be transmitted between clusters 2714A-2714N for further processing.

In at least one embodiment, processing cluster array 2712 can receive processing tasks to be executed via scheduler 2710, which receives commands defining processing tasks from front end 2708. In at least one embodiment, processing tasks can include indices of data to be processed, e.g., surface (patch) data, primitive data, vertex data, and/or pixel data, as well as state parameters and commands defining how data is to be processed (e.g., what program is to be executed). In at least one embodiment, scheduler 2710 may be configured to fetch indices corresponding to tasks or may receive indices from front end 2708. In at least one embodiment, front end 2708 can be configured to ensure processing cluster array 2712 is configured to a valid state before a workload specified by incoming command buffers (e.g., batch-buffers, push buffers, etc.) is initiated.

In at least one embodiment, each of one or more instances of parallel processing unit 2702 can couple with a parallel processor memory 2722. In at least one embodiment, parallel processor memory 2722 can be accessed via memory crossbar 2716, which can receive memory requests from processing cluster array 2712 as well as I/O unit 2704. In at least one embodiment, memory crossbar 2716 can access parallel processor memory 2722 via a memory interface 2718. In at least one embodiment, memory interface 2718 can include multiple partition units (e.g., partition unit 2720A, partition unit 2720B, through partition unit 2720N) that can each couple to a portion (e.g., memory unit) of parallel processor memory 2722. In at least one embodiment, a number of partition units 2720A-2720N is configured to be equal to a number of memory units, such that a first partition unit 2720A has a corresponding first memory unit 2724A, a second partition unit 2720B has a corresponding memory unit 2724B, and an N-th partition unit 2720N has a corresponding N-th memory unit 2724N. In at least one embodiment, a number of partition units 2720A-2720N may not be equal to a number of memory units.

In at least one embodiment, memory units 2724A-2724N can include various types of memory devices, including dynamic random access memory (DRAM) or graphics random access memory, such as synchronous graphics random access memory (SGRAM), including graphics double data rate (GDDR) memory. In at least one embodiment, memory units 2724A-2724N may also include 3D stacked memory, including but not limited to high bandwidth memory (HBM). In at least one embodiment, render targets, such as frame buffers or texture maps may be stored across memory units 2724A-2724N, allowing partition units 2720A-2720N to write portions of each render target in parallel to efficiently use available bandwidth of parallel processor memory 2722. In at least one embodiment, a local instance of parallel processor memory 2722 may be excluded in favor of a unified memory design that utilizes system memory in conjunction with local cache memory.

In at least one embodiment, any one of clusters 2714A-2714N of processing cluster array 2712 can process data that will be written to any of memory units 2724A-2724N within parallel processor memory 2722. In at least one embodiment, memory crossbar 2716 can be configured to transfer an output of each cluster 2714A-2714N to any partition unit 2720A-2720N or to another cluster 2714A-2714N, which can perform additional processing operations on an output. In at least one embodiment, each cluster 2714A-2714N can communicate with memory interface 2718 through memory crossbar 2716 to read from or write to various external memory devices. In at least one embodiment, memory crossbar 2716 has a connection to memory interface 2718 to communicate with I/O unit 2704, as well as a connection to a local instance of parallel processor memory 2722, enabling processing units within different processing clusters 2714A-2714N to communicate with system memory or other memory that is not local to parallel processing unit 2702. In at least one embodiment, memory crossbar 2716 can use virtual channels to separate traffic streams between clusters 2714A-2714N and partition units 2720A-2720N.

In at least one embodiment, multiple instances of parallel processing unit 2002 can be provided on a single add-in card, or multiple add-in cards can be interconnected. In at least one embodiment, different instances of parallel processing unit 2702 can be configured to interoperate even if different instances have different numbers of processing cores, different amounts of local parallel processor memory, and/or other configuration differences. For example, in at least one embodiment, some instances of parallel processing unit 2702 can include higher precision floating point units relative to other instances. In at least one embodiment, systems incorporating one or more instances of parallel processing unit 2702 or parallel processor 2700 can be implemented in a variety of configurations and form factors, including but not limited to desktop, laptop, or handheld personal computers, servers, workstations, game consoles, and/or embedded systems.

FIG. 27B is a block diagram of a partition unit 2720 according to at least one embodiment. In at least one embodiment, partition unit 2720 is an instance of one of partition units 2720A-2720N of FIG. 27A. In at least one embodiment, partition unit 2720 includes an L2 cache 2021, a frame buffer interface 2725, and a ROP 2727 (raster operations unit). In at least one embodiment, L2 cache 2721 is a read/write cache that is configured to perform load and store operations received from memory crossbar 2716 and ROP 2727. In at least one embodiment, read misses and urgent write-back requests are output by L2 cache 2721 to frame buffer interface 2725 for processing. In at least one embodiment, updates can also be sent to a frame buffer via frame buffer interface 2725 for processing. In at least one embodiment, frame buffer interface 2725 interfaces with one of memory units in parallel processor memory, such as memory units 2724A-2724N of FIG. 27 (e.g., within parallel processor memory 2722).

In at least one embodiment, ROP 2726 is a processing unit that performs raster operations such as stencil, z test, blending, etc. In at least one embodiment, ROP 2726 then outputs processed graphics data that is stored in graphics memory. In at least one embodiment, ROP 2726 includes compression logic to compress depth or color data that is written to memory and decompress depth or color data that is read from memory. In at least one embodiment, compression logic can be lossless compression logic that makes use of one or more of multiple compression algorithms. In at least one embodiment, a type of compression that is performed by ROP 2726 can vary based on statistical characteristics of data to be compressed. For example, in at least one embodiment, delta color compression is performed on depth and color data on a per-tile basis.

In at least one embodiment, ROP 2726 is included within each processing cluster (e.g., cluster 2714A-2714N of FIG. 27A) instead of within partition unit 2720. In at least one embodiment, read and write requests for pixel data are transmitted over memory crossbar 2716 instead of pixel fragment data. In at least one embodiment, processed graphics data may be displayed on a display device, such as one of one or more display device(s) 2510 of FIG. 25, routed for further processing by processor(s) 1302, or routed for further processing by one of processing entities within parallel processor 2700 of FIG. 27A.

FIG. 27C is a block diagram of a processing cluster 2714 within a parallel processing unit according to at least one embodiment. In at least one embodiment, a processing cluster is an instance of one of processing clusters 2714A-2714N of FIG. 27A. In at least one embodiment, processing cluster 2714 can be configured to execute many threads in parallel, where “thread” refers to an instance of a particular program executing on a particular set of input data. In at least one embodiment, single-instruction, multiple-data (SIMD) instruction issue techniques are used to support parallel execution of a large number of threads without providing multiple independent instruction units. In at least one embodiment, single-instruction, multiple-thread (SIMT) techniques are used to support parallel execution of a large number of generally synchronized threads, using a common instruction unit configured to issue instructions to a set of processing engines within each one of processing clusters.

In at least one embodiment, operation of processing cluster 2714 can be controlled via a pipeline manager 2732 that distributes processing tasks to SIMT parallel processors. In at least one embodiment, pipeline manager 2732 receives instructions from scheduler 2710 of FIG. 27A and manages execution of those instructions via a graphics multiprocessor 2734 and/or a texture unit 2736. In at least one embodiment, graphics multiprocessor 2734 is an exemplary instance of a SIMT parallel processor. However, in at least one embodiment, various types of SIMT parallel processors of differing architectures may be included within processing cluster 2714. In at least one embodiment, one or more instances of graphics multiprocessor 2734 can be included within a processing cluster 2714. In at least one embodiment, graphics multiprocessor 2734 can process data and a data crossbar 2740 can be used to distribute processed data to one of multiple possible destinations, including other shader units. In at least one embodiment, pipeline manager 2732 can facilitate distribution of processed data by specifying destinations for processed data to be distributed via data crossbar 2740.

In at least one embodiment, each graphics multiprocessor 2734 within processing cluster 2714 can include an identical set of functional execution logic (e.g., arithmetic logic units, load-store units, etc.). In at least one embodiment, functional execution logic can be configured in a pipelined manner in which new instructions can be issued before previous instructions are complete. In at least one embodiment, functional execution logic supports a variety of operations including integer and floating point arithmetic, comparison operations, Boolean operations, bit-shifting, and computation of various algebraic functions. In at least one embodiment, same functional-unit hardware can be leveraged to perform different operations and any combination of functional units may be present.

In at least one embodiment, instructions transmitted to processing cluster 2714 constitute a thread. In at least one embodiment, a set of threads executing across a set of parallel processing engines is a thread group. In at least one embodiment, a thread group executes a common program on different input data. In at least one embodiment, each thread within a thread group can be assigned to a different processing engine within a graphics multiprocessor 2734. In at least one embodiment, a thread group may include fewer threads than a number of processing engines within graphics multiprocessor 2734. In at least one embodiment, when a thread group includes fewer threads than a number of processing engines, one or more of processing engines may be idle during cycles in which that thread group is being processed. In at least one embodiment, a thread group may also include more threads than a number of processing engines within graphics multiprocessor 2734. In at least one embodiment, when a thread group includes more threads than number of processing engines within graphics multiprocessor 2734, processing can be performed over consecutive clock cycles. In at least one embodiment, multiple thread groups can be executed concurrently on a graphics multiprocessor 2734.

In at least one embodiment, graphics multiprocessor 2734 includes an internal cache memory to perform load and store operations. In at least one embodiment, graphics multiprocessor 2734 can forego an internal cache and use a cache memory (e.g., L1 cache 2748) within processing cluster 2714. In at least one embodiment, each graphics multiprocessor 2734 also has access to L2 caches within partition units (e.g., partition units 2720A-2720N of FIG. 27A) that are shared among all processing clusters 2714 and may be used to transfer data between threads. In at least one embodiment, graphics multiprocessor 2734 may also access off-chip global memory, which can include one or more of local parallel processor memory and/or system memory. In at least one embodiment, any memory external to parallel processing unit 2702 may be used as global memory. In at least one embodiment, processing cluster 2714 includes multiple instances of graphics multiprocessor 2734 and can share common instructions and data, which may be stored in L1 cache 2748.

In at least one embodiment, each processing cluster 2714 may include an MMU 2745 (memory management unit) that is configured to map virtual addresses into physical addresses. In at least one embodiment, one or more instances of MMU 2745 may reside within memory interface 2718 of FIG. 27A. In at least one embodiment, MMU 2745 includes a set of page table entries (PTEs) used to map a virtual address to a physical address of a tile and optionally a cache line index. In at least one embodiment, MMU 2745 may include address translation lookaside buffers (TLB) or caches that may reside within graphics multiprocessor 2734 or L1 2748 cache or processing cluster 2714. In at least one embodiment, a physical address is processed to distribute surface data access locally to allow for efficient request interleaving among partition units. In at least one embodiment, a cache line index may be used to determine whether a request for a cache line is a hit or miss.

In at least one embodiment, a processing cluster 2714 may be configured such that each graphics multiprocessor 2734 is coupled to a texture unit 2736 for performing texture mapping operations, e.g., determining texture sample positions, reading texture data, and filtering texture data. In at least one embodiment, texture data is read from an internal texture L1 cache (not shown) or from an L1 cache within graphics multiprocessor 2734 and is fetched from an L2 cache, local parallel processor memory, or system memory, as needed. In at least one embodiment, each graphics multiprocessor 2734 outputs processed tasks to data crossbar 2740 to provide processed task to another processing cluster 2714 for further processing or to store processed task in an L2 cache, local parallel processor memory, or system memory via memory crossbar 2716. In at least one embodiment, a preROP 2742 (pre-raster operations unit) is configured to receive data from graphics multiprocessor 2734, and direct data to ROP units, which may be located with partition units as described herein (e.g., partition units 2720A-2720N of FIG. 27A). In at least one embodiment, preROP 2742 unit can perform optimizations for color blending, organizing pixel color data, and performing address translations.

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, inference and/or training logic 115 may be used in graphics processing cluster 2714 for inferencing or predicting operations based, at least in part, on weight parameters calculated using neural network training operations, neural network functions and/or architectures, or neural network use cases described herein.

FIG. 27D shows a graphics multiprocessor 2734 according to at least one embodiment. In at least one embodiment, graphics multiprocessor 2734 couples with pipeline manager 2732 of processing cluster 2714. In at least one embodiment, graphics multiprocessor 2734 has an execution pipeline including but not limited to an instruction cache 2752, an instruction unit 2754, an address mapping unit 2756, a register file 2758, one or more general purpose graphics processing unit (GPGPU) cores 2762, and one or more load/store units 2766. In at least one embodiment, GPGPU cores 2762 and load/store units 2766 are coupled with cache memory 2772 and shared memory 2770 via a memory and cache interconnect 2768.

In at least one embodiment, instruction cache 2752 receives a stream of instructions to execute from pipeline manager 2732. In at least one embodiment, instructions are cached in instruction cache 2752 and dispatched for execution by an instruction unit 2754. In at least one embodiment, instruction unit 2754 can dispatch instructions as thread groups (e.g., warps), with each thread of thread group assigned to a different execution unit within GPGPU cores 2762. In at least one embodiment, an instruction can access any of a local, shared, or global address space by specifying an address within a unified address space. In at least one embodiment, address mapping unit 2756 can be used to translate addresses in a unified address space into a distinct memory address that can be accessed by load/store units 2766.

In at least one embodiment, register file 2758 provides a set of registers for functional units of graphics multiprocessor 2734. In at least one embodiment, register file 2758 provides temporary storage for operands connected to data paths of functional units (e.g., GPGPU cores 2762, load/store units 2766) of graphics multiprocessor 2734. In at least one embodiment, register file 2758 is divided between each of functional units such that each functional unit is allocated a dedicated portion of register file 2758. In at least one embodiment, register file 2758 is divided between different warps being executed by graphics multiprocessor 2734.

In at least one embodiment, GPGPU cores 2762 can each include floating point units (FPUs) and/or integer arithmetic logic units (ALUs) that are used to execute instructions of graphics multiprocessor 2734. In at least one embodiment, GPGPU cores 2762 can be similar in architecture or can differ in architecture. In at least one embodiment, a first portion of GPGPU cores 2762 include a single precision FPU and an integer ALU while a second portion of GPGPU cores include a double precision FPU. In at least one embodiment, FPUs can implement IEEE 754-2008 standard floating point arithmetic or enable variable precision floating point arithmetic. In at least one embodiment, graphics multiprocessor 2734 can additionally include one or more fixed function or special function units to perform specific functions such as copy rectangle or pixel blending operations. In at least one embodiment, one or more of GPGPU cores 2762 can also include fixed or special function logic.

In at least one embodiment, GPGPU cores 2762 include SIMD logic capable of performing a single instruction on multiple sets of data. In at least one embodiment, GPGPU cores 2762 can physically execute SIMD4, SIMD8, and SIMD16 instructions and logically execute SIMD1, SIMD2, and SIMD32 instructions. In at least one embodiment, SIMD instructions for GPGPU cores can be generated at compile time by a shader compiler or automatically generated when executing programs written and compiled for single program multiple data (SPMD) or SIMT architectures. In at least one embodiment, multiple threads of a program configured for an SIMT execution model can executed via a single SIMD instruction. For example, in at least one embodiment, eight SIMT threads that perform same or similar operations can be executed in parallel via a single SIMD8 logic unit.

In at least one embodiment, memory and cache interconnect 2768 is an interconnect network that connects each functional unit of graphics multiprocessor 2734 to register file 2758 and to shared memory 2770. In at least one embodiment, memory and cache interconnect 2768 is a crossbar interconnect that allows load/store unit 2766 to implement load and store operations between shared memory 2770 and register file 2758. In at least one embodiment, register file 2758 can operate at a same frequency as GPGPU cores 2762, thus data transfer between GPGPU cores 2762 and register file 2758 can have very low latency. In at least one embodiment, shared memory 2770 can be used to enable communication between threads that execute on functional units within graphics multiprocessor 2734. In at least one embodiment, cache memory 2772 can be used as a data cache for example, to cache texture data communicated between functional units and texture unit 2736. In at least one embodiment, shared memory 2770 can also be used as a program managed cache. In at least one embodiment, threads executing on GPGPU cores 2762 can programmatically store data within shared memory in addition to automatically cached data that is stored within cache memory 2772.

In at least one embodiment, a parallel processor or GPGPU as described herein is communicatively coupled to host/processor cores to accelerate graphics operations, machine-learning operations, pattern analysis operations, and various general purpose GPU (GPGPU) functions. In at least one embodiment, a GPU may be communicatively coupled to host processor/cores over a bus or other interconnect (e.g., a high-speed interconnect such as PCIe or NVLink). In at least one embodiment, a GPU may be integrated on a package or chip as cores and communicatively coupled to cores over an internal processor bus/interconnect internal to a package or chip. In at least one embodiment, regardless a manner in which a GPU is connected, processor cores may allocate work to such GPU in a form of sequences of commands/instructions contained in a work descriptor. In at least one embodiment, that GPU then uses dedicated circuitry/logic for efficiently processing these commands/instructions.

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, inference and/or training logic 115 may be used in graphics multiprocessor 2734 for inferencing or predicting operations based, at least in part, on weight parameters calculated using neural network training operations, neural network functions and/or architectures, or neural network use cases described herein.

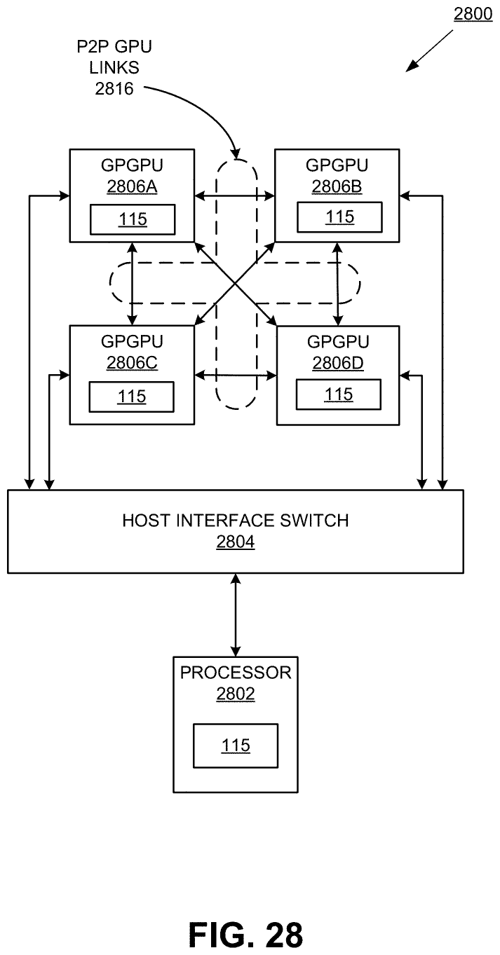

FIG. 28 illustrates a multi-GPU computing system 2800, according to at least one embodiment. In at least one embodiment, multi-GPU computing system 2800 can include a processor 2802 coupled to multiple general purpose graphics processing units (GPGPUs) 2806A-D via a host interface switch 2804. In at least one embodiment, host interface switch 2804 is a PCI express switch device that couples processor 2802 to a PCI express bus over which processor 2802 can communicate with GPGPUs 2806A-D. In at least one embodiment, GPGPUs 2806A-D can interconnect via a set of high-speed point-to-point GPU-to-GPU links 2816. In at least one embodiment, GPU-to-GPU links 2816 connect to each of GPGPUs 2806A-D via a dedicated GPU link. In at least one embodiment, P2P GPU links 2816 enable direct communication between each of GPGPUs 2806A-D without requiring communication over host interface bus 2804 to which processor 2802 is connected. In at least one embodiment, with GPU-to-GPU traffic directed to P2P GPU links 2816, host interface bus 2804 remains available for system memory access or to communicate with other instances of multi-GPU computing system 2800, for example, via one or more network devices. While in at least one embodiment GPGPUs 2806A-D connect to processor 2802 via host interface switch 2804, in at least one embodiment processor 2802 includes direct support for P2P GPU links 2816 and can connect directly to GPGPUs 2806A-D.

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, inference and/or training logic 115 may be used in multi-GPU computing system 2800 for inferencing or predicting operations based, at least in part, on weight parameters calculated using neural network training operations, neural network functions and/or architectures, or neural network use cases described herein.

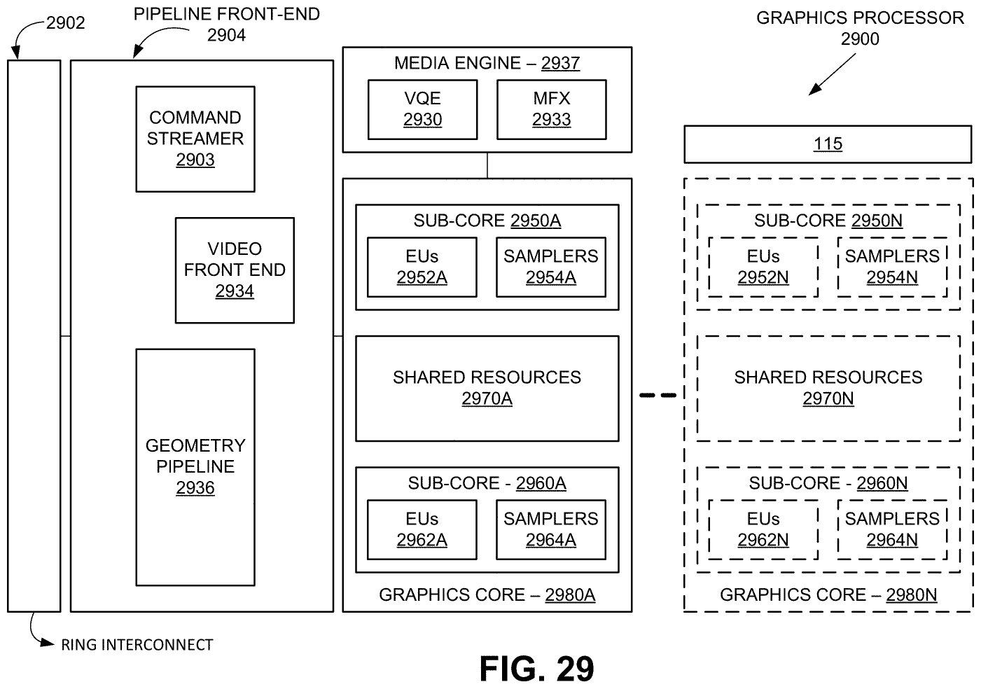

FIG. 29 is a block diagram of a graphics processor 2900, according to at least one embodiment. In at least one embodiment, graphics processor 2900 includes a ring interconnect 2902, a pipeline front-end 2904, a media engine 2937, and graphics cores 2980A-2980N. In at least one embodiment, ring interconnect 2902 couples graphics processor 2900 to other processing units, including other graphics processors or one or more general-purpose processor cores. In at least one embodiment, graphics processor 2900 is one of many processors integrated within a multi-core processing system.

In at least one embodiment, graphics processor 2900 receives batches of commands via ring interconnect 2902. In at least one embodiment, incoming commands are interpreted by a command streamer 2903 in pipeline front-end 2904. In at least one embodiment, graphics processor 2900 includes scalable execution logic to perform 3D geometry processing and media processing via graphics core(s) 2980A-2980N. In at least one embodiment, for 3D geometry processing commands, command streamer 2903 supplies commands to geometry pipeline 2936. In at least one embodiment, for at least some media processing commands, command streamer 2903 supplies commands to a video front end 2934, which couples with media engine 2937. In at least one embodiment, media engine 2937 includes a Video Quality Engine (VQE) 2930 for video and image post-processing and a multi-format encode/decode (MFX) 2933 engine to provide hardware-accelerated media data encoding and decoding. In at least one embodiment, geometry pipeline 2936 and media engine 2937 each generate execution threads for thread execution resources provided by at least one graphics core 2980.

In at least one embodiment, graphics processor 2900 includes scalable thread execution resources featuring graphics cores 2980A-2980N (which can be modular and are sometimes referred to as core slices), each having multiple sub-cores 2950A-50N, 2960A-2960N (sometimes referred to as core sub-slices). In at least one embodiment, graphics processor 2900 can have any number of graphics cores 2980A. In at least one embodiment, graphics processor 2900 includes a graphics core 2980A having at least a first sub-core 2950A and a second sub-core 2960A. In at least one embodiment, graphics processor 2900 is a low power processor with a single sub-core (e.g., 2950A). In at least one embodiment, graphics processor 2900 includes multiple graphics cores 2980A-2980N, each including a set of first sub-cores 2950A-2950N and a set of second sub-cores 2960A-2960N. In at least one embodiment, each sub-core in first sub-cores 2950A-2950N includes at least a first set of execution units 2952A-2952N and media/texture samplers 2954A-2954N. In at least one embodiment, each sub-core in second sub-cores 2960A-2960N includes at least a second set of execution units 2962A-2962N and samplers 2964A-2964N. In at least one embodiment, each sub-core 2950A-2950N, 2960A-2960N shares a set of shared resources 2970A-2970N. In at least one embodiment, shared resources include shared cache memory and pixel operation logic.

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, inference and/or training logic 115 may be used in graphics processor 2900 for inferencing or predicting operations based, at least in part, on weight parameters calculated using neural network training operations, neural network functions and/or architectures, or neural network use cases described herein.

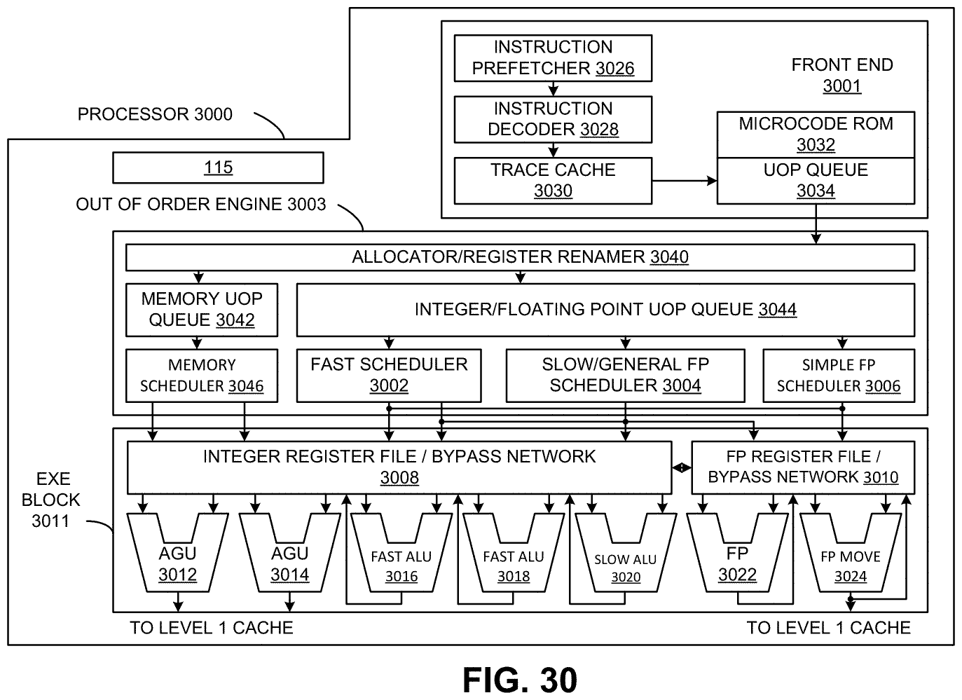

FIG. 30 is a block diagram illustrating micro-architecture for a processor 3000 that may include logic circuits to perform instructions, according to at least one embodiment. In at least one embodiment, processor 3000 may perform instructions, including x86 instructions, ARM instructions, specialized instructions for application-specific integrated circuits (ASICs), etc. In at least one embodiment, processor 3000 may include registers to store packed data, such as 64-bit wide MMX™ registers in microprocessors enabled with MMX technology from Intel Corporation of Santa Clara, Calif. In at least one embodiment, MMX registers, available in both integer and floating point forms, may operate with packed data elements that accompany single instruction, multiple data (“SIMD”) and streaming SIMD extensions (“SSE”) instructions. In at least one embodiment, 128-bit wide XMM registers relating to SSE2, SSE3, SSE4, AVX, or beyond (referred to generically as “SSEx”) technology may hold such packed data operands. In at least one embodiment, processor 3000 may perform instructions to accelerate machine learning or deep learning algorithms, training, or inferencing.

In at least one embodiment, processor 3000 includes an in-order front end (“front end”) 3001 to fetch instructions to be executed and prepare instructions to be used later in a processor pipeline. In at least one embodiment, front end 3001 may include several units. In at least one embodiment, an instruction prefetcher 3027 fetches instructions from memory and feeds instructions to an instruction decoder 3028 which in turn decodes or interprets instructions. For example, in at least one embodiment, instruction decoder 3028 decodes a received instruction into one or more operations called “micro-instructions” or “micro-operations” (also called “micro ops” or “uops”) that a machine may execute. In at least one embodiment, instruction decoder 3028 parses an instruction into an opcode and corresponding data and control fields that may be used by micro-architecture to perform operations in accordance with at least one embodiment. In at least one embodiment, a trace cache 3030 may assemble decoded uops into program ordered sequences or traces in a uop queue 3034 for execution. In at least one embodiment, when trace cache 3030 encounters a complex instruction, a microcode ROM 3032 provides uops needed to complete an operation.

In at least one embodiment, some instructions may be converted into a single micro-op, whereas others need several micro-ops to complete full operation. In at least one embodiment, if more than four micro-ops are needed to complete an instruction, instruction decoder 3028 may access microcode ROM 3032 to perform that instruction. In at least one embodiment, an instruction may be decoded into a small number of micro-ops for processing at instruction decoder 3028. In at least one embodiment, an instruction may be stored within microcode ROM 3032 should a number of micro-ops be needed to accomplish such operation. In at least one embodiment, trace cache 3030 refers to an entry point programmable logic array (“PLA”) to determine a correct micro-instruction pointer for reading microcode sequences to complete one or more instructions from microcode ROM 3032 in accordance with at least one embodiment. In at least one embodiment, after microcode ROM 3032 finishes sequencing micro-ops for an instruction, front end 3001 of a machine may resume fetching micro-ops from trace cache 3030.

In at least one embodiment, out-of-order execution engine (“out of order engine”) 3003 may prepare instructions for execution. In at least one embodiment, out-of-order execution logic has a number of buffers to smooth out and re-order flow of instructions to optimize performance as they go down a pipeline and get scheduled for execution. In at least one embodiment, out-of-order execution engine 3003 includes, without limitation, an allocator/register renamer 3040, a memory uop queue 3042, an integer/floating point uop queue 3044, a memory scheduler 3046, a fast scheduler 3002, a slow/general floating point scheduler (“slow/general FP scheduler”) 3004, and a simple floating point scheduler (“simple FP scheduler”) 3006. In at least one embodiment, fast schedule 3002, slow/general floating point scheduler 3004, and simple floating point scheduler 3006 are also collectively referred to herein as “uop schedulers 3002, 3004, 3006.” In at least one embodiment, allocator/register renamer 3040 allocates machine buffers and resources that each uop needs in order to execute. In at least one embodiment, allocator/register renamer 3040 renames logic registers onto entries in a register file. In at least one embodiment, allocator/register renamer 3040 also allocates an entry for each uop in one of two uop queues, memory uop queue 3042 for memory operations and integer/floating point uop queue 3044 for non-memory operations, in front of memory scheduler 3046 and uop schedulers 3002, 3004, 3006. In at least one embodiment, uop schedulers 3002, 3004, 3006, determine when a uop is ready to execute based on readiness of their dependent input register operand sources and availability of execution resources uops need to complete their operation. In at least one embodiment, fast scheduler 3002 may schedule on each half of a main clock cycle while slow/general floating point scheduler 3004 and simple floating point scheduler 3006 may schedule once per main processor clock cycle. In at least one embodiment, uop schedulers 3002, 3004, 3006 arbitrate for dispatch ports to schedule uops for execution.

In at least one embodiment, execution block 3011 includes, without limitation, an integer register file/bypass network 3008, a floating point register file/bypass network (“FP register file/bypass network”) 3010, address generation units (“AGUs”) 3012 and 3014, fast Arithmetic Logic Units (ALUs) (“fast ALUs”) 3016 and 3018, a slow Arithmetic Logic Unit (“slow ALU”) 3020, a floating point ALU (“FP”) 3022, and a floating point move unit (“FP move”) 3024. In at least one embodiment, integer register file/bypass network 3008 and floating point register file/bypass network 3010 are also referred to herein as “register files 3008, 3010.” In at least one embodiment, AGUSs 3012 and 3014, fast ALUs 3016 and 3018, slow ALU 3020, floating point ALU 3022, and floating point move unit 3024 are also referred to herein as “execution units 3012, 3014, 3016, 3018, 3020, 3022, and 3024.” In at least one embodiment, execution block 3011 may include, without limitation, any number (including zero) and type of register files, bypass networks, address generation units, and execution units, in any combination.

In at least one embodiment, register networks 3008, 3010 may be arranged between uop schedulers 3002, 3004, 3006, and execution units 3012, 3014, 3016, 3018, 3020, 3022, and 3024. In at least one embodiment, integer register file/bypass network 3008 performs integer operations. In at least one embodiment, floating point register file/bypass network 3010 performs floating point operations. In at least one embodiment, each of register networks 3008, 3010 may include, without limitation, a bypass network that may bypass or forward just completed results that have not yet been written into a register file to new dependent uops. In at least one embodiment, register networks 3008, 3010 may communicate data with each other. In at least one embodiment, integer register file/bypass network 3008 may include, without limitation, two separate register files, one register file for a low-order thirty-two bits of data and a second register file for a high order thirty-two bits of data. In at least one embodiment, floating point register file/bypass network 3010 may include, without limitation, 128-bit wide entries because floating point instructions typically have operands from 64 to 128 bits in width.

In at least one embodiment, execution units 3012, 3014, 3016, 3018, 3020, 3022, 3024 may execute instructions. In at least one embodiment, register networks 3008, 3010 store integer and floating point data operand values that micro-instructions need to execute. In at least one embodiment, processor 3000 may include, without limitation, any number and combination of execution units 3012, 3014, 3016, 3018, 3020, 3022, 3024. In at least one embodiment, floating point ALU 3022 and floating point move unit 3024, may execute floating point, MMX, SIMD, AVX and SSE, or other operations, including specialized machine learning instructions. In at least one embodiment, floating point ALU 3022 may include, without limitation, a 64-bit by 64-bit floating point divider to execute divide, square root, and remainder micro ops. In at least one embodiment, instructions involving a floating point value may be handled with floating point hardware. In at least one embodiment, ALU operations may be passed to fast ALUs 3016, 3018. In at least one embodiment, fast ALUS 3016, 3018 may execute fast operations with an effective latency of half a clock cycle. In at least one embodiment, most complex integer operations go to slow ALU 3020 as slow ALU 3020 may include, without limitation, integer execution hardware for long-latency type of operations, such as a multiplier, shifts, flag logic, and branch processing. In at least one embodiment, memory load/store operations may be executed by AGUs 3012, 3014. In at least one embodiment, fast ALU 3016, fast ALU 3018, and slow ALU 3020 may perform integer operations on 64-bit data operands. In at least one embodiment, fast ALU 3016, fast ALU 3018, and slow ALU 3020 may be implemented to support a variety of data bit sizes including sixteen, thirty-two, 128, 256, etc. In at least one embodiment, floating point ALU 3022 and floating point move unit 3024 may be implemented to support a range of operands having bits of various widths, such as 128-bit wide packed data operands in conjunction with SIMD and multimedia instructions.

In at least one embodiment, uop schedulers 3002, 3004, 3006 dispatch dependent operations before a parent load has finished executing. In at least one embodiment, as uops may be speculatively scheduled and executed in processor 3000, processor 3000 may also include logic to handle memory misses. In at least one embodiment, if a data load misses in a data cache, there may be dependent operations in flight in a pipeline that have left a scheduler with temporarily incorrect data. In at least one embodiment, a replay mechanism tracks and re-executes instructions that use incorrect data. In at least one embodiment, dependent operations might need to be replayed and independent ones may be allowed to complete. In at least one embodiment, schedulers and a replay mechanism of at least one embodiment of a processor may also be designed to catch instruction sequences for text string comparison operations.

In at least one embodiment, “registers” may refer to on-board processor storage locations that may be used as part of instructions to identify operands. In at least one embodiment, registers may be those that may be usable from outside of a processor (from a programmer's perspective). In at least one embodiment, registers might not be limited to a particular type of circuit. Rather, in at least one embodiment, a register may store data, provide data, and perform functions described herein. In at least one embodiment, registers described herein may be implemented by circuitry within a processor using any number of different techniques, such as dedicated physical registers, dynamically allocated physical registers using register renaming, combinations of dedicated and dynamically allocated physical registers, etc. In at least one embodiment, integer registers store 32-bit integer data. A register file of at least one embodiment also contains eight multimedia SIMD registers for packed data.

Inference and/or training logic 115 are used to perform inferencing and/or training operations associated with one or more embodiments. Details regarding inference and/or training logic 115 are provided herein in conjunction with FIGS. 1A and/or 1B. In at least one embodiment portions or all of inference and/or training logic 115 may be incorporated into execution block 3011 and other memory or registers shown or not shown. For example, in at least one embodiment, training and/or inferencing techniques described herein may use one or more of ALUs illustrated in execution block 3011. Moreover, weight parameters may be stored in on-chip or off-chip memory and/or registers (shown or not shown) that configure ALUs of execution block 3011 to perform one or more machine learning algorithms, neural network architectures, use cases, or training techniques described herein.

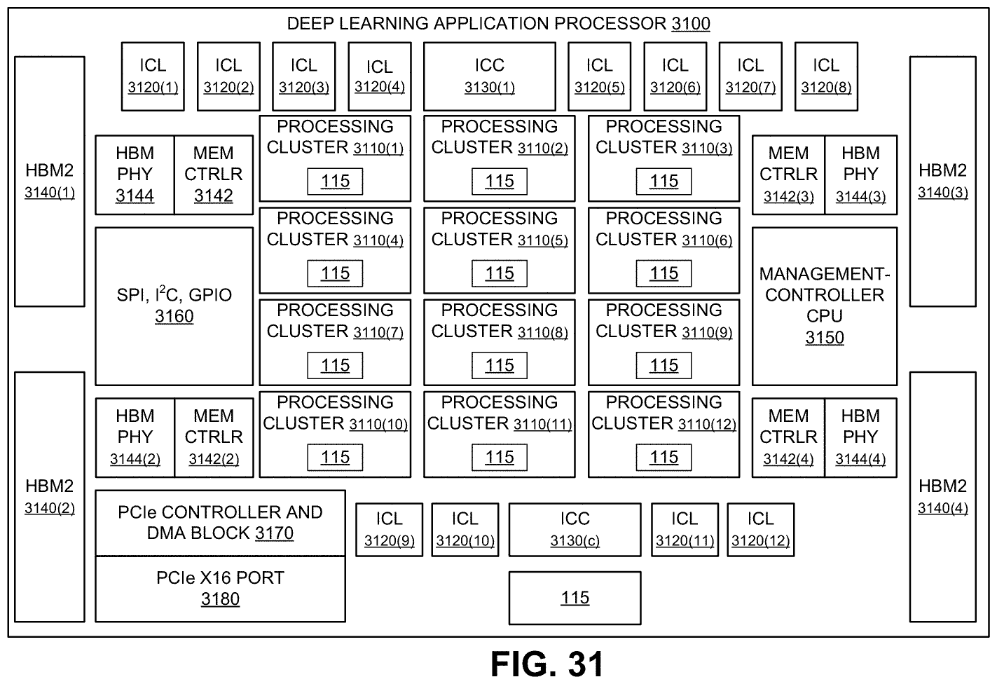

FIG. 31 illustrates a deep learning application processor 3100, according to at least one embodiment. In at least one embodiment, deep learning application processor 3100 uses instructions that, if executed by deep learning application processor 3100, cause deep learning application processor 3100 to perform some or all of processes and techniques described throughout this disclosure. In at least one embodiment, deep learning application processor 3100 is an application-specific integrated circuit (ASIC). In at least one embodiment, application processor 3100 performs matrix multiply operations either “hard-wired” into hardware as a result of performing one or more instructions or both. In at least one embodiment, deep learning application processor 3100 includes, without limitation, processing clusters 3110(1)-3110(12), Inter-Chip Links (“ICLs”) 3120(1)-3120(12), Inter-Chip Controllers (“ICCs”) 3130(1)-3130(2), high-bandwidth memory second generation (“HBM2”) 3140(1)-3140(4), memory controllers (“Mem Ctrlrs”) 3142(1)-3142(4), high bandwidth memory physical layer (“HBM PHY”) 3144(1)-3144(4), a management-controller central processing unit (“management-controller CPU”) 3150, a Serial Peripheral Interface, Inter-Integrated Circuit, and General Purpose Input/Output block (“SPI, I²C, GPIO”) 3160, a peripheral component interconnect express controller and direct memory access block (“PCIe Controller and DMA”) 3170, and a sixteen-lane peripheral component interconnect express port (“PCI Express ×16”) 3180.

In at least one embodiment, processing clusters 3110 may perform deep learning operations, including inference or prediction operations based on weight parameters calculated one or more training techniques, including those described herein. In at least one embodiment, each processing cluster 3110 may include, without limitation, any number and type of processors. In at least one embodiment, deep learning application processor 3100 may include any number and type of processing clusters. In at least one embodiment, Inter-Chip Links 3120 are bi-directional. In at least one embodiment, Inter-Chip Links 3120 and Inter-Chip Controllers 3130 enable multiple deep learning application processors 3100 to exchange information, including activation information resulting from performing one or more machine learning algorithms embodied in one or more neural networks. In at least one embodiment, deep learning application processor 3100 may include any number (including zero) and type of ICLs 3120 and ICCs 3131.

In at least one embodiment, HBM2s 3140 provide a total of 32 Gigabytes (GB) of memory. In at least one embodiment, HBM2 3140(i) is associated with both memory controller 3142(i) and HBM PHY 3144(i) where “i” is an arbitrary integer. In at least one embodiment, any number of HBM2s 3140 may provide any type and total amount of high bandwidth memory and may be associated with any number (including zero) and type of memory controllers 3142 and HBM PHYs 3144. In at least one embodiment, SPI, I²C, GPIO 3160, PCIe Controller and DMA 3170, and/or PCIe 3180 may be replaced with any number and type of blocks that enable any number and type of communication standards in any technically feasible fashion.

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, deep learning application processor is used to train a machine learning model, such as a neural network, to predict or infer information provided to deep learning application processor 3100. In at least one embodiment, deep learning application processor 3100 is used to infer or predict information based on a trained machine learning model (e.g., neural network) that has been trained by another processor or system or by deep learning application processor 3100. In at least one embodiment, processor 3100 may be used to perform one or more neural network use cases described herein.

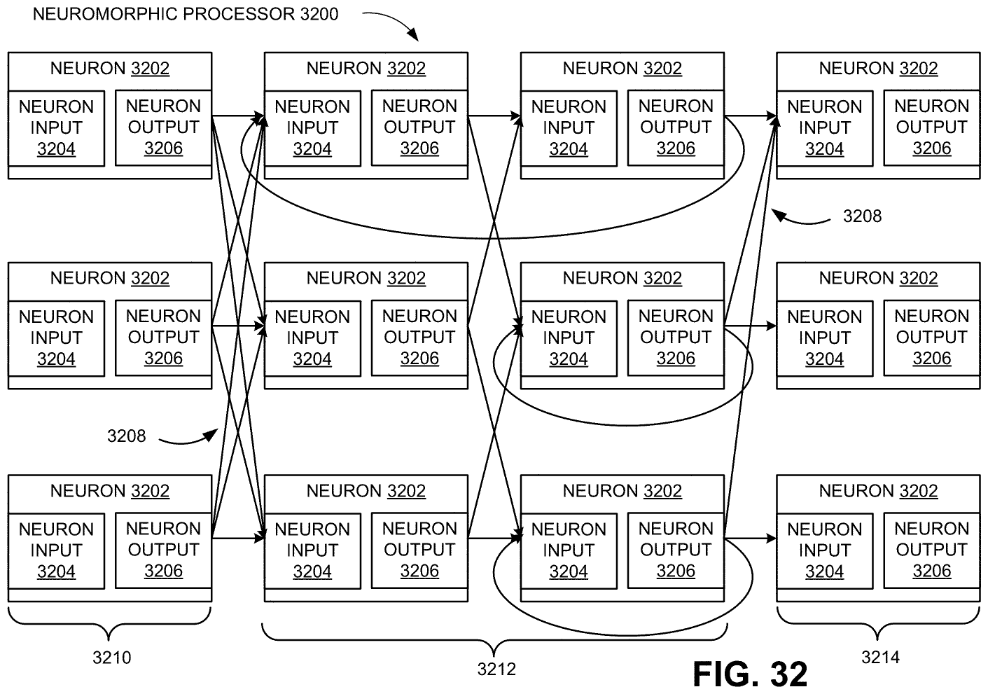

FIG. 32 is a block diagram of a neuromorphic processor 3200, according to at least one embodiment. In at least one embodiment, neuromorphic processor 3200 may receive one or more inputs from sources external to neuromorphic processor 3200. In at least one embodiment, these inputs may be transmitted to one or more neurons 3202 within neuromorphic processor 3200. In at least one embodiment, neurons 3202 and components thereof may be implemented using circuitry or logic, including one or more arithmetic logic units (ALUs). In at least one embodiment, neuromorphic processor 3200 may include, without limitation, thousands or millions of instances of neurons 3202, but any suitable number of neurons 3202 may be used. In at least one embodiment, each instance of neuron 3202 may include a neuron input 3204 and a neuron output 3206. In at least one embodiment, neurons 3202 may generate outputs that may be transmitted to inputs of other instances of neurons 3202. For example, in at least one embodiment, neuron inputs 3204 and neuron outputs 3206 may be interconnected via synapses 3208.

In at least one embodiment, neurons 3202 and synapses 3208 may be interconnected such that neuromorphic processor 3200 operates to process or analyze information received by neuromorphic processor 3200. In at least one embodiment, neurons 3202 may transmit an output pulse (or “fire” or “spike”) when inputs received through neuron input 3204 exceed a threshold. In at least one embodiment, neurons 3202 may sum or integrate signals received at neuron inputs 3204. For example, in at least one embodiment, neurons 3202 may be implemented as leaky integrate-and-fire neurons, wherein if a sum (referred to as a “membrane potential”) exceeds a threshold value, neuron 3202 may generate an output (or “fire”) using a transfer function such as a sigmoid or threshold function. In at least one embodiment, a leaky integrate-and-fire neuron may sum signals received at neuron inputs 3204 into a membrane potential and may also apply a decay factor (or leak) to reduce a membrane potential. In at least one embodiment, a leaky integrate-and-fire neuron may fire if multiple input signals are received at neuron inputs 3204 rapidly enough to exceed a threshold value (i.e., before a membrane potential decays too low to fire). In at least one embodiment, neurons 3202 may be implemented using circuits or logic that receive inputs, integrate inputs into a membrane potential, and decay a membrane potential. In at least one embodiment, inputs may be averaged, or any other suitable transfer function may be used. Furthermore, in at least one embodiment, neurons 3202 may include, without limitation, comparator circuits or logic that generate an output spike at neuron output 3206 when result of applying a transfer function to neuron input 3204 exceeds a threshold. In at least one embodiment, once neuron 3202 fires, it may disregard previously received input information by, for example, resetting a membrane potential to 0 or another suitable default value. In at least one embodiment, once membrane potential is reset to 0, neuron 3202 may resume normal operation after a suitable period of time (or refractory period).

In at least one embodiment, neurons 3202 may be interconnected through synapses 3208. In at least one embodiment, synapses 3208 may operate to transmit signals from an output of a first neuron 3202 to an input of a second neuron 3202. In at least one embodiment, neurons 3202 may transmit information over more than one instance of synapse 3208. In at least one embodiment, one or more instances of neuron output 3206 may be connected, via an instance of synapse 3208, to an instance of neuron input 3204 in same neuron 3202. In at least one embodiment, an instance of neuron 3202 generating an output to be transmitted over an instance of synapse 3208 may be referred to as a “pre-synaptic neuron” with respect to that instance of synapse 3208. In at least one embodiment, an instance of neuron 3202 receiving an input transmitted over an instance of synapse 3208 may be referred to as a “post-synaptic neuron” with respect to that instance of synapse 3208. Because an instance of neuron 3202 may receive inputs from one or more instances of synapse 3208, and may also transmit outputs over one or more instances of synapse 3208, a single instance of neuron 3202 may therefore be both a “pre-synaptic neuron” and “post-synaptic neuron,” with respect to various instances of synapses 3208, in at least one embodiment.

In at least one embodiment, neurons 3202 may be organized into one or more layers. In at least one embodiment, each instance of neuron 3202 may have one neuron output 3206 that may fan out through one or more synapses 3208 to one or more neuron inputs 3204. In at least one embodiment, neuron outputs 3206 of neurons 3202 in a first layer 3210 may be connected to neuron inputs 3204 of neurons 3202 in a second layer 3212. In at least one embodiment, layer 3210 may be referred to as a “feed-forward layer.” In at least one embodiment, each instance of neuron 3202 in an instance of first layer 3210 may fan out to each instance of neuron 3202 in second layer 3212. In at least one embodiment, first layer 3210 may be referred to as a “fully connected feed-forward layer.” In at least one embodiment, each instance of neuron 3202 in an instance of second layer 3212 may fan out to fewer than all instances of neuron 3202 in a third layer 3214. In at least one embodiment, second layer 3212 may be referred to as a “sparsely connected feed-forward layer.” In at least one embodiment, neurons 3202 in second layer 3212 may fan out to neurons 3202 in multiple other layers, including to neurons 3202 also in second layer 3212. In at least one embodiment, second layer 3212 may be referred to as a “recurrent layer.” In at least one embodiment, neuromorphic processor 3200 may include, without limitation, any suitable combination of recurrent layers and feed-forward layers, including, without limitation, both sparsely connected feed-forward layers and fully connected feed-forward layers.

In at least one embodiment, neuromorphic processor 3200 may include, without limitation, a reconfigurable interconnect architecture or dedicated hard-wired interconnects to connect synapse 3208 to neurons 3202. In at least one embodiment, neuromorphic processor 3200 may include, without limitation, circuitry or logic that allows synapses to be allocated to different neurons 3202 as needed based on neural network topology and neuron fan-in/out. For example, in at least one embodiment, synapses 3208 may be connected to neurons 3202 using an interconnect fabric, such as network-on-chip, or with dedicated connections. In at least one embodiment, synapse interconnections and components thereof may be implemented using circuitry or logic.

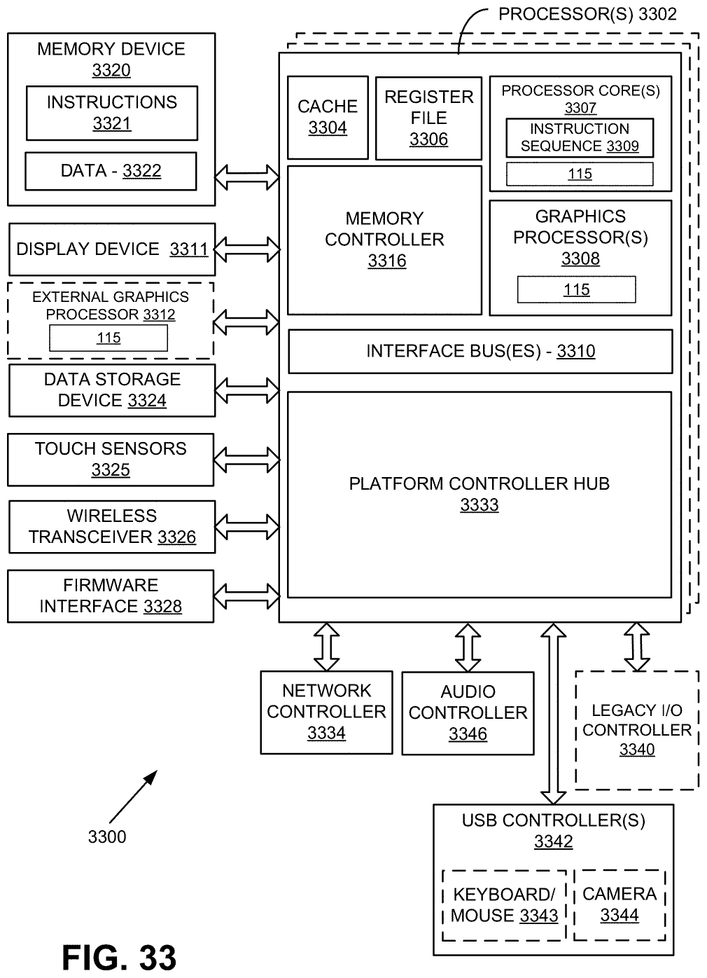

FIG. 33 is a block diagram of a processing system, according to at least one embodiment. In at least one embodiment, system 3300 includes one or more processors 3302 and one or more graphics processors 3308, and may be a single processor desktop system, a multiprocessor workstation system, or a server system having a large number of processors 3302 or processor cores 3307. In at least one embodiment, system 3300 is a processing platform incorporated within a system-on-a-chip (SoC) integrated circuit for use in mobile, handheld, or embedded devices.

In at least one embodiment, system 3300 can include, or be incorporated within a server-based gaming platform, a game console, including a game and media console, a mobile gaming console, a handheld game console, or an online game console. In at least one embodiment, system 3300 is a mobile phone, a smart phone, a tablet computing device or a mobile Internet device. In at least one embodiment, processing system 3300 can also include, couple with, or be integrated within a wearable device, such as a smart watch wearable device, a smart eyewear device, an augmented reality device, or a virtual reality device. In at least one embodiment, processing system 3300 is a television or set top box device having one or more processors 3302 and a graphical interface generated by one or more graphics processors 3308.

In at least one embodiment, one or more processors 3302 each include one or more processor cores 3307 to process instructions which, when executed, perform operations for system and user software. In at least one embodiment, each of one or more processor cores 3307 is configured to process a specific instruction sequence 3309. In at least one embodiment, instruction sequence 3309 may facilitate Complex Instruction Set Computing (CISC), Reduced Instruction Set Computing (RISC), or computing via a Very Long Instruction Word (VLIW). In at least one embodiment, processor cores 3307 may each process a different instruction sequence 3309, which may include instructions to facilitate emulation of other instruction sequences. In at least one embodiment, processor core 3307 may also include other processing devices, such a Digital Signal Processor (DSP).

In at least one embodiment, processor 3302 includes a cache memory 3304. In at least one embodiment, processor 3302 can have a single internal cache or multiple levels of internal cache. In at least one embodiment, cache memory is shared among various components of processor 3302. In at least one embodiment, processor 3302 also uses an external cache (e.g., a Level-3 (L3) cache or Last Level Cache (LLC)) (not shown), which may be shared among processor cores 3307 using known cache coherency techniques. In at least one embodiment, a register file 3306 is additionally included in processor 3302, which may include different types of registers for storing different types of data (e.g., integer registers, floating point registers, status registers, and an instruction pointer register). In at least one embodiment, register file 3306 may include general-purpose registers or other registers.

In at least one embodiment, one or more processor(s) 3302 are coupled with one or more interface bus(es) 3310 to transmit communication signals such as address, data, or control signals between processor 3302 and other components in system 3300. In at least one embodiment, interface bus 3310 can be a processor bus, such as a version of a Direct Media Interface (DMI) bus. In at least one embodiment, interface bus 3310 is not limited to a DMI bus, and may include one or more Peripheral Component Interconnect buses (e.g., PCI, PCI Express), memory busses, or other types of interface busses. In at least one embodiment processor(s) 3302 include an integrated memory controller 3316 and a platform controller hub 3330. In at least one embodiment, memory controller 3316 facilitates communication between a memory device and other components of system 3300, while platform controller hub (PCH) 3330 provides connections to I/O devices via a local I/O bus.

In at least one embodiment, a memory device 3320 can be a dynamic random access memory (DRAM) device, a static random access memory (SRAM) device, flash memory device, phase-change memory device, or some other memory device having suitable performance to serve as process memory. In at least one embodiment, memory device 3320 can operate as system memory for system 3300, to store data 3322 and instructions 3321 for use when one or more processors 3302 executes an application or process. In at least one embodiment, memory controller 3316 also couples with an optional external graphics processor 3312, which may communicate with one or more graphics processors 3308 in processors 3302 to perform graphics and media operations. In at least one embodiment, a display device 3311 can connect to processor(s) 3302. In at least one embodiment, display device 3311 can include one or more of an internal display device, as in a mobile electronic device or a laptop device, or an external display device attached via a display interface (e.g., DisplayPort, etc.). In at least one embodiment, display device 3311 can include a head mounted display (HMD) such as a stereoscopic display device for use in virtual reality (VR) applications or augmented reality (AR) applications.

In at least one embodiment, platform controller hub 3330 enables peripherals to connect to memory device 3320 and processor 3302 via a high-speed I/O bus. In at least one embodiment, I/O peripherals include, but are not limited to, an audio controller 3346, a network controller 3334, a firmware interface 3328, a wireless transceiver 3327, touch sensors 3325, a data storage device 3324 (e.g., hard disk drive, flash memory, etc.). In at least one embodiment, data storage device 3324 can connect via a storage interface (e.g., SATA) or via a peripheral bus, such as a Peripheral Component Interconnect bus (e.g., PCI, PCI Express). In at least one embodiment, touch sensors 3325 can include touch screen sensors, pressure sensors, or fingerprint sensors. In at least one embodiment, wireless transceiver 3327 can be a Wi-Fi transceiver, a Bluetooth transceiver, or a mobile network transceiver such as a 3G, 4G, or Long Term Evolution (LTE) transceiver. In at least one embodiment, firmware interface 3328 enables communication with system firmware, and can be, for example, a unified extensible firmware interface (UEFI). In at least one embodiment, network controller 3334 can enable a network connection to a wired network. In at least one embodiment, a high-performance network controller (not shown) couples with interface bus 3310. In at least one embodiment, audio controller 3346 is a multi-channel high definition audio controller. In at least one embodiment, system 3300 includes an optional legacy I/O controller 3340 for coupling legacy (e.g., Personal System 2 (PS/2)) devices to system 3300. In at least one embodiment, platform controller hub 3330 can also connect to one or more Universal Serial Bus (USB) controllers 3342 connect input devices, such as keyboard and mouse 3343 combinations, a camera 3344, or other USB input devices.

In at least one embodiment, an instance of memory controller 3316 and platform controller hub 3330 may be integrated into a discreet external graphics processor, such as external graphics processor 3312. In at least one embodiment, platform controller hub 3330 and/or memory controller 3316 may be external to one or more processor(s) 3302. For example, in at least one embodiment, system 3300 can include an external memory controller 3316 and platform controller hub 3330, which may be configured as a memory controller hub and peripheral controller hub within a system chipset that is in communication with processor(s) 3302.

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, portions or all of inference and/or training logic 115 may be incorporated into system 3300. For example, in at least one embodiment, training and/or inferencing techniques described herein may use one or more of ALUs embodied in a 3D pipeline. Moreover, in at least one embodiment, inferencing and/or training operations described herein may be done using logic other than logic illustrated in FIG. 1A or 1B. In at least one embodiment, weight parameters may be stored in on-chip or off-chip memory and/or registers (shown or not shown) that configure ALUs of graphics processor 3308 to perform one or more machine learning algorithms, neural network architectures, use cases, or training techniques described herein.

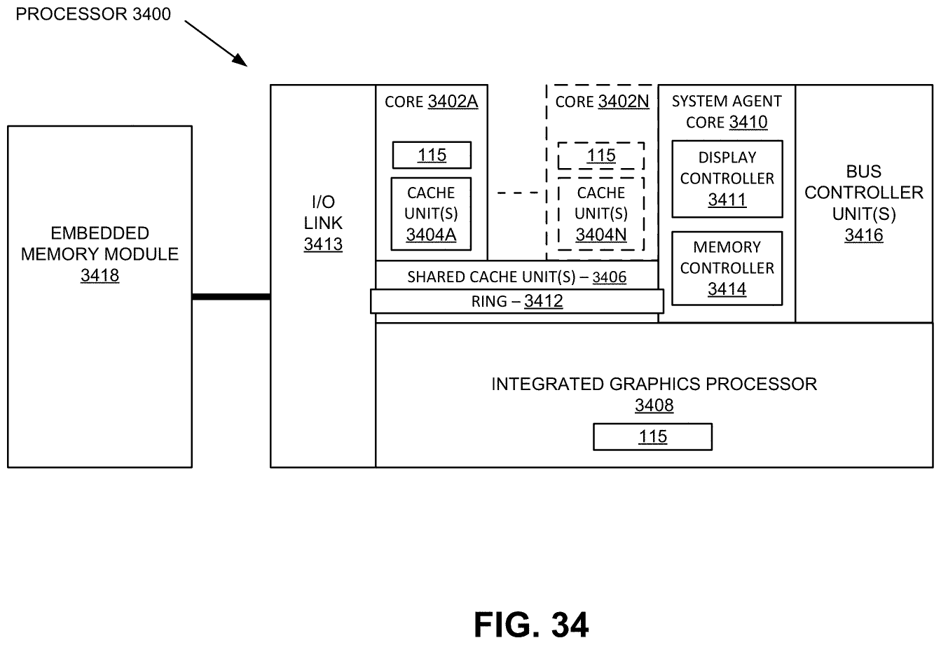

FIG. 34 is a block diagram of a processor 3400 having one or more processor cores 3402A-3402N, an integrated memory controller 3414, and an integrated graphics processor 3408, according to at least one embodiment. In at least one embodiment, processor 3400 can include additional cores up to and including additional core 3402N represented by dashed lined boxes. In at least one embodiment, each of processor cores 3402A-3402N includes one or more internal cache units 3404A-3404N. In at least one embodiment, each processor core also has access to one or more shared cached units 3406.

In at least one embodiment, internal cache units 3404A-3404N and shared cache units 3406 represent a cache memory hierarchy within processor 3400. In at least one embodiment, cache memory units 3404A-3404N may include at least one level of instruction and data cache within each processor core and one or more levels of shared mid-level cache, such as a Level 2 (L2), Level 3 (L3), Level 4 (L4), or other levels of cache, where a highest level of cache before external memory is classified as an LLC. In at least one embodiment, cache coherency logic maintains coherency between various cache units 3406 and 3404A-3404N.

In at least one embodiment, processor 3400 may also include a set of one or more bus controller units 3416 and a system agent core 3410. In at least one embodiment, bus controller units 3416 manage a set of peripheral buses, such as one or more PCI or PCI express busses. In at least one embodiment, system agent core 3410 provides management functionality for various processor components. In at least one embodiment, system agent core 3410 includes one or more integrated memory controllers 3414 to manage access to various external memory devices (not shown).

In at least one embodiment, one or more of processor cores 3402A-3402N include support for simultaneous multi-threading. In at least one embodiment, system agent core 3410 includes components for coordinating and operating cores 3402A-3402N during multi-threaded processing. In at least one embodiment, system agent core 3410 may additionally include a power control unit (PCU), which includes logic and components to regulate one or more power states of processor cores 3402A-3402N and graphics processor 3408.

In at least one embodiment, processor 3400 additionally includes graphics processor 3408 to execute graphics processing operations. In at least one embodiment, graphics processor 3408 couples with shared cache units 3406, and system agent core 3410, including one or more integrated memory controllers 3414. In at least one embodiment, system agent core 3410 also includes a display controller 3411 to drive graphics processor output to one or more coupled displays. In at least one embodiment, display controller 3411 may also be a separate module coupled with graphics processor 3408 via at least one interconnect, or may be integrated within graphics processor 3408.

In at least one embodiment, a ring-based interconnect unit 3412 is used to couple internal components of processor 3400. In at least one embodiment, an alternative interconnect unit may be used, such as a point-to-point interconnect, a switched interconnect, or other techniques. In at least one embodiment, graphics processor 3408 couples with ring interconnect 3412 via an I/O link 3413.

In at least one embodiment, I/O link 3413 represents at least one of multiple varieties of I/O interconnects, including an on package I/O interconnect which facilitates communication between various processor components and a high-performance embedded memory module 3418, such as an eDRAM module. In at least one embodiment, each of processor cores 3402A-3402N and graphics processor 3408 use embedded memory module 3418 as a shared Last Level Cache.

In at least one embodiment, processor cores 3402A-3402N are homogeneous cores executing a common instruction set architecture. In at least one embodiment, processor cores 3402A-3402N are heterogeneous in terms of instruction set architecture (ISA), where one or more of processor cores 3402A-3402N execute a common instruction set, while one or more other cores of processor cores 3402A-3402N executes a subset of a common instruction set or a different instruction set. In at least one embodiment, processor cores 3402A-3402N are heterogeneous in terms of microarchitecture, where one or more cores having a relatively higher power consumption couple with one or more power cores having a lower power consumption. In at least one embodiment, processor 3400 can be implemented on one or more chips or as an SoC integrated circuit.

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, portions or all of inference and/or training logic 115 may be incorporated into graphics processor 3408. For example, in at least one embodiment, training and/or inferencing techniques described herein may use one or more of ALUs embodied in a 3D pipeline, graphics core(s) 3402, shared function logic, or other logic in FIG. 34. Moreover, in at least one embodiment, inferencing and/or training operations described herein may be done using logic other than logic illustrated in FIG. 1A or 1B. In at least one embodiment, weight parameters may be stored in on-chip or off-chip memory and/or registers (shown or not shown) that configure ALUs of processor 3400 to perform one or more machine learning algorithms, neural network architectures, use cases, or training techniques described herein.

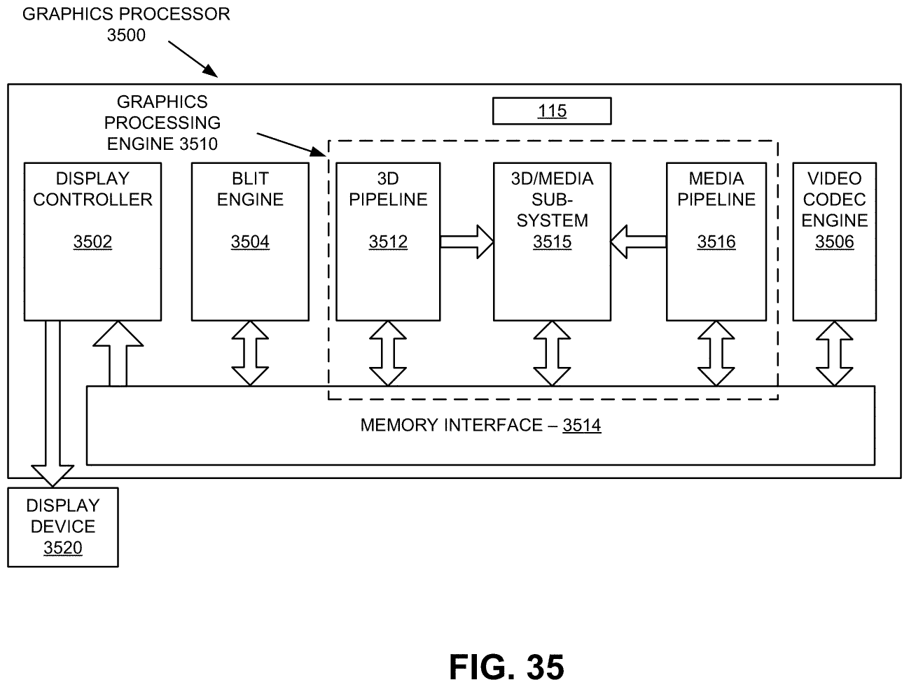

FIG. 35 is a block diagram of a graphics processor 3500, which may be a discrete graphics processing unit, or may be a graphics processor integrated with a plurality of processing cores. In at least one embodiment, graphics processor 3500 communicates via a memory mapped I/O interface to registers on graphics processor 3500 and with commands placed into memory. In at least one embodiment, graphics processor 3500 includes a memory interface 3514 to access memory. In at least one embodiment, memory interface 3514 is an interface to local memory, one or more internal caches, one or more shared external caches, and/or to system memory.

In at least one embodiment, graphics processor 3500 also includes a display controller 3502 to drive display output data to a display device 3520. In at least one embodiment, display controller 3502 includes hardware for one or more overlay planes for display device 3520 and composition of multiple layers of video or user interface elements. In at least one embodiment, display device 3520 can be an internal or external display device. In at least one embodiment, display device 3520 is a head mounted display device, such as a virtual reality (VR) display device or an augmented reality (AR) display device. In at least one embodiment, graphics processor 3500 includes a video codec engine 3506 to encode, decode, or transcode media to, from, or between one or more media encoding formats, including, but not limited to Moving Picture Experts Group (MPEG) formats such as MPEG-2, Advanced Video Coding (AVC) formats such as H.274/MPEG-4 AVC, as well as Society of Motion Picture & Television Engineers (SMPTE) 421M/VC-1, and Joint Photographic Experts Group (JPEG) formats such as JPEG, and Motion JPEG (MJPEG) formats.

In at least one embodiment, graphics processor 3500 includes a block image transfer (BLIT) engine 3504 to perform two-dimensional (2D) rasterizer operations including, for example, bit-boundary block transfers. However, in at least one embodiment, 2D graphics operations are performed using one or more components of a graphics processing engine (GPE) 3510. In at least one embodiment, GPE 3510 is a compute engine for performing graphics operations, including three-dimensional (3D) graphics operations and media operations.

In at least one embodiment, GPE 3510 includes a 3D pipeline 3512 for performing 3D operations, such as rendering three-dimensional images and scenes using processing functions that act upon 3D primitive shapes (e.g., rectangle, triangle, etc.). In at least one embodiment, 3D pipeline 3512 includes programmable and fixed function elements that perform various tasks and/or spawn execution threads to a 3D/Media sub-system 3515. While 3D pipeline 3512 can be used to perform media operations, in at least one embodiment, GPE 3510 also includes a media pipeline 3516 that is used to perform media operations, such as video post-processing and image enhancement.

In at least one embodiment, media pipeline 3516 includes fixed function or programmable logic units to perform one or more specialized media operations, such as video decode acceleration, video de-interlacing, and video encode acceleration in place of, or on behalf of, video codec engine 3506. In at least one embodiment, media pipeline 3516 additionally includes a thread spawning unit to spawn threads for execution on 3D/Media sub-system 3515. In at least one embodiment, spawned threads perform computations for media operations on one or more graphics execution units included in 3D/Media sub-system 3515.

In at least one embodiment, 3D/Media subsystem 3515 includes logic for executing threads spawned by 3D pipeline 3512 and media pipeline 3516. In at least one embodiment, 3D pipeline 3512 and media pipeline 3516 send thread execution requests to 3D/Media subsystem 3515, which includes thread dispatch logic for arbitrating and dispatching various requests to available thread execution resources. In at least one embodiment, execution resources include an array of graphics execution units to process 3D and media threads. In at least one embodiment, 3D/Media subsystem 3515 includes one or more internal caches for thread instructions and data. In at least one embodiment, subsystem 3515 also includes shared memory, including registers and addressable memory, to share data between threads and to store output data.

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, portions or all of inference and/or training logic 115 may be incorporated into graphics processor 3500. For example, in at least one embodiment, training and/or inferencing techniques described herein may use one or more of ALUs embodied in 3D pipeline 3512. Moreover, in at least one embodiment, inferencing and/or training operations described herein may be done using logic other than logic illustrated in FIG. 1A or 1B. In at least one embodiment, weight parameters may be stored in on-chip or off-chip memory and/or registers (shown or not shown) that configure ALUs of graphics processor 3500 to perform one or more machine learning algorithms, neural network architectures, use cases, or training techniques described herein.

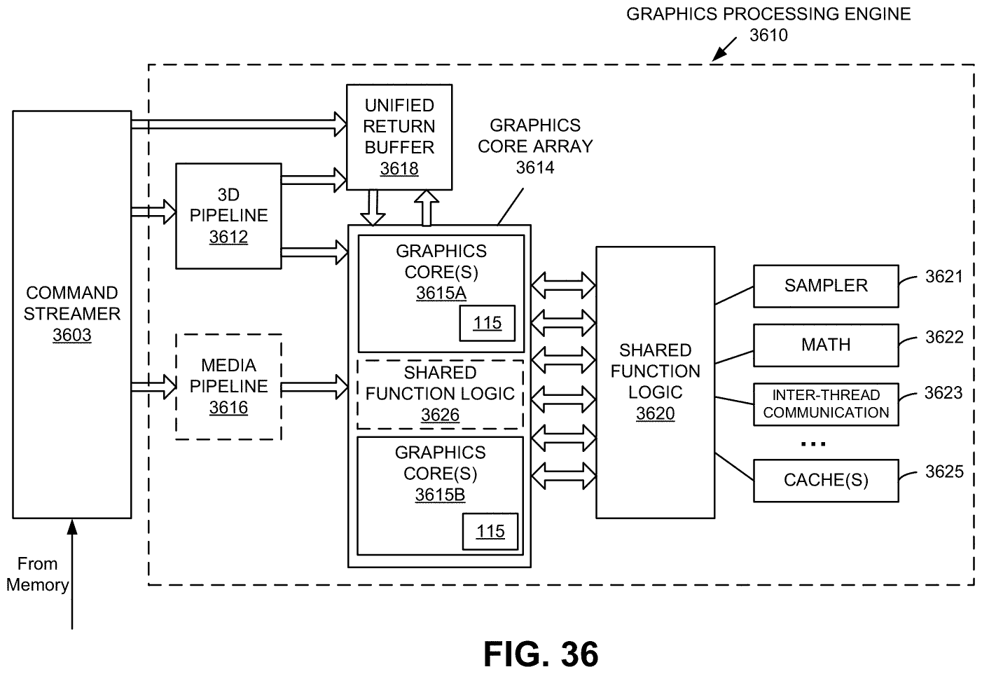

FIG. 36 is a block diagram of a graphics processing engine 3610 of a graphics processor in accordance with at least one embodiment. In at least one embodiment, graphics processing engine (GPE) 3610 is a version of GPE 3510 shown in FIG. 35. In at least one embodiment, a media pipeline 3616 is optional and may not be explicitly included within GPE 3610. In at least one embodiment, a separate media and/or image processor is coupled to GPE 3610.

In at least one embodiment, GPE 3610 is coupled to or includes a command streamer 3603, which provides a command stream to a 3D pipeline 3612 and/or media pipeline 3616. In at least one embodiment, command streamer 3603 is coupled to memory, which can be system memory, or one or more of internal cache memory and shared cache memory. In at least one embodiment, command streamer 3603 receives commands from memory and sends commands to 3D pipeline 3612 and/or media pipeline 3616. In at least one embodiment, commands are instructions, primitives, or micro-operations fetched from a ring buffer, which stores commands for 3D pipeline 3612 and media pipeline 3616. In at least one embodiment, a ring buffer can additionally include batch command buffers storing batches of multiple commands. In at least one embodiment, commands for 3D pipeline 3612 can also include references to data stored in memory, such as, but not limited to, vertex and geometry data for 3D pipeline 3612 and/or image data and memory objects for media pipeline 3616. In at least one embodiment, 3D pipeline 3612 and media pipeline 3616 process commands and data by performing operations or by dispatching one or more execution threads to a graphics core array 3614. In at least one embodiment, graphics core array 3614 includes one or more blocks of graphics cores (e.g., graphics core(s) 3615A, graphics core(s) 3615B), each block including one or more graphics cores. In at least one embodiment, each graphics core includes a set of graphics execution resources that includes general-purpose and graphics specific execution logic to perform graphics and compute operations, as well as fixed function texture processing and/or machine learning and artificial intelligence acceleration logic, including inference and/or training logic 115 in FIG. 1A and FIG. 1B.

In at least one embodiment, 3D pipeline 3612 includes fixed function and programmable logic to process one or more shader programs, such as vertex shaders, geometry shaders, pixel shaders, fragment shaders, compute shaders, or other shader programs, by processing instructions and dispatching execution threads to graphics core array 3614. In at least one embodiment, graphics core array 3614 provides a unified block of execution resources for use in processing shader programs. In at least one embodiment, a multi-purpose execution logic (e.g., execution units) within graphics core(s) 3615A-3615B of graphic core array 3614 includes support for various 3D API shader languages and can execute multiple simultaneous execution threads associated with multiple shaders.

In at least one embodiment, graphics core array 3614 also includes execution logic to perform media functions, such as video and/or image processing. In at least one embodiment, execution units additionally include general-purpose logic that is programmable to perform parallel general-purpose computational operations, in addition to graphics processing operations.

In at least one embodiment, output data generated by threads executing on graphics core array 3614 can output data to memory in a unified return buffer (URB) 3618. In at least one embodiment, URB 3618 can store data for multiple threads. In at least one embodiment, URB 3618 may be used to send data between different threads executing on graphics core array 3614. In at least one embodiment, URB 3618 may additionally be used for synchronization between threads on graphics core array 3614 and fixed function logic within shared function logic 3620.

In at least one embodiment, graphics core array 3614 is scalable, such that graphics core array 3614 includes a variable number of graphics cores, each having a variable number of execution units based on a target power and performance level of GPE 3610. In at least one embodiment, execution resources are dynamically scalable, such that execution resources may be enabled or disabled as needed.

In at least one embodiment, graphics core array 3614 is coupled to shared function logic 3620 that includes multiple resources that are shared between graphics cores in graphics core array 3614. In at least one embodiment, shared functions performed by shared function logic 3620 are embodied in hardware logic units that provide specialized supplemental functionality to graphics core array 3614. In at least one embodiment, shared function logic 3620 includes but is not limited to a sampler unit 3621, a math unit 3622, and inter-thread communication (ITC) logic 3629. In at least one embodiment, one or more cache(s) 3625 are included in, or coupled to, shared function logic 3620.

In at least one embodiment, a shared function is used if demand for a specialized function is insufficient for inclusion within graphics core array 3614. In at least one embodiment, a single instantiation of a specialized function is used in shared function logic 3620 and shared among other execution resources within graphics core array 3614. In at least one embodiment, specific shared functions within shared function logic 3620 that are used extensively by graphics core array 3614 may be included within shared function logic 3620 within graphics core array 3614. In at least one embodiment, shared function logic 3620 within graphics core array 3614 can include some or all logic within shared function logic 3620. In at least one embodiment, all logic elements within shared function logic 3620 may be duplicated within shared function logic 3627 of graphics core array 3614. In at least one embodiment, shared function logic 3620 is excluded in favor of shared function logic 3627 within graphics core array 3614.

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, portions or all of inference and/or training logic 115 may be incorporated into graphics processor 3610. For example, in at least one embodiment, training and/or inferencing techniques described herein may use one or more of ALUs embodied in 3D pipeline 3612, graphics core(s) 3615, shared function logic 3626, shared function logic 3620, or other logic in FIG. 36. Moreover, in at least one embodiment, inferencing and/or training operations described herein may be done using logic other than logic illustrated in FIG. 1A or 1B. In at least one embodiment, weight parameters may be stored in on-chip or off-chip memory and/or registers (shown or not shown) that configure ALUs of graphics processor 3610 to perform one or more machine learning algorithms, neural network architectures, use cases, or training techniques described herein.

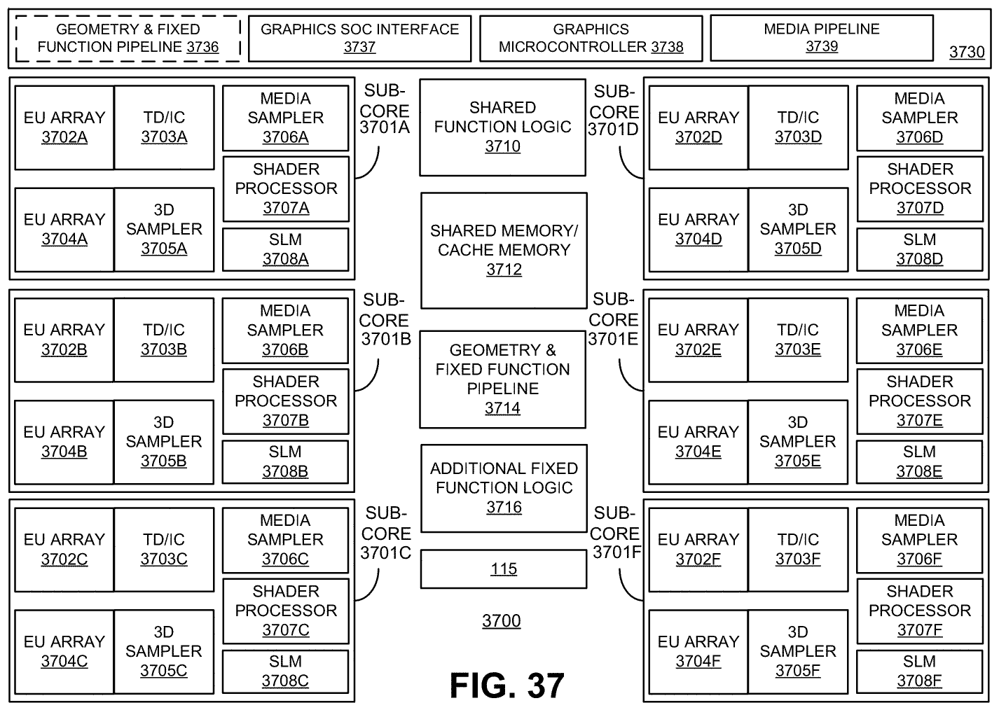

FIG. 37 is a block diagram of hardware logic of a graphics processor core 3700, according to at least one embodiment described herein. In at least one embodiment, graphics processor core 3700 is included within a graphics core array. In at least one embodiment, graphics processor core 3700, sometimes referred to as a core slice, can be one or multiple graphics cores within a modular graphics processor. In at least one embodiment, graphics processor core 3700 is exemplary of one graphics core slice, and a graphics processor as described herein may include multiple graphics core slices based on target power and performance envelopes. In at least one embodiment, each graphics processor core 3700 can include a fixed function block 3730 coupled with multiple sub-cores 3701A-3701F, also referred to as sub-slices, that include modular blocks of general-purpose and fixed function logic.

In at least one embodiment, fixed function block 3730 includes a geometry and fixed function pipeline 3736 that can be shared by all sub-cores in graphics processor 3700, for example, in lower performance and/or lower power graphics processor implementations. In at least one embodiment, geometry and fixed function pipeline 3736 includes a 3D fixed function pipeline, a video front-end unit, a thread spawner and thread dispatcher, and a unified return buffer manager, which manages unified return buffers.

In at least one embodiment, fixed function block 3730 also includes a graphics SoC interface 3737, a graphics microcontroller 3738, and a media pipeline 3739. In at least one embodiment, graphics SoC interface 3737 provides an interface between graphics processor core 3700 and other processor cores within a system on a chip integrated circuit. In at least one embodiment, graphics microcontroller 3738 is a programmable sub-processor that is configurable to manage various functions of graphics processor 3700, including thread dispatch, scheduling, and pre-emption. In at least one embodiment, media pipeline 3739 includes logic to facilitate decoding, encoding, pre-processing, and/or post-processing of multimedia data, including image and video data. In at least one embodiment, media pipeline 3739 implements media operations via requests to compute or sampling logic within sub-cores 3701A-3701F.

In at least one embodiment, SoC interface 3737 enables graphics processor core 3700 to communicate with general-purpose application processor cores (e.g., CPUs) and/or other components within an SoC, including memory hierarchy elements such as a shared last level cache memory, system RAM, and/or embedded on-chip or on-package DRAM. In at least one embodiment, SoC interface 3737 can also enable communication with fixed function devices within an SoC, such as camera imaging pipelines, and enables use of and/or implements global memory atomics that may be shared between graphics processor core 3700 and CPUs within an SoC. In at least one embodiment, graphics SoC interface 3737 can also implement power management controls for graphics processor core 3700 and enable an interface between a clock domain of graphics processor core 3700 and other clock domains within an SoC. In at least one embodiment, SoC interface 3737 enables receipt of command buffers from a command streamer and global thread dispatcher that are configured to provide commands and instructions to each of one or more graphics cores within a graphics processor. In at least one embodiment, commands and instructions can be dispatched to media pipeline 3739, when media operations are to be performed, or a geometry and fixed function pipeline (e.g., geometry and fixed function pipeline 3736, and/or a geometry and fixed function pipeline 3714) when graphics processing operations are to be performed.

In at least one embodiment, graphics microcontroller 3738 can be configured to perform various scheduling and management tasks for graphics processor core 3700. In at least one embodiment, graphics microcontroller 3738 can perform graphics and/or compute workload scheduling on various graphics parallel engines within execution unit (EU) arrays 3702A-3702F, 3704A-3704F within sub-cores 3701A-3701F. In at least one embodiment, host software executing on a CPU core of an SoC including graphics processor core 3700 can submit workloads to one of multiple graphic processor paths, which invokes a scheduling operation on an appropriate graphics engine. In at least one embodiment, scheduling operations include determining which workload to run next, submitting a workload to a command streamer, pre-empting existing workloads running on an engine, monitoring progress of a workload, and notifying host software when a workload is complete. In at least one embodiment, graphics microcontroller 3738 can also facilitate low-power or idle states for graphics processor core 3700, providing graphics processor core 3700 with an ability to save and restore registers within graphics processor core 3700 across low-power state transitions independently from an operating system and/or graphics driver software on a system.

In at least one embodiment, graphics processor core 3700 may have greater than or fewer than illustrated sub-cores 3701A-3701F, up to N modular sub-cores. For each set of N sub-cores, in at least one embodiment, graphics processor core 3700 can also include shared function logic 3710, shared and/or cache memory 3712, geometry/fixed function pipeline 3714, as well as additional fixed function logic 3716 to accelerate various graphics and compute processing operations. In at least one embodiment, shared function logic 3710 can include logic units (e.g., sampler, math, and/or inter-thread communication logic) that can be shared by each N sub-cores within graphics processor core 3700. In at least one embodiment, shared and/or cache memory 3712 can be a last-level cache for N sub-cores 3701A-3701F within graphics processor core 3700 and can also serve as shared memory that is accessible by multiple sub-cores. In at least one embodiment, geometry/fixed function pipeline 3714 can be included instead of geometry/fixed function pipeline 3736 within fixed function block 3730 and can include similar logic units.

In at least one embodiment, graphics processor core 3700 includes additional fixed function logic 3716 that can include various fixed function acceleration logic for use by graphics processor core 3700. In at least one embodiment, additional fixed function logic 3716 includes an additional geometry pipeline for use in position-only shading. In position-only shading, at least two geometry pipelines exist, whereas in a full geometry pipeline within geometry and fixed function pipelines 3714, 3736, and a cull pipeline, which is an additional geometry pipeline that may be included within additional fixed function logic 3714. In at least one embodiment, a cull pipeline is a trimmed down version of a full geometry pipeline. In at least one embodiment, a full pipeline and a cull pipeline can execute different instances of an application, each instance having a separate context. In at least one embodiment, position only shading can hide long cull runs of discarded triangles, enabling shading to be completed earlier in some instances. For example, in at least one embodiment, cull pipeline logic within additional fixed function logic 3716 can execute position shaders in parallel with a main application and generally generates critical results faster than a full pipeline, as a cull pipeline fetches and shades position attributes of vertices, without performing rasterization and rendering of pixels to a frame buffer. In at least one embodiment, a cull pipeline can use generated critical results to compute visibility information for all triangles without regard to whether those triangles are culled. In at least one embodiment, a full pipeline (which in this instance may be referred to as a replay pipeline) can consume visibility information to skip culled triangles to shade only visible triangles that are finally passed to a rasterization phase.

In at least one embodiment, additional fixed function logic 3716 can also include machine-learning acceleration logic, such as fixed function matrix multiplication logic, for implementations including optimizations for machine learning training or inferencing.

In at least one embodiment, within each graphics sub-core 3701A-3701F includes a set of execution resources that may be used to perform graphics, media, and compute operations in response to requests by graphics pipeline, media pipeline, or shader programs. In at least one embodiment, graphics sub-cores 3701A-3701F include multiple EU arrays 3702A-3702F, 3704A-3704F, thread dispatch and inter-thread communication (TD/IC) logic 3703A-3703F, a 3D (e.g., texture) sampler 3705A-3705F, a media sampler 3706A-3706F, a shader processor 3707A-3707F, and shared local memory (SLM) 3708A-3708F. In at least one embodiment, EU arrays 3702A-3702F, 3704A-3704F each include multiple execution units, which are general-purpose graphics processing units capable of performing floating-point and integer/fixed-point logic operations in service of a graphics, media, or compute operation, including graphics, media, or compute shader programs. In at least one embodiment, TD/IC logic 3703A-3703F performs local thread dispatch and thread control operations for execution units within a sub-core and facilitates communication between threads executing on execution units of a sub-core. In at least one embodiment, 3D samplers 3705A-3705F can read texture or other 3D graphics related data into memory. In at least one embodiment, 3D samplers can read texture data differently based on a configured sample state and texture format associated with a given texture. In at least one embodiment, media samplers 3706A-3706F can perform similar read operations based on a type and format associated with media data. In at least one embodiment, each graphics sub-core 3701A-3701F can alternately include a unified 3D and media sampler. In at least one embodiment, threads executing on execution units within each of sub-cores 3701A-3701F can make use of shared local memory 3708A-3708F within each sub-core, to enable threads executing within a thread group to execute using a common pool of on-chip memory.

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, portions or all of inference and/or training logic 115 may be incorporated into graphics processor core 3700. For example, in at least one embodiment, training and/or inferencing techniques described herein may use one or more of ALUs embodied in a 3D pipeline, graphics microcontroller 3738, geometry and fixed function pipeline 3714 and 3736, or other logic in FIG. 37. Moreover, in at least one embodiment, inferencing and/or training operations described herein may be done using logic other than logic illustrated in FIG. 1A or 1B. In at least one embodiment, weight parameters may be stored in on-chip or off-chip memory and/or registers (shown or not shown) that configure ALUs of graphics processor core 3700 to perform one or more machine learning algorithms, neural network architectures, use cases, or training techniques described herein.

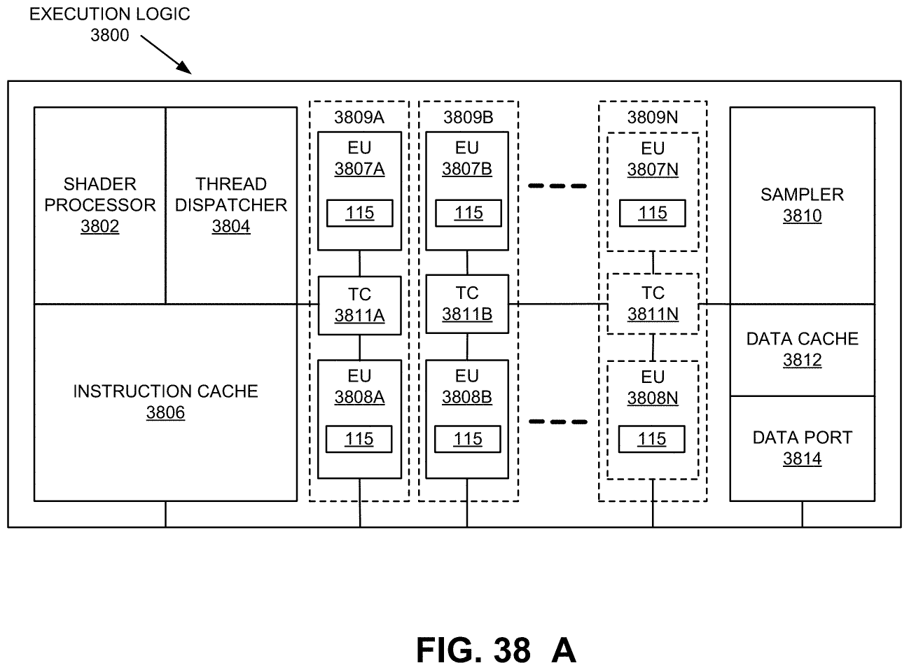

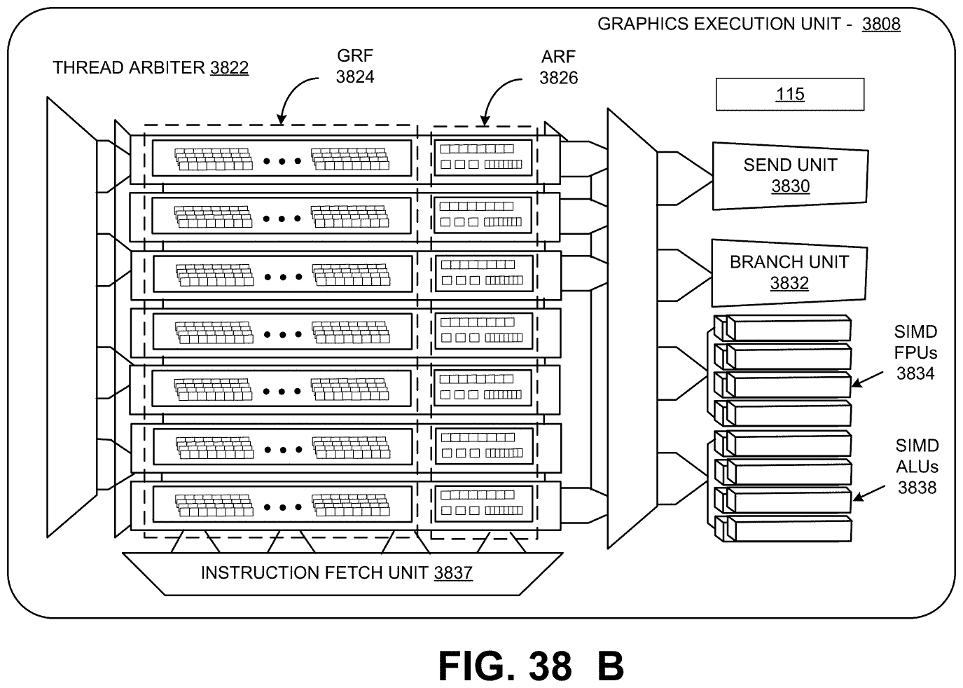

FIGS. 38A-38B illustrate thread execution logic 3800 including an array of processing elements of a graphics processor core according to at least one embodiment. FIG. 38A illustrates at least one embodiment, in which thread execution logic 3800 is used. FIG. 38B illustrates exemplary internal details of a graphics execution unit 3808, according to at least one embodiment.

As illustrated in FIG. 38A, in at least one embodiment, thread execution logic 3800 includes a shader processor 3802, a thread dispatcher 3804, an instruction cache 3806, a scalable execution unit array including a plurality of execution units 3807A-3807N and 3808A-3808N, a sampler 3810, a data cache 3812, and a data port 3814. In at least one embodiment, a scalable execution unit array can dynamically scale by enabling or disabling one or more execution units (e.g., any of execution unit 3808A-N or 3807A-N) based on computational requirements of a workload, for example. In at least one embodiment, scalable execution units are interconnected via an interconnect fabric that links to each execution unit. In at least one embodiment, thread execution logic 3800 includes one or more connections to memory, such as system memory or cache memory, through one or more of instruction cache 3806, data port 3814, sampler 3810, and execution units 3807 or 3808. In at least one embodiment, each execution unit (e.g., 3807A) is a stand-alone programmable general-purpose computational unit that is capable of executing multiple simultaneous hardware threads while processing multiple data elements in parallel for each thread. In at least one embodiment, array of execution units 3807 and/or 3808 is scalable to include any number individual execution units.

In at least one embodiment, execution units 3807 and/or 3808 are primarily used to execute shader programs. In at least one embodiment, shader processor 3802 can process various shader programs and dispatch execution threads associated with shader programs via a thread dispatcher 3804. In at least one embodiment, thread dispatcher 3804 includes logic to arbitrate thread initiation requests from graphics and media pipelines and instantiate requested threads on one or more execution units in execution units 3807 and/or 3808. For example, in at least one embodiment, a geometry pipeline can dispatch vertex, tessellation, or geometry shaders to thread execution logic for processing. In at least one embodiment, thread dispatcher 3804 can also process runtime thread spawning requests from executing shader programs.

In at least one embodiment, execution units 3807 and/or 3808 support an instruction set that includes native support for many standard 3D graphics shader instructions, such that shader programs from graphics libraries (e.g., Direct 3D and OpenGL) are executed with a minimal translation. In at least one embodiment, execution units support vertex and geometry processing (e.g., vertex programs, geometry programs, and/or vertex shaders), pixel processing (e.g., pixel shaders, fragment shaders) and general-purpose processing (e.g., compute and media shaders). In at least one embodiment, each of execution units 3807 and/or 3808, which include one or more arithmetic logic units (ALUs), is capable of multi-issue single instruction multiple data (SIMD) execution and multi-threaded operation enables an efficient execution environment despite higher latency memory accesses. In at least one embodiment, each hardware thread within each execution unit has a dedicated high-bandwidth register file and associated independent thread-state. In at least one embodiment, execution is multi-issue per clock to pipelines capable of integer, single and double precision floating point operations, SIMD branch capability, logical operations, transcendental operations, and other miscellaneous operations. In at least one embodiment, while waiting for data from memory or one of shared functions, dependency logic within execution units 3807 and/or 3808 causes a waiting thread to sleep until requested data has been returned. In at least one embodiment, while an awaiting thread is sleeping, hardware resources may be devoted to processing other threads. For example, in at least one embodiment, during a delay associated with a vertex shader operation, an execution unit can perform operations for a pixel shader, fragment shader, or another type of shader program, including a different vertex shader.

In at least one embodiment, each execution unit in execution units 3807 and/or 3808 operates on arrays of data elements. In at least one embodiment, a number of data elements is an “execution size,” or number of channels for an instruction. In at least one embodiment, an execution channel is a logical unit of execution for data element access, masking, and flow control within instructions. In at least one embodiment, a number of channels may be independent of a number of physical arithmetic logic units (ALUs) or floating point units (FPUs) for a particular graphics processor. In at least one embodiment, execution units 3807 and/or 3808 support integer and floating-point data types.

In at least one embodiment, an execution unit instruction set includes SIMD instructions. In at least one embodiment, various data elements can be stored as a packed data type in a register and execution unit will process various elements based on data size of elements. For example, in at least one embodiment, when operating on a 256-bit wide vector, 256 bits of a vector are stored in a register and an execution unit operates on a vector as four separate 64-bit packed data elements (Quad-Word (QW) size data elements), eight separate 32-bit packed data elements (Double Word (DW) size data elements), sixteen separate 16-bit packed data elements (Word (W) size data elements), or thirty-two separate 8-bit data elements (byte (B) size data elements). However, in at least one embodiment, different vector widths and register sizes are possible.

In at least one embodiment, one or more execution units can be combined into a fused execution unit 3809A-3809N having thread control logic (3811A-3811N) that is common to fused EUs such as execution unit 3807A fused with execution unit 3808A into fused execution unit 3809A. In at least one embodiment, multiple EUs can be fused into an EU group. In at least one embodiment, each EU in a fused EU group can be configured to execute a separate SIMD hardware thread, with a number of EUs in a fused EU group possibly varying according to various embodiments. In at least one embodiment, various SIMD widths can be performed per-EU, including but not limited to SIMD8, SIMD16, and SIMD32. In at least one embodiment, each fused graphics execution unit 3809A-3809N includes at least two execution units. For example, in at least one embodiment, fused execution unit 3809A includes a first EU 3807A, second EU 3808A, and thread control logic 3811A that is common to first EU 3807A and second EU 3808A. In at least one embodiment, thread control logic 3811A controls threads executed on fused graphics execution unit 3809A, allowing each EU within fused execution units 3809A-3809N to execute using a common instruction pointer register.

In at least one embodiment, one or more internal instruction caches (e.g., 3806) are included in thread execution logic 3800 to cache thread instructions for execution units. In at least one embodiment, one or more data caches (e.g., 3812) are included to cache thread data during thread execution. In at least one embodiment, sampler 3810 is included to provide texture sampling for 3D operations and media sampling for media operations. In at least one embodiment, sampler 3810 includes specialized texture or media sampling functionality to process texture or media data during sampling process before providing sampled data to an execution unit.

During execution, in at least one embodiment, graphics and media pipelines send thread initiation requests to thread execution logic 3800 via thread spawning and dispatch logic. In at least one embodiment, once a group of geometric objects has been processed and rasterized into pixel data, pixel processor logic (e.g., pixel shader logic, fragment shader logic, etc.) within shader processor 3802 is invoked to further compute output information and cause results to be written to output surfaces (e.g., color buffers, depth buffers, stencil buffers, etc.). In at least one embodiment, a pixel shader or a fragment shader calculates values of various vertex attributes that are to be interpolated across a rasterized object. In at least one embodiment, pixel processor logic within shader processor 3802 then executes an application programming interface (API)-supplied pixel or fragment shader program. In at least one embodiment, to execute a shader program, shader processor 3802 dispatches threads to an execution unit (e.g., 3808A) via thread dispatcher 3804. In at least one embodiment, shader processor 3802 uses texture sampling logic in sampler 3810 to access texture data in texture maps stored in memory. In at least one embodiment, arithmetic operations on texture data and input geometry data compute pixel color data for each geometric fragment, or discards one or more pixels from further processing.

In at least one embodiment, data port 3814 provides a memory access mechanism for thread execution logic 3800 to output processed data to memory for further processing on a graphics processor output pipeline. In at least one embodiment, data port 3814 includes or couples to one or more cache memories (e.g., data cache 3812) to cache data for memory access via a data port.

As illustrated in FIG. 38B, in at least one embodiment, a graphics execution unit 3808 can include an instruction fetch unit 3838, a general register file array (GRF) 3824, an architectural register file array (ARF) 3826, a thread arbiter 3822, a send unit 3830, a branch unit 3832, a set of SIMD floating point units (FPUs) 3834, and a set of dedicated integer SIMD ALUs 3835. In at least one embodiment, GRF 3824 and ARF 3826 includes a set of general register files and architecture register files associated with each simultaneous hardware thread that may be active in graphics execution unit 3808. In at least one embodiment, per thread architectural state is maintained in ARF 3826, while data used during thread execution is stored in GRF 3824. In at least one embodiment, execution state of each thread, including instruction pointers for each thread, can be held in thread-specific registers in ARF 3826.

In at least one embodiment, graphics execution unit 3808 has an architecture that is a combination of Simultaneous Multi-Threading (SMT) and fine-grained Interleaved Multi-Threading (IMT). In at least one embodiment, architecture has a modular configuration that can be fine-tuned at design time based on a target number of simultaneous threads and number of registers per execution unit, where execution unit resources are divided across logic used to execute multiple simultaneous threads.

In at least one embodiment, graphics execution unit 3808 can co-issue multiple instructions, which may each be different instructions. In at least one embodiment, thread arbiter 3822 of graphics execution unit thread 3808 can dispatch instructions to one of send unit 3830, branch unit 3832, or SIMD FPU(s) 3834 for execution. In at least one embodiment, each execution thread can access 128 general-purpose registers within GRF 3824, where each register can store 32 bytes, accessible as a SIMD 8-element vector of 32-bit data elements. In at least one embodiment, each execution unit thread has access to 4 kilobytes within GRF 3824, although embodiments are not so limited, and greater or fewer register resources may be provided in other embodiments. In at least one embodiment, up to seven threads can execute simultaneously, although a number of threads per execution unit can also vary according to embodiments. In at least one embodiment, in which seven threads may access 4 kilobytes, GRF 3824 can store a total of 28 kilobytes. In at least one embodiment, flexible addressing modes can permit registers to be addressed together to build effectively wider registers or to represent strided rectangular block data structures.

In at least one embodiment, memory operations, sampler operations, and other longer-latency system communications are dispatched via “send” instructions that are executed by message passing to send unit 3830. In at least one embodiment, branch instructions are dispatched to branch unit 3832 to facilitate SIMD divergence and eventual convergence.

In at least one embodiment, graphics execution unit 3808 includes one or more SIMD floating point units (FPU(s)) 3834 to perform floating-point operations. In at least one embodiment, FPU(s) 3834 also support integer computation. In at least one embodiment, FPU(s) 3834 can SIMD execute up to M number of 32-bit floating-point (or integer) operations, or SIMD execute up to 2M 16-bit integer or 16-bit floating-point operations. In at least one embodiment, at least one FPU provides extended math capability to support high-throughput transcendental math functions and double precision 64-bit floating-point. In at least one embodiment, a set of 8-bit integer SIMD ALUs 3835 are also present, and may be specifically optimized to perform operations associated with machine learning computations.

In at least one embodiment, arrays of multiple instances of graphics execution unit 3808 can be instantiated in a graphics sub-core grouping (e.g., a sub-slice). In at least one embodiment, execution unit 3808 can execute instructions across a plurality of execution channels. In at least one embodiment, each thread executed on graphics execution unit 3808 is executed on a different channel.

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, portions or all of inference and/or training logic 115 may be incorporated into thread execution logic 3800. Moreover, in at least one embodiment, inferencing and/or training operations described herein may be done using logic other than logic illustrated in FIG. 1A or 1B. In at least one embodiment, weight parameters may be stored in on-chip or off-chip memory and/or registers (shown or not shown) that configure ALUs thread of execution logic 3800 to perform one or more machine learning algorithms, neural network architectures, use cases, or training techniques described herein.

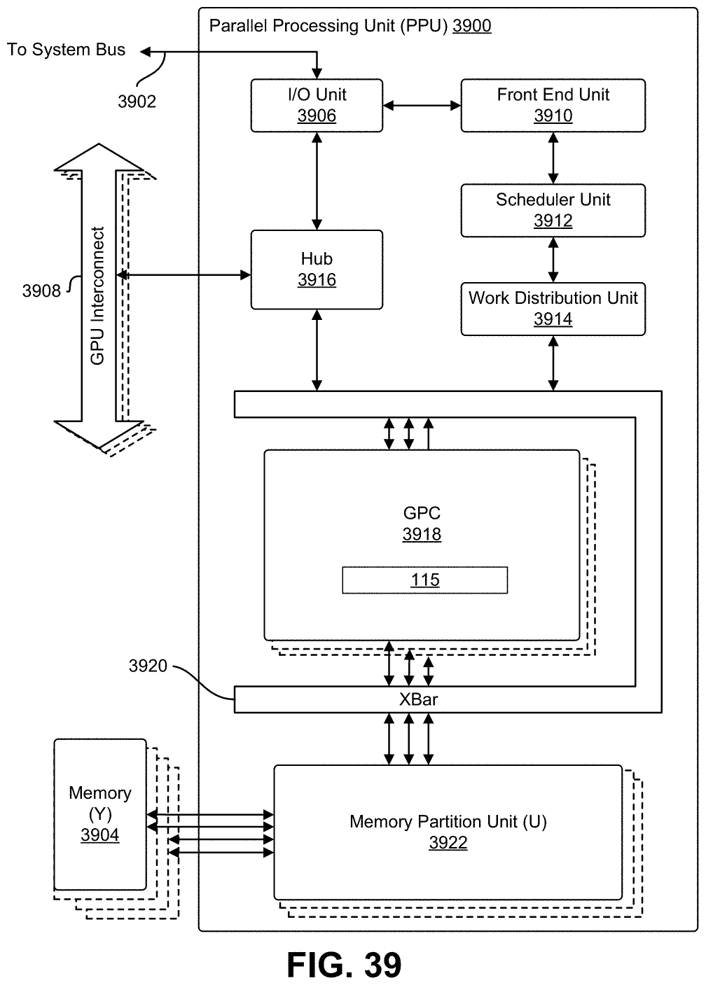

FIG. 39 illustrates a parallel processing unit (“PPU”) 3900, according to at least one embodiment. In at least one embodiment, PPU 3900 is configured with machine-readable code that, if executed by PPU 3900, causes PPU 3900 to perform some or all of processes and techniques described throughout this disclosure. In at least one embodiment, PPU 3900 is a multi-threaded processor that is implemented on one or more integrated circuit devices and that utilizes multithreading as a latency-hiding technique designed to process computer-readable instructions (also referred to as machine-readable instructions or simply instructions) on multiple threads in parallel. In at least one embodiment, a thread refers to a thread of execution and is an instantiation of a set of instructions configured to be executed by PPU 3900. In at least one embodiment, PPU 3900 is a graphics processing unit (“GPU”) configured to implement a graphics rendering pipeline for processing three-dimensional (“3D”) graphics data in order to generate two-dimensional (“2D”) image data for display on a display device such as a liquid crystal display (“LCD”) device. In at least one embodiment, PPU 3900 is utilized to perform computations such as linear algebra operations and machine-learning operations. FIG. 39 illustrates an example parallel processor for illustrative purposes only and should be construed as a non-limiting example of processor architectures contemplated within scope of this disclosure and that any suitable processor may be employed to supplement and/or substitute for same.

In at least one embodiment, one or more PPUs 3900 are configured to accelerate High Performance Computing (“HPC”), data center, and machine learning applications. In at least one embodiment, PPU 3900 is configured to accelerate deep learning systems and applications including following non-limiting examples: autonomous vehicle platforms, deep learning, high-accuracy speech, image, text recognition systems, intelligent video analytics, molecular simulations, drug discovery, disease diagnosis, weather forecasting, big data analytics, astronomy, molecular dynamics simulation, financial modeling, robotics, factory automation, real-time language translation, online search optimizations, and personalized user recommendations, and more.

In at least one embodiment, PPU 3900 includes, without limitation, an Input/Output (“I/O”) unit 3906, a front-end unit 3910, a scheduler unit 3912, a work distribution unit 3914, a hub 3916, a crossbar (“XBar”) 3920, one or more general processing clusters (“GPCs”) 3918, and one or more partition units (“memory partition units”) 3922. In at least one embodiment, PPU 3900 is connected to a host processor or other PPUs 3900 via one or more high-speed GPU interconnects (“GPU interconnects”) 3908. In at least one embodiment, PPU 3900 is connected to a host processor or other peripheral devices via a system bus 3902. In at least one embodiment, PPU 3900 is connected to a local memory comprising one or more memory devices (“memory”) 3904. In at least one embodiment, memory devices 3904 include, without limitation, one or more dynamic random access memory (“DRAM”) devices. In at least one embodiment, one or more DRAM devices are configured and/or configurable as high-bandwidth memory (“HBM”) subsystems, with multiple DRAM dies stacked within each device.

In at least one embodiment, high-speed GPU interconnect 3908 may refer to a wire-based multi-lane communications link that is used by systems to scale and include one or more PPUs 3900 combined with one or more central processing units (“CPUs”), supports cache coherence between PPUs 3900 and CPUs, and CPU mastering. In at least one embodiment, data and/or commands are transmitted by high-speed GPU interconnect 3908 through hub 3916 to/from other units of PPU 3900 such as one or more copy engines, video encoders, video decoders, power management units, and other components which may not be explicitly illustrated in FIG. 39.

In at least one embodiment, I/O unit 3906 is configured to transmit and receive communications (e.g., commands, data) from a host processor (not illustrated in FIG. 38) over system bus 3802. In at least one embodiment, I/O unit 3806 communicates with host processor directly via system bus 3802 or through one or more intermediate devices such as a memory bridge. In at least one embodiment, I/O unit 3806 may communicate with one or more other processors, such as one or more of PPUs 3800 via system bus 3802. In at least one embodiment, I/O unit 3806 implements a Peripheral Component Interconnect Express (“PCIe”) interface for communications over a PCIe bus. In at least one embodiment, I/O unit 3806 implements interfaces for communicating with external devices.

In at least one embodiment, I/O unit 3906 decodes packets received via system bus 3902. In at least one embodiment, at least some packets represent commands configured to cause PPU 3900 to perform various operations. In at least one embodiment, I/O unit 3906 transmits decoded commands to various other units of PPU 3900 as specified by commands. In at least one embodiment, commands are transmitted to front-end unit 3910 and/or transmitted to hub 3916 or other units of PPU 3900 such as one or more copy engines, a video encoder, a video decoder, a power management unit, etc. (not explicitly illustrated in FIG. 39). In at least one embodiment, I/O unit 3906 is configured to route communications between and among various logical units of PPU 3900.

In at least one embodiment, a program executed by host processor encodes a command stream in a buffer that provides workloads to PPU 3900 for processing. In at least one embodiment, a workload comprises instructions and data to be processed by those instructions. In at least one embodiment, a buffer is a region in a memory that is accessible (e.g., read/write) by both a host processor and PPU 3900—a host interface unit may be configured to access that buffer in a system memory connected to system bus 3902 via memory requests transmitted over system bus 3902 by I/O unit 3906. In at least one embodiment, a host processor writes a command stream to a buffer and then transmits a pointer to a start of a command stream to PPU 3900 such that front-end unit 3910 receives pointers to one or more command streams and manages one or more command streams, reading commands from command streams and forwarding commands to various units of PPU 3900.

In at least one embodiment, front-end unit 3910 is coupled to scheduler unit 3912 that configures various GPCs 3918 to process tasks defined by one or more command streams. In at least one embodiment, scheduler unit 3912 is configured to track state information related to various tasks managed by scheduler unit 3912 where state information may indicate which of GPCs 3918 a task is assigned to, whether task is active or inactive, a priority level associated with task, and so forth. In at least one embodiment, scheduler unit 3912 manages execution of a plurality of tasks on one or more of GPCs 3918.

In at least one embodiment, scheduler unit 3912 is coupled to work distribution unit 3914 that is configured to dispatch tasks for execution on GPCs 3918. In at least one embodiment, work distribution unit 3914 tracks a number of scheduled tasks received from scheduler unit 3912 and work distribution unit 3914 manages a pending task pool and an active task pool for each of GPCs 3918. In at least one embodiment, pending task pool comprises a number of slots (e.g., 32 slots) that contain tasks assigned to be processed by a particular GPC 3918; an active task pool may comprise a number of slots (e.g., 4 slots) for tasks that are actively being processed by GPCs 3918 such that as one of GPCs 3918 completes execution of a task, that task is evicted from that active task pool for GPC 3918 and another task from a pending task pool is selected and scheduled for execution on GPC 3918. In at least one embodiment, if an active task is idle on GPC 3918, such as while waiting for a data dependency to be resolved, then that active task is evicted from GPC 3918 and returned to that pending task pool while another task in that pending task pool is selected and scheduled for execution on GPC 3918.

In at least one embodiment, work distribution unit 3914 communicates with one or more GPCs 3918 via XBar 3920. In at least one embodiment, XBar 3920 is an interconnect network that couples many of units of PPU 3900 to other units of PPU 3900 and can be configured to couple work distribution unit 3914 to a particular GPC 3918. In at least one embodiment, one or more other units of PPU 3900 may also be connected to XBar 3920 via hub 3916.

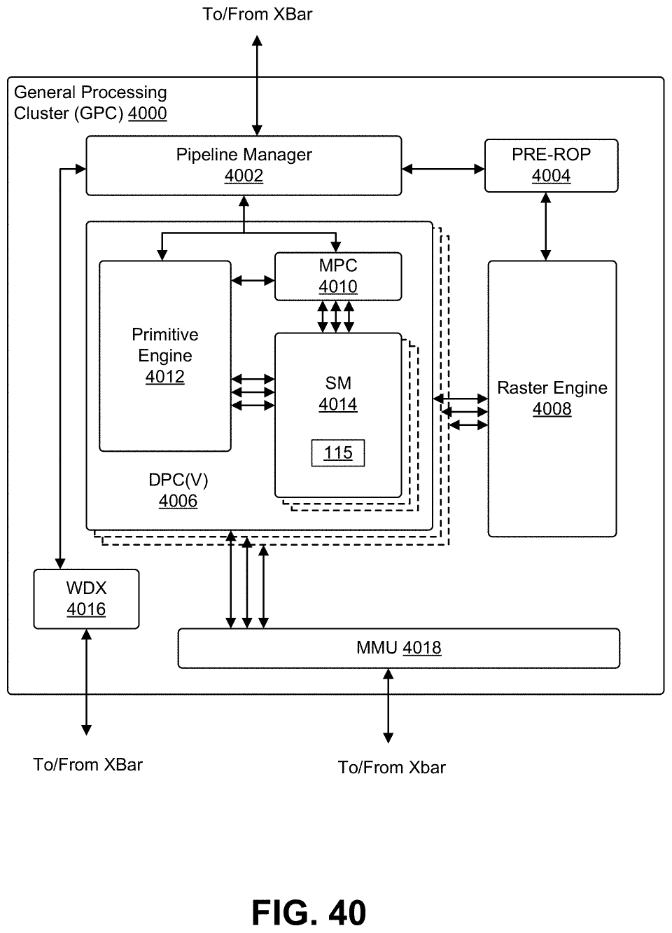

In at least one embodiment, tasks are managed by scheduler unit 3912 and dispatched to one of GPCs 3918 by work distribution unit 3914. In at least one embodiment, GPC 3918 is configured to process task and generate results. In at least one embodiment, results may be consumed by other tasks within GPC 3918, routed to a different GPC 3918 via XBar 3920, or stored in memory 3904. In at least one embodiment, results can be written to memory 3904 via partition units 3922, which implement a memory interface for reading and writing data to/from memory 3904. In at least one embodiment, results can be transmitted to another PPU 3900 or CPU via high-speed GPU interconnect 3908. In at least one embodiment, PPU 3900 includes, without limitation, a number U of partition units 3922 that is equal to a number of separate and distinct memory devices 3904 coupled to PPU 3900, as described in more detail herein in conjunction with FIG. 40.

In at least one embodiment, a host processor executes a driver kernel that implements an application programming interface (“API”) that enables one or more applications executing on a host processor to schedule operations for execution on PPU 3900. In at least one embodiment, multiple compute applications are simultaneously executed by PPU 3900 and PPU 3900 provides isolation, quality of service (“QoS”), and independent address spaces for multiple compute applications. In at least one embodiment, an application generates instructions (e.g., in form of API calls) that cause a driver kernel to generate one or more tasks for execution by PPU 3900 and that driver kernel outputs tasks to one or more streams being processed by PPU 3900. In at least one embodiment, each task comprises one or more groups of related threads, which may be referred to as a warp. In at least one embodiment, a warp comprises a plurality of related threads (e.g., 32 threads) that can be executed in parallel. In at least one embodiment, cooperating threads can refer to a plurality of threads including instructions to perform task and that exchange data through shared memory. In at least one embodiment, threads and cooperating threads are described in more detail in conjunction with FIG. 40.

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, deep learning application processor is used to train a machine learning model, such as a neural network, to predict or infer information provided to PPU 3900. In at least one embodiment, PPU 3900 is used to infer or predict information based on a trained machine learning model (e.g., neural network) that has been trained by another processor or system or by PPU 3900. In at least one embodiment, PPU 3900 may be used to perform one or more neural network use cases described herein.

FIG. 40 illustrates a general processing cluster (“GPC”) 4000, according to at least one embodiment. In at least one embodiment, GPC 4000 is GPC 3918 of FIG. 39. In at least one embodiment, each GPC 4000 includes, without limitation, a number of hardware units for processing tasks and each GPC 4000 includes, without limitation, a pipeline manager 4002, a pre-raster operations unit (“preROP”) 4004, a raster engine 4008, a work distribution crossbar (“WDX”) 4016, a memory management unit (“MMU”) 4018, one or more Data Processing Clusters (“DPCs”) 4006, and any suitable combination of parts.

In at least one embodiment, operation of GPC 4000 is controlled by pipeline manager 4002. In at least one embodiment, pipeline manager 4002 manages configuration of one or more DPCs 4006 for processing tasks allocated to GPC 4000. In at least one embodiment, pipeline manager 4002 configures at least one of one or more DPCs 4006 to implement at least a portion of a graphics rendering pipeline. In at least one embodiment, DPC 4006 is configured to execute a vertex shader program on a programmable streaming multi-processor (“SM”) 4014. In at least one embodiment, pipeline manager 4002 is configured to route packets received from a work distribution unit to appropriate logical units within GPC 4000, in at least one embodiment, and some packets may be routed to fixed function hardware units in preROP 4004 and/or raster engine 4008 while other packets may be routed to DPCs 4006 for processing by a primitive engine 4012 or SM 4014. In at least one embodiment, pipeline manager 4002 configures at least one of DPCs 4006 to implement a neural network model and/or a computing pipeline.

In at least one embodiment, preROP unit 4004 is configured, in at least one embodiment, to route data generated by raster engine 4008 and DPCs 4006 to a Raster Operations (“ROP”) unit in partition unit 3822, described in more detail above in conjunction with FIG. 38. In at least one embodiment, preROP unit 4004 is configured to perform optimizations for color blending, organize pixel data, perform address translations, and more. In at least one embodiment, raster engine 4008 includes, without limitation, a number of fixed function hardware units configured to perform various raster operations, in at least one embodiment, and raster engine 4008 includes, without limitation, a setup engine, a coarse raster engine, a culling engine, a clipping engine, a fine raster engine, a tile coalescing engine, and any suitable combination thereof. In at least one embodiment, setup engine receives transformed vertices and generates plane equations associated with geometric primitive defined by vertices; plane equations are transmitted to a coarse raster engine to generate coverage information (e.g., an x, y coverage mask for a tile) for primitive; output of a coarse raster engine is transmitted to a culling engine where fragments associated with a primitive that fail a z-test are culled, and transmitted to a clipping engine where fragments lying outside a viewing frustum are clipped. In at least one embodiment, fragments that survive clipping and culling are passed to a fine raster engine to generate attributes for pixel fragments based on plane equations generated by a setup engine. In at least one embodiment, an output of raster engine 4008 comprises fragments to be processed by any suitable entity, such as by a fragment shader implemented within DPC 4006.

In at least one embodiment, each DPC 4006 included in GPC 4000 comprises, without limitation, an M-Pipe Controller (“MPC”) 4010; primitive engine 4012; one or more SMs 4014; and any suitable combination thereof. In at least one embodiment, MPC 4010 controls operation of DPC 4006, routing packets received from pipeline manager 4002 to appropriate units in DPC 4006. In at least one embodiment, packets associated with a vertex are routed to primitive engine 4012, which is configured to fetch vertex attributes associated with a vertex from memory; in contrast, packets associated with a shader program may be transmitted to SM 4014.

In at least one embodiment, SM 4014 comprises, without limitation, a programmable streaming processor that is configured to process tasks represented by a number of threads. In at least one embodiment, SM 4014 is multi-threaded and configured to execute a plurality of threads (e.g., 32 threads) from a particular group of threads concurrently and implements a Single-Instruction, Multiple-Data (“SIMD”) architecture where each thread in a group of threads (e.g., a warp) is configured to process a different set of data based on same set of instructions. In at least one embodiment, all threads in group of threads execute a common set of instructions. In at least one embodiment, SM 4014 implements a Single-Instruction, Multiple Thread (“SIMT”) architecture wherein each thread in a group of threads is configured to process a different set of data based on that common set of instructions, but where individual threads in a group of threads are allowed to diverge during execution. In at least one embodiment, a program counter, call stack, and execution state is maintained for each warp, enabling concurrency between warps and serial execution within warps when threads within a warp diverge. In at least one embodiment, a program counter, call stack, and execution state is maintained for each individual thread, enabling equal concurrency between all threads, within and between warps. In at least one embodiment, execution state is maintained for each individual thread and threads executing common instructions may be converged and executed in parallel for better efficiency. At least one embodiment of SM 4014 is described in more detail herein.

In at least one embodiment, MMU 4018 provides an interface between GPC 4000 and a memory partition unit (e.g., partition unit 3922 of FIG. 39) and MMU 4018 provides translation of virtual addresses into physical addresses, memory protection, and arbitration of memory requests. In at least one embodiment, MMU 4018 provides one or more translation lookaside buffers (“TLBs”) for performing translation of virtual addresses into physical addresses in memory.

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, deep learning application processor is used to train a machine learning model, such as a neural network, to predict or infer information provided to GPC 4000. In at least one embodiment, GPC 4000 is used to infer or predict information based on a trained machine learning model (e.g., neural network) that has been trained by another processor or system or by GPC 4000. In at least one embodiment, GPC 4000 may be used to perform one or more neural network use cases described herein.

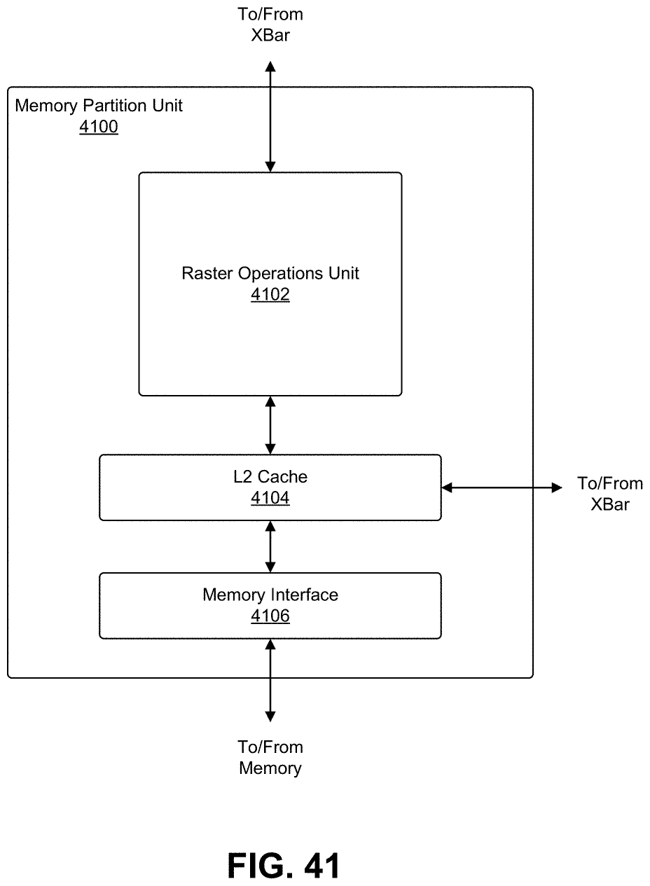

FIG. 41 illustrates a memory partition unit 4100 of a parallel processing unit (“PPU”), in accordance with at least one embodiment. In at least one embodiment, memory partition unit 4100 includes, without limitation, a Raster Operations (“ROP”) unit 4102, a level two (“L2”) cache 4104, a memory interface 4106, and any suitable combination thereof. In at least one embodiment, memory interface 4106 is coupled to memory. In at least one embodiment, memory interface 4106 may implement 32, 64, 96, 1024-bit data buses, or like, for high-speed data transfer. In at least one embodiment, PPU incorporates U memory interfaces 4106 where U is a positive integer, with one memory interface 4106 per pair of partition units 4100, where each pair of partition units 4100 is connected to a corresponding memory device. For example, in at least one embodiment, PPU may be connected to up to Y memory devices, such as high bandwidth memory stacks or graphics double-data-rate, version 5, synchronous dynamic random access memory (“GDDR5 SDRAM”).

In at least one embodiment, memory interface 4106 implements a high bandwidth memory second generation (“HBM2”) memory interface and Y equals half of U. In at least one embodiment, HBM2 memory stacks are located on a physical package with a PPU, providing substantial power and area savings compared with conventional GDDR5 SDRAM systems. In at least one embodiment, each HBM2 stack includes, without limitation, four memory dies with Y=4, with each HBM2 stack including two 128-bit channels per die for a total of 8 channels and a data bus width of 1024 bits. In at least one embodiment, that memory supports Single-Error Correcting Double-Error Detecting (“SECDED”) Error Correction Code (“ECC”) to protect data. In at least one embodiment, ECC can provide higher reliability for compute applications that are sensitive to data corruption.

In at least one embodiment, PPU implements a multi-level memory hierarchy. In at least one embodiment, memory partition unit 4100 supports a unified memory to provide a single unified virtual address space for central processing unit (“CPU”) and PPU memory, enabling data sharing between virtual memory systems. In at least one embodiment frequency of accesses by a PPU to a memory located on other processors is traced to ensure that memory pages are moved to physical memory of PPU that is accessing pages more frequently. In at least one embodiment, high-speed GPU interconnect 3808 supports address translation services allowing PPU to directly access a CPU's page tables and providing full access to CPU memory by a PPU.

In at least one embodiment, copy engines transfer data between multiple PPUs or between PPUs and CPUs. In at least one embodiment, copy engines can generate page faults for addresses that are not mapped into page tables and memory partition unit 4100 then services page faults, mapping addresses into page table, after which copy engine performs a transfer. In at least one embodiment, memory is pinned (i.e., non-pageable) for multiple copy engine operations between multiple processors, substantially reducing available memory. In at least one embodiment, with hardware page faulting, addresses can be passed to copy engines without regard as to whether memory pages are resident, and a copy process is transparent.

Data from memory 3904 of FIG. 39 or other system memory is fetched by memory partition unit 4100 and stored in L2 cache 4104, which is located on-chip and is shared between various GPCs, in accordance with at least one embodiment. Each memory partition unit 4100, in at least one embodiment, includes, without limitation, at least a portion of L2 cache associated with a corresponding memory device. In at least one embodiment, lower level caches are implemented in various units within GPCs. In at least one embodiment, each of SMs 4014 in FIG. 40 may implement a Level 1 (“L1”) cache wherein that L1 cache is private memory that is dedicated to a particular SM 4014 and data from L2 cache 4104 is fetched and stored in each L1 cache for processing in functional units of SMs 4014. In at least one embodiment, L2 cache 4104 is coupled to memory interface 4106 and XBar 3920 shown in FIG. 39.

In at least one embodiment, ROP unit 4102 performs graphics raster operations related to pixel color, such as color compression, pixel blending, and more, in at least one embodiment. ROP unit 4102, in at least one embodiment, implements depth testing in conjunction with raster engine 4008, receiving a depth for a sample location associated with a pixel fragment from a culling engine of raster engine 4008. In at least one embodiment, depth is tested against a corresponding depth in a depth buffer for a sample location associated with a fragment. In at least one embodiment, if that fragment passes that depth test for that sample location, then ROP unit 4102 updates depth buffer and transmits a result of that depth test to raster engine 4008. It will be appreciated that a number of partition units 4100 may be different than a number of GPCs and, therefore, each ROP unit 4102 can, in at least one embodiment, be coupled to each GPC. In at least one embodiment, ROP unit 4102 tracks packets received from different GPCs and determines whether a result generated by ROP unit 4102 is to be routed to through XBar 3820.

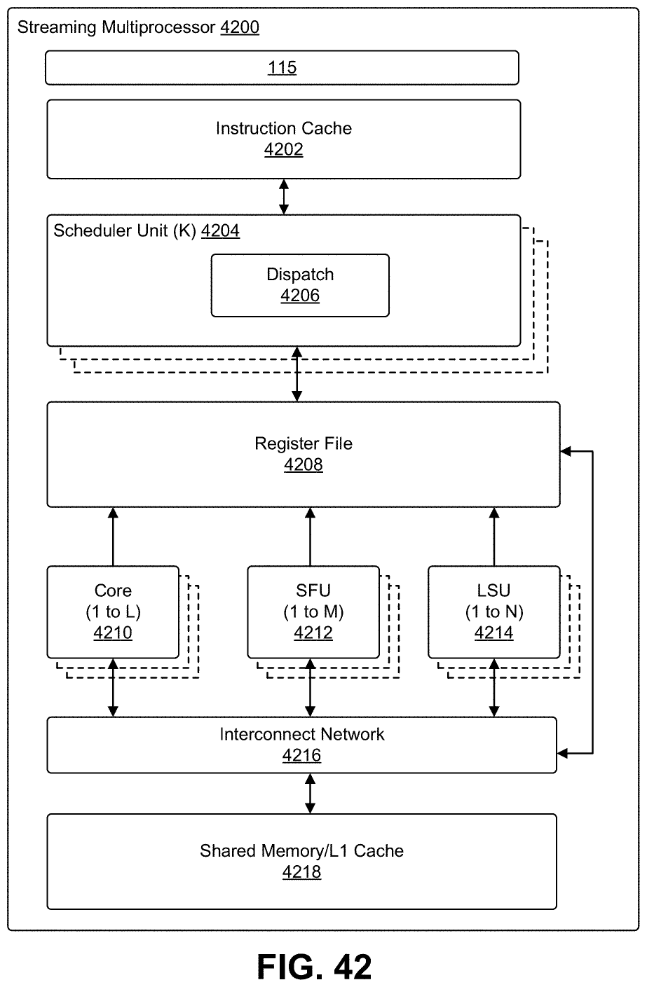

FIG. 42 illustrates a streaming multi-processor (“SM”) 4200, according to at least one embodiment. In at least one embodiment, SM 4200 is SM 4014 of FIG. 40. In at least one embodiment, SM 4200 includes, without limitation, an instruction cache 4202, one or more scheduler units 4204, a register file 4208, one or more processing cores (“cores”) 4210, one or more special function units (“SFUs”) 4212, one or more load/store units (“LSUs”) 4214, an interconnect network 4216, a shared memory/level one (“L1”) cache 4218, and/or any suitable combination thereof.

In at least one embodiment, a work distribution unit dispatches tasks for execution on general processing clusters (“GPCs”) of parallel processing units (“PPUs”) and each task is allocated to a particular Data Processing Cluster (“DPC”) within a GPC and, if a task is associated with a shader program, that task is allocated to one of SMs 4200. In at least one embodiment, scheduler unit 4204 receives tasks from a work distribution unit and manages instruction scheduling for one or more thread blocks assigned to SM 4200. In at least one embodiment, scheduler unit 4204 schedules thread blocks for execution as warps of parallel threads, wherein each thread block is allocated at least one warp. In at least one embodiment, each warp executes threads. In at least one embodiment, scheduler unit 4204 manages a plurality of different thread blocks, allocating warps to different thread blocks and then dispatching instructions from plurality of different cooperative groups to various functional units (e.g., processing cores 4210, SFUs 4212, and LSUs 4214) during each clock cycle.

In at least one embodiment, Cooperative Groups may refer to a programming model for organizing groups of communicating threads that allows developers to express granularity at which threads are communicating, enabling expression of richer, more efficient parallel decompositions. In at least one embodiment, cooperative launch APIs support synchronization amongst thread blocks for execution of parallel algorithms. In at least one embodiment, applications of conventional programming models provide a single, simple construct for synchronizing cooperating threads: a barrier across all threads of a thread block (e.g., syncthreads( ) function). However, in at least one embodiment, programmers may define groups of threads at smaller than thread block granularities and synchronize within defined groups to enable greater performance, design flexibility, and software reuse in form of collective group-wide function interfaces. In at least one embodiment, Cooperative Groups enables programmers to define groups of threads explicitly at sub-block (i.e., as small as a single thread) and multi-block granularities, and to perform collective operations such as synchronization on threads in a cooperative group. In at least one embodiment, that programming model supports clean composition across software boundaries, so that libraries and utility functions can synchronize safely within their local context without having to make assumptions about convergence. In at least one embodiment, Cooperative Groups primitives enable new patterns of cooperative parallelism, including, without limitation, producer-consumer parallelism, opportunistic parallelism, and global synchronization across an entire grid of thread blocks.

In at least one embodiment, a dispatch unit 4206 is configured to transmit instructions to one or more functional units and scheduler unit 4204 and includes, without limitation, two dispatch units 4206 that enable two different instructions from a common warp to be dispatched during each clock cycle. In at least one embodiment, each scheduler unit 4204 includes a single dispatch unit 4206 or additional dispatch units 4206.

In at least one embodiment, each SM 4200, in at least one embodiment, includes, without limitation, register file 4208 that provides a set of registers for functional units of SM 4200. In at least one embodiment, register file 4208 is divided between each functional unit such that each functional unit is allocated a dedicated portion of register file 4208. In at least one embodiment, register file 4208 is divided between different warps being executed by SM 4200 and register file 4208 provides temporary storage for operands connected to data paths of functional units. In at least one embodiment, each SM 4200 comprises, without limitation, a plurality of L processing cores 4210, where L is a positive integer. In at least one embodiment, SM 4200 includes, without limitation, a large number (e.g., 128 or more) of distinct processing cores 4210. In at least one embodiment, each processing core 4210 includes, without limitation, a fully-pipelined, single-precision, double-precision, and/or mixed precision processing unit that includes, without limitation, a floating point arithmetic logic unit and an integer arithmetic logic unit. In at least one embodiment, floating point arithmetic logic units implement IEEE 754-2008 standard for floating point arithmetic. In at least one embodiment, processing cores 4210 include, without limitation, 64 single-precision (32-bit) floating point cores, 64 integer cores, 32 double-precision (64-bit) floating point cores, and 8 tensor cores.

Tensor cores are configured to perform matrix operations in accordance with at least one embodiment. In at least one embodiment, one or more tensor cores are included in processing cores 4210. In at least one embodiment, tensor cores are configured to perform deep learning matrix arithmetic, such as convolution operations for neural network training and inferencing. In at least one embodiment, each tensor core operates on a 4×4 matrix and performs a matrix multiply and accumulate operation, D=A×B+C, where A, B, C, and D are 4×4 matrices.

In at least one embodiment, matrix multiply inputs A and B are 16-bit floating point matrices and accumulation matrices C and D are 16-bit floating point or 32-bit floating point matrices. In at least one embodiment, tensor cores operate on 16-bit floating point input data with 32-bit floating point accumulation. In at least one embodiment, 16-bit floating point multiply uses 64 operations and results in a full precision product that is then accumulated using 32-bit floating point addition with other intermediate products for a 4×4×4 matrix multiply. Tensor cores are used to perform much larger two-dimensional or higher dimensional matrix operations, built up from these smaller elements, in at least one embodiment. In at least one embodiment, an API, such as a CUDA 9 C++ API, exposes specialized matrix load, matrix multiply and accumulate, and matrix store operations to efficiently use tensor cores from a CUDA-C++ program. In at least one embodiment, at a CUDA level, a warp-level interface assumes 16×16 size matrices spanning all 32 threads of warp.

In at least one embodiment, each SM 4200 comprises, without limitation, M SFUs 4212 that perform special functions (e.g., attribute evaluation, reciprocal square root, and like). In at least one embodiment, SFUs 4212 include, without limitation, a tree traversal unit configured to traverse a hierarchical tree data structure. In at least one embodiment, SFUs 4212 include, without limitation, a texture unit configured to perform texture map filtering operations. In at least one embodiment, texture units are configured to load texture maps (e.g., a 2D array of texels) from memory and sample texture maps to produce sampled texture values for use in shader programs executed by SM 4200. In at least one embodiment, texture maps are stored in shared memory/L1 cache 4218. In at least one embodiment, texture units implement texture operations such as filtering operations using mip-maps (e.g., texture maps of varying levels of detail), in accordance with at least one embodiment. In at least one embodiment, each SM 4200 includes, without limitation, two texture units.

Each SM 4200 comprises, without limitation, N LSUs 4214 that implement load and store operations between shared memory/L1 cache 4218 and register file 4208, in at least one embodiment. Interconnect network 4216 connects each functional unit to register file 4208 and LSU 4214 to register file 4208 and shared memory/L1 cache 4218 in at least one embodiment. In at least one embodiment, interconnect network 4216 is a crossbar that can be configured to connect any functional units to any registers in register file 4208 and connect LSUs 4214 to register file 4208 and memory locations in shared memory/L1 cache 4218.

In at least one embodiment, shared memory/L1 cache 4218 is an array of on-chip memory that allows for data storage and communication between SM 4200 and primitive engine and between threads in SM 4200, in at least one embodiment. In at least one embodiment, shared memory/L1 cache 4218 comprises, without limitation, 128 KB of storage capacity and is in a path from SM 4200 to a partition unit. In at least one embodiment, shared memory/L1 cache 4218, in at least one embodiment, is used to cache reads and writes. In at least one embodiment, one or more of shared memory/L1 cache 4218, L2 cache, and memory are backing stores.

Combining data cache and shared memory functionality into a single memory block provides improved performance for both types of memory accesses, in at least one embodiment. In at least one embodiment, capacity is used or is usable as a cache by programs that do not use shared memory, such as if shared memory is configured to use half of a capacity, and texture and load/store operations can use remaining capacity. Integration within shared memory/L1 cache 4218 enables shared memory/L1 cache 4218 to function as a high-throughput conduit for streaming data while simultaneously providing high-bandwidth and low-latency access to frequently reused data, in accordance with at least one embodiment. In at least one embodiment, when configured for general purpose parallel computation, a simpler configuration can be used compared with graphics processing. In at least one embodiment, fixed function graphics processing units are bypassed, creating a much simpler programming model. In a general purpose parallel computation configuration, a work distribution unit assigns and distributes blocks of threads directly to DPCs, in at least one embodiment. In at least one embodiment, threads in a block execute a common program, using a unique thread ID in calculation to ensure each thread generates unique results, using SM 4200 to execute program and perform calculations, shared memory/L1 cache 4218 to communicate between threads, and LSU 4214 to read and write global memory through shared memory/L1 cache 4218 and memory partition unit. In at least one embodiment, when configured for general purpose parallel computation, SM 4200 writes commands that scheduler unit 4204 can use to launch new work on DPCs.

In at least one embodiment, a PPU is included in or coupled to a desktop computer, a laptop computer, a tablet computer, servers, supercomputers, a smart-phone (e.g., a wireless, hand-held device), personal digital assistant (“PDA”), a digital camera, a vehicle, a head mounted display, a hand-held electronic device, and more. In at least one embodiment, a PPU is embodied on a single semiconductor substrate. In at least one embodiment, a PPU is included in a system-on-a-chip (“SoC”) along with one or more other devices such as additional PPUs, memory, a reduced instruction set computer (“RISC”) CPU, a memory management unit (“MMU”), a digital-to-analog converter (“DAC”), and like.

In at least one embodiment, a PPU may be included on a graphics card that includes one or more memory devices. In at least one embodiment, that graphics card may be configured to interface with a PCIe slot on a motherboard of a desktop computer. In at least one embodiment, that PPU may be an integrated graphics processing unit (“iGPU”) included in chipset of a motherboard.

Inference and/or training logic 115 are used to perform inferencing and/or training operations associated with one or more embodiments. Details regarding inference and/or training logic 115 are provided herein in conjunction with FIGS. 1A and/or 1B. In at least one embodiment, deep learning application processor is used to train a machine learning model, such as a neural network, to predict or infer information provided to SM 4200. In at least one embodiment, SM 4200 is used to infer or predict information based on a trained machine learning model (e.g., neural network) that has been trained by another processor or system or by SM 4200. In at least one embodiment, SM 4200 may be used to perform one or more neural network use cases described herein.

Embodiments are disclosed related a virtualized computing platform for advanced computing.

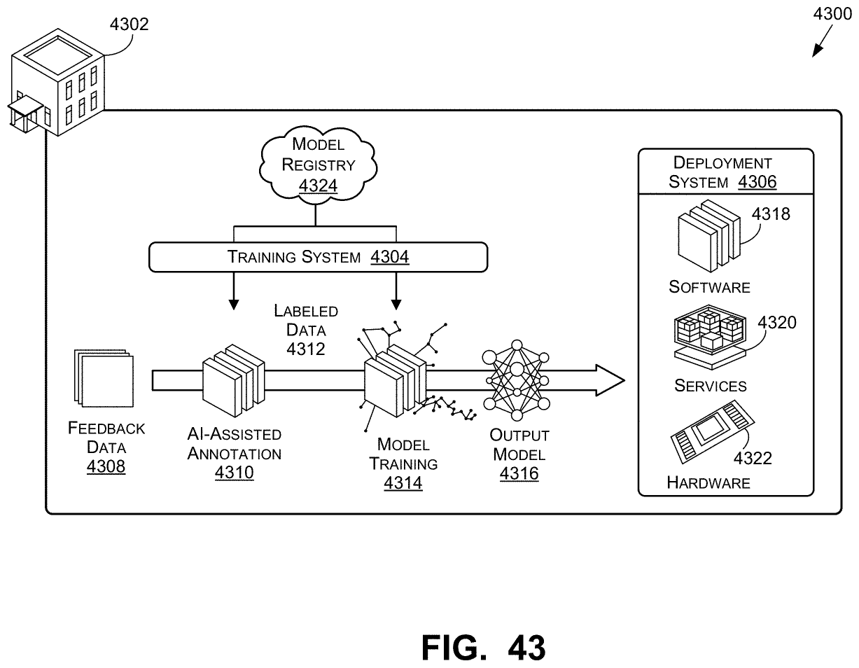

FIG. 43 is an example data flow diagram for a process 4300 of generating and deploying a processing and inferencing pipeline, in accordance with at least one embodiment. In at least one embodiment, process 4300 may be deployed to perform game name recognition analysis and inferencing on user feedback data at one or more facilities 4302, such as a data center.

In at least one embodiment, process 4300 may be executed within a training system 4304 and/or a deployment system 4306. In at least one embodiment, training system 4304 may be used to perform training, deployment, and implementation of machine learning models (e.g., neural networks, object detection algorithms, computer vision algorithms, etc.) for use in deployment system 4306. In at least one embodiment, deployment system 4306 may be configured to offload processing and compute resources among a distributed computing environment to reduce infrastructure requirements at facility 4302. In at least one embodiment, deployment system 4306 may provide a streamlined platform for selecting, customizing, and implementing virtual instruments for use with computing devices at facility 4302. In at least one embodiment, virtual instruments may include software-defined applications for performing one or more processing operations with respect to feedback data. In at least one embodiment, one or more applications in a pipeline may use or call upon services (e.g., inference, visualization, compute, AI, etc.) of deployment system 4306 during execution of applications.

In at least one embodiment, some of applications used in advanced processing and inferencing pipelines may use machine learning models or other AI to perform one or more processing steps. In at least one embodiment, machine learning models may be trained at facility 4302 using feedback data 4308 (such as feedback data) stored at facility 4302 or feedback data 4308 from another facility or facilities, or a combination thereof. In at least one embodiment, training system 4304 may be used to provide applications, services, and/or other resources for generating working, deployable machine learning models for deployment system 4306.

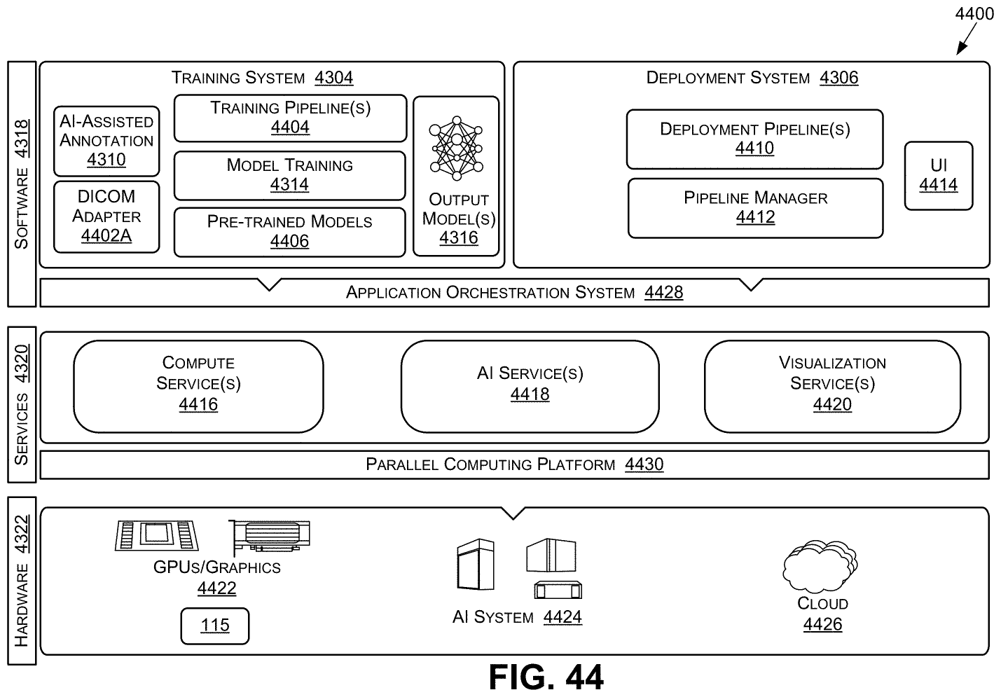

In at least one embodiment, a model registry 4324 may be backed by object storage that may support versioning and object metadata. In at least one embodiment, object storage may be accessible through, for example, a cloud storage (e.g., a cloud 4426 of FIG. 44) compatible application programming interface (API) from within a cloud platform. In at least one embodiment, machine learning models within model registry 4324 may uploaded, listed, modified, or deleted by developers or partners of a system interacting with an API. In at least one embodiment, an API may provide access to methods that allow users with appropriate credentials to associate models with applications, such that models may be executed as part of execution of containerized instantiations of applications.

In at least one embodiment, a training pipeline 4404 (FIG. 44) may include a scenario where facility 4302 is training their own machine learning model, or has an existing machine learning model that needs to be optimized or updated. In at least one embodiment, feedback data 4308 may be received from various channels, such as forums, web forms, or similar channels. In at least one embodiment, once feedback data 4308 is received, AI-assisted annotation 4310 may be used to aid in generating annotations corresponding to feedback data 4308 to be used as ground truth data for a machine learning model. In at least one embodiment, AI-assisted annotation 4310 may include one or more machine learning models (e.g., convolutional neural networks (CNNs)) that may be trained to generate annotations corresponding to certain types of feedback data 4308 (e.g., from certain devices) and/or certain types of anomalies in feedback data 4308. In at least one embodiment, AI-assisted annotations 4310 may then be used directly, or may be adjusted or fine-tuned using an annotation tool, to generate ground truth data. In at least one embodiment, in some examples, labeled data 4312 may be used as ground truth data for training a machine learning model. In at least one embodiment, AI-assisted annotations 4310, labeled data 4312, or a combination thereof may be used as ground truth data for training a machine learning model. In at least one embodiment, a trained machine learning model may be referred to as an output model 4316, and may be used by deployment system 4306, as described herein.

In at least one embodiment, training pipeline 4404 (FIG. 44) may include a scenario where facility 4302 needs a machine learning model for use in performing one or more processing tasks for one or more applications in deployment system 4306, but facility 4302 may not currently have such a machine learning model (or may not have a model that is optimized, efficient, or effective for such purposes). In at least one embodiment, an existing machine learning model may be selected from model registry 4324. In at least one embodiment, model registry 4324 may include machine learning models trained to perform a variety of different inference tasks on imaging data. In at least one embodiment, machine learning models in model registry 4324 may have been trained on imaging data from different facilities than facility 4302 (e.g., facilities remotely located). In at least one embodiment, machine learning models may have been trained on imaging data from one location, two locations, or any number of locations. In at least one embodiment, when being trained on imaging data from a specific location, training may take place at that location, or at least in a manner that protects confidentiality of imaging data or restricts imaging data from being transferred off-premises (e.g., to comply with HIPAA regulations, privacy regulations, etc.). In at least one embodiment, once a model is trained—or partially trained—at one location, a machine learning model may be added to model registry 4324. In at least one embodiment, a machine learning model may then be retrained, or updated, at any number of other facilities, and a retrained or updated model may be made available in model registry 4324. In at least one embodiment, a machine learning model may then be selected from model registry 4324—and referred to as output model 4316—and may be used in deployment system 4306 to perform one or more processing tasks for one or more applications of a deployment system.

In at least one embodiment, training pipeline 4304 (FIG. 43) may be used in a scenario that includes facility 4302 requiring a machine learning model for use in performing one or more processing tasks for one or more applications in deployment system 4306, but facility 4302 may not currently have such a machine learning model (or may not have a model that is optimized, efficient, or effective for such purposes). In at least one embodiment, a machine learning model selected from model registry 4324 might not be fine-tuned or optimized for feedback data 4308 generated at facility 4302 because of differences in populations, genetic variations, robustness of training data used to train a machine learning model, diversity in anomalies of training data, and/or other issues with training data. In at least one embodiment, AI-assisted annotation 4310 may be used to aid in generating annotations corresponding to feedback data 4308 to be used as ground truth data for retraining or updating a machine learning model. In at least one embodiment, labeled data 4312 may be used as ground truth data for training a machine learning model. In at least one embodiment, retraining or updating a machine learning model may be referred to as model training 4314. In at least one embodiment, model training 4314—e.g., AI-assisted annotations 4310, labeled data 4312, or a combination thereof—may be used as ground truth data for retraining or updating a machine learning model.

In at least one embodiment, deployment system 4306 may include software 4318, services 4320, hardware 4322, and/or other components, features, and functionality. In at least one embodiment, deployment system 4306 may include a software “stack,” such that software 4318 may be built on top of services 4320 and may use services 4320 to perform some or all of processing tasks, and services 4320 and software 4318 may be built on top of hardware 4322 and use hardware 4322 to execute processing, storage, and/or other compute tasks of deployment system 4306.

In at least one embodiment, software 4318 may include any number of different containers, where each container may execute an instantiation of an application. In at least one embodiment, each application may perform one or more processing tasks in an advanced processing and inferencing pipeline (e.g., inferencing, object detection, feature detection, segmentation, image enhancement, calibration, etc.). In at least one embodiment, for each type of computing device there may be any number of containers that may perform a data processing task with respect to feedback data 4308 (or other data types, such as those described herein). In at least one embodiment, an advanced processing and inferencing pipeline may be defined based on selections of different containers that are desired or required for processing feedback data 4308, in addition to containers that receive and configure imaging data for use by each container and/or for use by facility 4302 after processing through a pipeline (e.g., to convert outputs back to a usable data type for storage and display at facility 4302). In at least one embodiment, a combination of containers within software 4318 (e.g., that make up a pipeline) may be referred to as a virtual instrument (as described in more detail herein), and a virtual instrument may leverage services 4320 and hardware 4322 to execute some or all processing tasks of applications instantiated in containers.

In at least one embodiment, data may undergo pre-processing as part of data processing pipeline to prepare data for processing by one or more applications. In at least one embodiment, post-processing may be performed on an output of one or more inferencing tasks or other processing tasks of a pipeline to prepare an output data for a next application and/or to prepare output data for transmission and/or use by a user (e.g., as a response to an inference request). In at least one embodiment, inferencing tasks may be performed by one or more machine learning models, such as trained or deployed neural networks, which may include output models 4316 of training system 4304.

In at least one embodiment, tasks of data processing pipeline may be encapsulated in a container(s) that each represents a discrete, fully functional instantiation of an application and virtualized computing environment that is able to reference machine learning models. In at least one embodiment, containers or applications may be published into a private (e.g., limited access) area of a container registry (described in more detail herein), and trained or deployed models may be stored in model registry 4324 and associated with one or more applications. In at least one embodiment, images of applications (e.g., container images) may be available in a container registry, and once selected by a user from a container registry for deployment in a pipeline, an image may be used to generate a container for an instantiation of an application for use by a user's system.

In at least one embodiment, developers may develop, publish, and store applications (e.g., as containers) for performing processing and/or inferencing on supplied data. In at least one embodiment, development, publishing, and/or storing may be performed using a software development kit (SDK) associated with a system (e.g., to ensure that an application and/or container developed is compliant with or compatible with a system). In at least one embodiment, an application that is developed may be tested locally (e.g., at a first facility, on data from a first facility) with an SDK which may support at least some of services 4320 as a system (e.g., system 4400 of FIG. 44). In at least one embodiment, once validated by system 4400 (e.g., for accuracy, etc.), an application may be available in a container registry for selection and/or implementation by a user (e.g., a hospital, clinic, lab, healthcare provider, etc.) to perform one or more processing tasks with respect to data at a facility (e.g., a second facility) of a user.

In at least one embodiment, developers may then share applications or containers through a network for access and use by users of a system (e.g., system 4400 of FIG. 44). In at least one embodiment, completed and validated applications or containers may be stored in a container registry and associated machine learning models may be stored in model registry 4324. In at least one embodiment, a requesting entity—who provides an inference or image processing request—may browse a container registry and/or model registry 4324 for an application, container, dataset, machine learning model, etc., select a desired combination of elements for inclusion in data processing pipeline, and submit an processing request. In at least one embodiment, a request may include input data that is necessary to perform a request, and/or may include a selection of application(s) and/or machine learning models to be executed in processing a request. In at least one embodiment, a request may then be passed to one or more components of deployment system 4306 (e.g., a cloud) to perform processing of data processing pipeline. In at least one embodiment, processing by deployment system 4306 may include referencing selected elements (e.g., applications, containers, models, etc.) from a container registry and/or model registry 4324. In at least one embodiment, once results are generated by a pipeline, results may be returned to a user for reference (e.g., for viewing in a viewing application suite executing on a local, on-premises workstation or terminal).

In at least one embodiment, to aid in processing or execution of applications or containers in pipelines, services 4320 may be leveraged. In at least one embodiment, services 4320 may include compute services, artificial intelligence (AI) services, visualization services, and/or other service types. In at least one embodiment, services 4320 may provide functionality that is common to one or more applications in software 4318, so functionality may be abstracted to a service that may be called upon or leveraged by applications. In at least one embodiment, functionality provided by services 4320 may run dynamically and more efficiently, while also scaling well by allowing applications to process data in parallel (e.g., using a parallel computing platform 4430 (FIG. 44)). In at least one embodiment, rather than each application that shares a same functionality offered by a service 4320 being required to have a respective instance of service 4320, service 4320 may be shared between and among various applications. In at least one embodiment, services may include an inference server or engine that may be used for executing detection or segmentation tasks, as non-limiting examples. In at least one embodiment, a model training service may be included that may provide machine learning model training and/or retraining capabilities.

In at least one embodiment, where a service 4320 includes an AI service (e.g., an inference service), one or more machine learning models associated with an application for anomaly detection (e.g., tumors, growth abnormalities, scarring, etc.) may be executed by calling upon (e.g., as an API call) an inference service (e.g., an inference server) to execute machine learning model(s), or processing thereof, as part of application execution. In at least one embodiment, where another application includes one or more machine learning models for segmentation tasks, an application may call upon an inference service to execute machine learning models for performing one or more of processing operations associated with segmentation tasks. In at least one embodiment, software 4318 implementing advanced processing and inferencing pipeline may be streamlined because each application may call upon a same inference service to perform one or more inferencing tasks.

In at least one embodiment, hardware 4322 may include GPUs, CPUs, graphics cards, an AI/deep learning system (e.g., an AI supercomputer, such as NVIDIA's DGX supercomputer system), a cloud platform, or a combination thereof. In at least one embodiment, different types of hardware 4322 may be used to provide efficient, purpose-built support for software 4318 and services 4320 in deployment system 4306. In at least one embodiment, use of GPU processing may be implemented for processing locally (e.g., at facility 4302), within an AI/deep learning system, in a cloud system, and/or in other processing components of deployment system 4306 to improve efficiency, accuracy, and efficacy of game name recognition.

In at least one embodiment, software 4318 and/or services 4320 may be optimized for GPU processing with respect to deep learning, machine learning, and/or high-performance computing, as non-limiting examples. In at least one embodiment, at least some of computing environment of deployment system 4306 and/or training system 4304 may be executed in a datacenter one or more supercomputers or high performance computing systems, with GPU optimized software (e.g., hardware and software combination of NVIDIA's DGX system). In at least one embodiment, hardware 4322 may include any number of GPUs that may be called upon to perform processing of data in parallel, as described herein. In at least one embodiment, cloud platform may further include GPU processing for GPU-optimized execution of deep learning tasks, machine learning tasks, or other computing tasks. In at least one embodiment, cloud platform (e.g., NVIDIA's NGC) may be executed using an AI/deep learning supercomputer(s) and/or GPU-optimized software (e.g., as provided on NVIDIA's DGX systems) as a hardware abstraction and scaling platform. In at least one embodiment, cloud platform may integrate an application container clustering system or orchestration system (e.g., KUBERNETES) on multiple GPUs to enable seamless scaling and load balancing.

FIG. 44 is a system diagram for an example system 4400 for generating and deploying a deployment pipeline, in accordance with at least one embodiment. In at least one embodiment, system 4400 may be used to implement process 4300 of FIG. 43 and/or other processes including advanced processing and inferencing pipelines. In at least one embodiment, system 4400 may include training system 4304 and deployment system 4306. In at least one embodiment, training system 4304 and deployment system 4306 may be implemented using software 4418, services 4420, and/or hardware 4422, as described herein.

In at least one embodiment, system 4400 (e.g., training system 4304 and/or deployment system 3606) may implemented in a cloud computing environment (e.g., using cloud 4426). In at least one embodiment, system 4400 may be implemented locally with respect to a facility, or as a combination of both cloud and local computing resources. In at least one embodiment, access to APIs in cloud 4426 may be restricted to authorized users through enacted security measures or protocols. In at least one embodiment, a security protocol may include web tokens that may be signed by an authentication (e.g., AuthN, AuthZ, Gluecon, etc.) service and may carry appropriate authorization. In at least one embodiment, APIs of virtual instruments (described herein), or other instantiations of system 4400, may be restricted to a set of public IPs that have been vetted or authorized for interaction.

In at least one embodiment, various components of system 4400 may communicate between and among one another using any of a variety of different network types, including but not limited to local area networks (LANs) and/or wide area networks (WANs) via wired and/or wireless communication protocols. In at least one embodiment, communication between facilities and components of system 4400 (e.g., for transmitting inference requests, for receiving results of inference requests, etc.) may be communicated over a data bus or data busses, wireless data protocols (Wi-Fi), wired data protocols (e.g., Ethernet), etc.

In at least one embodiment, training system 4304 may execute training pipelines 4404, similar to those described herein with respect to FIG. 43. In at least one embodiment, where one or more machine learning models are to be used in deployment pipelines 4410 by deployment system 4306, training pipelines 4404 may be used to train or retrain one or more (e.g., pre-trained) models, and/or implement one or more of pre-trained models 4306 (e.g., without a need for retraining or updating). In at least one embodiment, as a result of training pipelines 4404, output model(s) 4316 may be generated. In at least one embodiment, training pipelines 4404 may include any number of processing steps, AI-assisted annotation 4310, labeling or annotating of feedback data 4308 to generate labeled data 4312, model selection from a model registry, model training 4314, training, retraining, or updating models, and/or other processing steps. In at least one embodiment, for different machine learning models used by deployment system 4306, different training pipelines 4404 may be used. In at least one embodiment, training pipeline 4404 similar to a first example described with respect to FIG. 43 may be used for a first machine learning model, training pipeline 4404 similar to a second example described with respect to FIG. 43 may be used for a second machine learning model, and training pipeline 4404 similar to a third example described with respect to FIG. 43 may be used for a third machine learning model. In at least one embodiment, any combination of tasks within training system 4304 may be used depending on what is required for each respective machine learning model. In at least one embodiment, one or more of machine learning models may already be trained and ready for deployment so machine learning models may not undergo any processing by training system 4304, and may be implemented by deployment system 4306.

In at least one embodiment, output model(s) 4316 and/or pre-trained model(s) 4306 may include any types of machine learning models depending on implementation or embodiment. In at least one embodiment, and without limitation, machine learning models used by system 4400 may include machine learning model(s) using linear regression, logistic regression, decision trees, support vector machines (SVM), Naïve Bayes, k-nearest neighbor (Knn), K means clustering, random forest, dimensionality reduction algorithms, gradient boosting algorithms, neural networks (e.g., auto-encoders, convolutional, recurrent, perceptrons, Long/Short Term Memory (LSTM), Bi-LSTM, Hopfield, Boltzmann, deep belief, deconvolutional, generative adversarial, liquid state machine, etc.), and/or other types of machine learning models.

In at least one embodiment, training pipelines 4404 may include AI-assisted annotation. In at least one embodiment, labeled data 4312 (e.g., traditional annotation) may be generated by any number of techniques. In at least one embodiment, labels or other annotations may be generated within a drawing program (e.g., an annotation program), a computer aided design (CAD) program, a labeling program, another type of program suitable for generating annotations or labels for ground truth, and/or may be hand drawn, in some examples. In at least one embodiment, ground truth data may be synthetically produced (e.g., generated from computer models or renderings), real produced (e.g., designed and produced from real-world data), machine-automated (e.g., using feature analysis and learning to extract features from data and then generate labels), human annotated (e.g., labeler, or annotation expert, defines location of labels), and/or a combination thereof. In at least one embodiment, for each instance of feedback data 4308 (or other data type used by machine learning models), there may be corresponding ground truth data generated by training system 4304. In at least one embodiment, AI-assisted annotation may be performed as part of deployment pipelines 4410; either in addition to, or in lieu of AI-assisted annotation included in training pipelines 4404. In at least one embodiment, system 4400 may include a multi-layer platform that may include a software layer (e.g., software 4318) of diagnostic applications (or other application types) that may perform one or more medical imaging and diagnostic functions.

In at least one embodiment, a software layer may be implemented as a secure, encrypted, and/or authenticated API through which applications or containers may be invoked (e.g., called) from an external environment(s) (e.g., facility 4302). In at least one embodiment, applications may then call or execute one or more services 4320 for performing compute, AI, or visualization tasks associated with respective applications, and software 4318 and/or services 4320 may leverage hardware 4322 to perform processing tasks in an effective and efficient manner.

In at least one embodiment, deployment system 4306 may execute deployment pipelines 4410. In at least one embodiment, deployment pipelines 4410 may include any number of applications that may be sequentially, non-sequentially, or otherwise applied to feedback data (and/or other data types)—including AI-assisted annotation, as described above. In at least one embodiment, as described herein, a deployment pipeline 4410 for an individual device may be referred to as a virtual instrument for a device. In at least one embodiment, for a single device, there may be more than one deployment pipeline 4410 depending on information desired from data generated by a device.

In at least one embodiment, applications available for deployment pipelines 4410 may include any application that may be used for performing processing tasks on feedback data or other data from devices. In at least one embodiment, because various applications may share common image operations, a data augmentation library (e.g., as one of services 4320) may be used to accelerate these operations. In at least one embodiment, to avoid bottlenecks of conventional processing approaches that rely on CPU processing, parallel computing platform 4430 may be used for GPU acceleration of these processing tasks.

In at least one embodiment, deployment system 4306 may include a user interface 4414 (e.g., a graphical user interface, a web interface, etc.) that may be used to select applications for inclusion in deployment pipeline(s) 4410, arrange applications, modify or change applications or parameters or constructs thereof, use and interact with deployment pipeline(s) 4410 during set-up and/or deployment, and/or to otherwise interact with deployment system 4306. In at least one embodiment, although not illustrated with respect to training system 4304, user interface 4414 (or a different user interface) may be used for selecting models for use in deployment system 4306, for selecting models for training, or retraining, in training system 4304, and/or for otherwise interacting with training system 4304.

In at least one embodiment, pipeline manager 4412 may be used, in addition to an application orchestration system 4428, to manage interaction between applications or containers of deployment pipeline(s) 4410 and services 4320 and/or hardware 4322. In at least one embodiment, pipeline manager 4412 may be configured to facilitate interactions from application to application, from application to service 4320, and/or from application or service to hardware 4322. In at least one embodiment, although illustrated as included in software 4318, this is not intended to be limiting, and in some examples pipeline manager 4412 may be included in services 4320. In at least one embodiment, application orchestration system 4428 (e.g., Kubernetes, DOCKER, etc.) may include a container orchestration system that may group applications into containers as logical units for coordination, management, scaling, and deployment. In at least one embodiment, by associating applications from deployment pipeline(s) 4410 (e.g., a reconstruction application, a segmentation application, etc.) with individual containers, each application may execute in a self-contained environment (e.g., at a kernel level) to increase speed and efficiency.

In at least one embodiment, each application and/or container (or image thereof) may be individually developed, modified, and deployed (e.g., a first user or developer may develop, modify, and deploy a first application and a second user or developer may develop, modify, and deploy a second application separate from a first user or developer), which may allow for focus on, and attention to, a task of a single application and/or container(s) without being hindered by tasks of another application(s) or container(s). In at least one embodiment, communication, and cooperation between different containers or applications may be aided by pipeline manager 4412 and application orchestration system 4428. In at least one embodiment, so long as an expected input and/or output of each container or application is known by a system (e.g., based on constructs of applications or containers), application orchestration system 4428 and/or pipeline manager 4412 may facilitate communication among and between, and sharing of resources among and between, each of applications or containers. In at least one embodiment, because one or more of applications or containers in deployment pipeline(s) 4410 may share same services and resources, application orchestration system 4428 may orchestrate, load balance, and determine sharing of services or resources between and among various applications or containers. In at least one embodiment, a scheduler may be used to track resource requirements of applications or containers, current usage or planned usage of these resources, and resource availability. In at least one embodiment, a scheduler may thus allocate resources to different applications and distribute resources between and among applications in view of requirements and availability of a system. In some examples, a scheduler (and/or other component of application orchestration system 4428) may determine resource availability and distribution based on constraints imposed on a system (e.g., user constraints), such as quality of service (QoS), urgency of need for data outputs (e.g., to determine whether to execute real-time processing or delayed processing), etc.

In at least one embodiment, services 4320 leveraged by and shared by applications or containers in deployment system 4306 may include compute services 4416, AI services 4418, visualization services 4420, and/or other service types. In at least one embodiment, applications may call (e.g., execute) one or more of services 4320 to perform processing operations for an application. In at least one embodiment, compute services 4416 may be leveraged by applications to perform super-computing or other high-performance computing (HPC) tasks. In at least one embodiment, compute service(s) 4416 may be leveraged to perform parallel processing (e.g., using a parallel computing platform 4430) for processing data through one or more of applications and/or one or more tasks of a single application, substantially simultaneously. In at least one embodiment, parallel computing platform 4430 (e.g., NVIDIA's CUDA) may enable general purpose computing on GPUs (GPGPU) (e.g., GPUs 4422). In at least one embodiment, a software layer of parallel computing platform 4430 may provide access to virtual instruction sets and parallel computational elements of GPUs, for execution of compute kernels. In at least one embodiment, parallel computing platform 4430 may include memory and, in at least one embodiment, a memory may be shared between and among multiple containers, and/or between and among different processing tasks within a single container. In at least one embodiment, inter-process communication (IPC) calls may be generated for multiple containers and/or for multiple processes within a container to use same data from a shared segment of memory of parallel computing platform 4430 (e.g., where multiple different stages of an application or multiple applications are processing same information). In at least one embodiment, rather than making a copy of data and moving data to different locations in memory (e.g., a read/write operation), same data in same location of a memory may be used for any number of processing tasks (e.g., at a same time, at different times, etc.). In at least one embodiment, as data is used to generate new data as a result of processing, this information of a new location of data may be stored and shared between various applications. In at least one embodiment, location of data and a location of updated or modified data may be part of a definition of how a payload is understood within containers.

In at least one embodiment, AI services 4418 may be leveraged to perform inferencing services for executing machine learning model(s) associated with applications (e.g., tasked with performing one or more processing tasks of an application). In at least one embodiment, AI services 4418 may leverage AI system 4424 to execute machine learning model(s) (e.g., neural networks, such as CNNs) for segmentation, reconstruction, object detection, feature detection, classification, and/or other inferencing tasks. In at least one embodiment, applications of deployment pipeline(s) 4410 may use one or more of output models 4316 from training system 4304 and/or other models of applications to perform inference on imaging data (e.g., DICOM data, RIS data, CIS data, REST compliant data, RPC data, raw data, etc.). In at least one embodiment, two or more examples of inferencing using application orchestration system 4428 (e.g., a scheduler) may be available. In at least one embodiment, a first category may include a high priority/low latency path that may achieve higher service level agreements, such as for performing inference on urgent requests during an emergency, or for a radiologist during diagnosis. In at least one embodiment, a second category may include a standard priority path that may be used for requests that may be non-urgent or where analysis may be performed at a later time. In at least one embodiment, application orchestration system 4428 may distribute resources (e.g., services 4320 and/or hardware 4322) based on priority paths for different inferencing tasks of AI services 4418.

In at least one embodiment, shared storage may be mounted to AI services 4418 within system 4400. In at least one embodiment, shared storage may operate as a cache (or other storage device type) and may be used to process inference requests from applications. In at least one embodiment, when an inference request is submitted, a request may be received by a set of API instances of deployment system 4306, and one or more instances may be selected (e.g., for best fit, for load balancing, etc.) to process a request. In at least one embodiment, to process a request, a request may be entered into a database, a machine learning model may be located from model registry 4324 if not already in a cache, a validation step may ensure appropriate machine learning model is loaded into a cache (e.g., shared storage), and/or a copy of a model may be saved to a cache. In at least one embodiment, a scheduler (e.g., of pipeline manager 4412) may be used to launch an application that is referenced in a request if an application is not already running or if there are not enough instances of an application. In at least one embodiment, if an inference server is not already launched to execute a model, an inference server may be launched. In at least one embodiment, any number of inference servers may be launched per model. In at least one embodiment, in a pull model, in which inference servers are clustered, models may be cached whenever load balancing is advantageous. In at least one embodiment, inference servers may be statically loaded in corresponding, distributed servers.

In at least one embodiment, inferencing may be performed using an inference server that runs in a container. In at least one embodiment, an instance of an inference server may be associated with a model (and optionally a plurality of versions of a model). In at least one embodiment, if an instance of an inference server does not exist when a request to perform inference on a model is received, a new instance may be loaded. In at least one embodiment, when starting an inference server, a model may be passed to an inference server such that a same container may be used to serve different models so long as inference server is running as a different instance.

In at least one embodiment, during application execution, an inference request for a given application may be received, and a container (e.g., hosting an instance of an inference server) may be loaded (if not already), and a start procedure may be called. In at least one embodiment, pre-processing logic in a container may load, decode, and/or perform any additional pre-processing on incoming data (e.g., using a CPU(s) and/or GPU(s)). In at least one embodiment, once data is prepared for inference, a container may perform inference as necessary on data. In at least one embodiment, this may include a single inference call on one image (e.g., a hand X-ray), or may require inference on hundreds of images (e.g., a chest CT). In at least one embodiment, an application may summarize results before completing, which may include, without limitation, a single confidence score, pixel level-segmentation, voxel-level segmentation, generating a visualization, or generating text to summarize findings. In at least one embodiment, different models or applications may be assigned different priorities. For example, some models may have a real-time (TAT less than one minute) priority while others may have lower priority (e.g., TAT less than 10 minutes). In at least one embodiment, model execution times may be measured from requesting institution or entity and may include partner network traversal time, as well as execution on an inference service.

In at least one embodiment, transfer of requests between services 4320 and inference applications may be hidden behind a software development kit (SDK), and robust transport may be provide through a queue. In at least one embodiment, a request will be placed in a queue via an API for an individual application/tenant ID combination and an SDK will pull a request from a queue and give a request to an application. In at least one embodiment, a name of a queue may be provided in an environment from where an SDK will pick it up. In at least one embodiment, asynchronous communication through a queue may be useful as it may allow any instance of an application to pick up work as it becomes available. In at least one embodiment, results may be transferred back through a queue, to ensure no data is lost. In at least one embodiment, queues may also provide an ability to segment work, as highest priority work may go to a queue with most instances of an application connected to it, while lowest priority work may go to a queue with a single instance connected to it that processes tasks in an order received. In at least one embodiment, an application may run on a GPU-accelerated instance generated in cloud 4426, and an inference service may perform inferencing on a GPU.

In at least one embodiment, visualization services 4420 may be leveraged to generate visualizations for viewing outputs of applications and/or deployment pipeline(s) 4410. In at least one embodiment, GPUs 4422 may be leveraged by visualization services 4420 to generate visualizations. In at least one embodiment, rendering effects, such as ray-tracing, may be implemented by visualization services 4420 to generate higher quality visualizations. In at least one embodiment, visualizations may include, without limitation, 2D image renderings, 3D volume renderings, 3D volume reconstruction, 2D tomographic slices, virtual reality displays, augmented reality displays, etc. In at least one embodiment, virtualized environments may be used to generate a virtual interactive display or environment (e.g., a virtual environment) for interaction by users of a system (e.g., doctors, nurses, radiologists, etc.). In at least one embodiment, visualization services 4420 may include an internal visualizer, cinematics, and/or other rendering or image processing capabilities or functionality (e.g., ray tracing, rasterization, internal optics, etc.).

In at least one embodiment, hardware 4322 may include GPUs 4422, AI system 4424, cloud 4426, and/or any other hardware used for executing training system 4304 and/or deployment system 4306. In at least one embodiment, GPUs 4422 (e.g., NVIDIA's TESLA and/or QUADRO GPUs) may include any number of GPUs that may be used for executing processing tasks of compute services 4416, AI services 4418, visualization services 4420, other services, and/or any of features or functionality of software 4318. For example, with respect to AI services 4418, GPUs 4422 may be used to perform pre-processing on imaging data (or other data types used by machine learning models), post-processing on outputs of machine learning models, and/or to perform inferencing (e.g., to execute machine learning models). In at least one embodiment, cloud 4426, AI system 4424, and/or other components of system 4400 may use GPUs 4422. In at least one embodiment, cloud 4426 may include a GPU-optimized platform for deep learning tasks. In at least one embodiment, AI system 4424 may use GPUs, and cloud 4426—or at least a portion tasked with deep learning or inferencing—may be executed using one or more AI systems 4424. As such, although hardware 4322 is illustrated as discrete components, this is not intended to be limiting, and any components of hardware 4222 may be combined with, or leveraged by, any other components of hardware 4222.

In at least one embodiment, AI system 4424 may include a purpose-built computing system (e.g., a super-computer or an HPC) configured for inferencing, deep learning, machine learning, and/or other artificial intelligence tasks. In at least one embodiment, AI system 4424 (e.g., NVIDIA's DGX) may include GPU-optimized software (e.g., a software stack) that may be executed using a plurality of GPUs 4422, in addition to CPUs, RAM, storage, and/or other components, features, or functionality. In at least one embodiment, one or more AI systems 4424 may be implemented in cloud 4426 (e.g., in a data center) for performing some or all of AI-based processing tasks of system 4400.

In at least one embodiment, cloud 4426 may include a GPU-accelerated infrastructure (e.g., NVIDIA's NGC) that may provide a GPU-optimized platform for executing processing tasks of system 4400. In at least one embodiment, cloud 4426 may include an AI system(s) 4424 for performing one or more of AI-based tasks of system 4400 (e.g., as a hardware abstraction and scaling platform). In at least one embodiment, cloud 4426 may integrate with application orchestration system 4428 leveraging multiple GPUs to enable seamless scaling and load balancing between and among applications and services 4320. In at least one embodiment, cloud 4426 may tasked with executing at least some of services 4320 of system 4400, including compute services 4416, AI services 4418, and/or visualization services 4420, as described herein. In at least one embodiment, cloud 4426 may perform small and large batch inference (e.g., executing NVIDIA's TENSOR RT), provide an accelerated parallel computing API and platform 4430 (e.g., NVIDIA's CUDA), execute application orchestration system 4428 (e.g., KUBERNETES), provide a graphics rendering API and platform (e.g., for ray-tracing, 2D graphics, 3D graphics, and/or other rendering techniques to produce higher quality cinematics), and/or may provide other functionality for system 4400.

In at least one embodiment, in an effort to preserve patient confidentiality (e.g., where patient data or records are to be used off-premises), cloud 4426 may include a registry—such as a deep learning container registry. In at least one embodiment, a registry may store containers for instantiations of applications that may perform pre-processing, post-processing, or other processing tasks on patient data. In at least one embodiment, cloud 4426 may receive data that includes patient data as well as sensor data in containers, perform requested processing for just sensor data in those containers, and then forward a resultant output and/or visualizations to appropriate parties and/or devices (e.g., on-premises medical devices used for visualization or diagnoses), all without having to extract, store, or otherwise access patient data. In at least one embodiment, confidentiality of patient data is preserved in compliance with HIPAA and/or other data regulations.

At least one embodiment of the disclosure can be described in view of the following clauses:

In clause 1, a processor comprises: one or more circuits to identify one or more relationships among one or more words using one or more transformer-based language neural networks trained using domain-specific data.

In clause 2, a processor of clause 1, wherein one or more relationships among one or more words comprises a score indicating a quantified relationship between a query phrase of one or more words and a target phrase of one or more words, wherein score is a positive number or a negative number.

In clause 3, a processor of clause 1, wherein one or more transformer-based language neural networks comprise: a query-target conditioning layer, using a softmax function, to compute a conditional probability for each target word in a target phrase given a query word in a query phrase; and a summation layer to sum conditional probability for each target word in target phrase to obtain a score indicating a quantified relationship between query word and target phrase.

In clause 4, a processor of clause 3 wherein one or more transformer-based language neural networks comprise a layer to: compute a first masked language prediction for a query word; compute a second masked language prediction for each target word in a target phrase; perform a dot product multiplication of first masked language prediction and second masked language prediction to obtain query-target predictions for target phrase; and sum query-target predictions for target phrase to obtain a score indicating a quantified relationship between query word and target phrase.

In clause 5, a processor of clause 4, wherein query-target predictions comprises a first query-target prediction that is a positive number indicating a positive relationship between query word and a corresponding target word in target phrase.

In clause 5, a processor of clause 4, wherein query-target predictions comprise a first query-target prediction that is a negative number indicating a negative relationship between query word and a corresponding target word in target phrase.

In clause 6, a processor of clause 1, wherein one or more words comprise a query phrase of one or more words and a target phrase of one or more words, and wherein one or more transformer-based language neural networks comprise: a scoring function to sum and normalize a score of an association between each word of query phrase and each word of target phrase; and a ranking function to rank an item of interest in query phrase for a desirable property in target phrase.

In clause 7, a processor of clause 1, wherein one or more words comprise a query phrase of one or more words and a target phrase of one or more words, and wherein one or more transformer-based language neural networks comprise: a scoring function to sum and normalize a score of an association between each word of query phrase and each word of target phrase; and a ranking function to rank an item of interest in query phrase for a desirable property in target phrase.

In clause 8, a processor of clause 1, wherein one or more transformer-based language neural networks are trained with domain-specific data using a Robustly Optimized Bidirectional Encoder Representations from Transformers approach (RoBERTa).

In clause 9, a processor of clause 1, wherein one or more transformer-based language neural networks comprises: an input layer to: receive additional domain-specific data during an inference phase; receive a query phrase of one or more words and encode query phrase into a first vector of tokens using byte-pair encoding (BPE); receive a target phrase of one or more words and encode target phrase into a second vector of tokens using BPE; a Bidirectional Encoder Representations from Transformers (BERT) layer that is trained using a Robustly Optimized Bidirectional Encoder Representations from Transformers Approach (RoBERTa) and comprises: a first attention head to receive first vector of tokens and compute a statistical prediction for each token in first vector of tokens; a second attention head to receive second vector of tokens and compute a statistical prediction for each token in second vector of tokens; and an output layer to determine a query-target score by performing a dot product multiplication on statistical predictions of first vector of tokens and statistical prediction of second vector of tokens.

In clause 10, a processor comprises: one or more circuits to use one or more transformer-based language neural networks to identify one or more drugs described in one or more documents, wherein transformer-based language neural network is trained using domain-specific data.

In clause 11, a processor of clause 10, wherein one or more circuits are to use one or more transformer-based language neural networks to rank one or more drug candidates from a clinical trials dataset based, at least in part, on conditional probabilities for an association between each of drug candidates and at least one target property.

In clause 12, a processor of clause 10, wherein one or more circuits are to use one or more transformer-based language neural networks to determine a drug candidate for drug approval by ranking drug candidates from a clinical trials dataset based, at least in part, on query-target conditioning predictions of drug candidates as query words in clinical trials dataset and an efficacy property as a target in clinical trials dataset.

In clause 13, a processor of clause 10, wherein one or more circuits, to identify one or more drugs, are further to: for a set of drug candidate from a clinical trials dataset, compute a conditional probability for each target word in a target phrase given a query word corresponding to respective drug candidate, and sum conditional probabilities for target phrase given query word to obtain a score for respective drug candidate; and rank drug candidates according to score, wherein the one or more transformer-based language neural networks are trained with the domain-specific data using a Robustly Optimized Bidirectional Encoder Representations from Transformers approach (RoBERTa).

In clause 14, a processor of clause 10, wherein one or more transformer-based language neural networks comprises: an input layer to: receive a clinical trials dataset for a set of drugs; for each drug of set of drugs, receive a query word corresponding to respective drug and encode query word into a first vector using byte-pair encoding (BPE); receive a target phrase of one or more words and encode target phrase into a second vector of tokens using BPE, wherein target phrase comprises a target property of efficacy; a Bidirectional Encoder Representations from Transformers (BERT) layer that is trained using a Robustly Optimized Bidirectional Encoder Representations from Transformers Approach (RoBERTa) and determines a drug score for each drug of set of drugs; and an output layer to rank set of drugs according to drug scores.

In clause 15, a system comprises: one or more processors to use one or more transformer-based language neural networks trained using domain-specific data to identify one or more relationships among one or more words; and one or more memories to store parameters associated with one or more transformer-based language neural networks.

In clause 16, a system of clause 15, wherein one or more relationships among one or more words comprises a score indicating a quantified relationship between a query phrase of one or more words and a target phrase of one or more words, wherein score is a positive number of a negative number.

In clause 17, a system of clause 15, wherein one or more transformer-based language neural networks comprise: a query-target conditioning layer, using a softmax function, to compute a conditional probability for each target word in a target phrase given a query word in a query phrase; and a summation layer to sum conditional probability for each target word in target phrase to obtain a score indicating a quantified relationship between query word and target phrase.

In clause 18, a system of clause 15, wherein one or more transformer-based language neural networks comprise a layer to: compute a first masked language prediction for a query word; compute a second masked language prediction for each target word in a target phrase; perform a dot product multiplication of first masked language prediction and second masked language prediction to filter first masked language prediction and second masked language prediction to obtain query-target predictions for target phrase; and sum query-target predictions for target phrase to obtain a score indicating a quantified relationship between query word and target phrase.

In clause 19, a system of clause 18, wherein query-target predictions comprises a first query-target prediction that is a positive number indicating a positive relationship between query word and a corresponding target word in target phrase.

In clause 20, a system of clause 18, wherein query-target predictions comprises a first query-target prediction that is a negative number indicating a negative relationship between query word and a corresponding target word in target phrase.

In clause 21, a system of clause 15, wherein one or more transformer-based language neural networks are trained with domain-specific data using a Robustly Optimized Bidirectional Encoder Representations from Transformers approach (RoBERTa).

In clause 22, a system of clause 15, wherein one or more transformer-based language neural networks comprises: an input layer to: receive additional domain-specific data during an inference phase; receive a query phrase of one or more words and encode query phrase into a first vector of tokens using byte-pair encoding (BPE); receive a target phrase of one or more words and encode target phrase into a second vector of tokens using BPE; a Bidirectional Encoder Representations from Transformers (BERT) layer that is trained using a Robustly Optimized Bidirectional Encoder Representations from Transformers Approach (RoBERTa) and comprises: a first attention head to receive first vector of tokens and compute a statistical prediction for each token in first vector of tokens; a second attention head to receive second vector of tokens and compute a statistical prediction for each token in second vector of tokens; and an output layer to determine a query-target score by performing a dot product multiplication on statistical predictions of first vector of tokens and statistical prediction of second vector of tokens.

In clause 23, a system comprising: one or more processors to use one or more transformer-based language neural networks to identify one or more drugs described in one or more documents, wherein transformer-based language neural network is trained using domain-specific data; and one or more memories to store parameters associated with transformer-based language neural networks.

In clause 24, a system of clause 23, wherein one or more circuits are to use one or more transformer-based language neural networks to rank one or more drug candidates from a clinical trials dataset based, at least in part, on conditional probabilities for an association between each of drug candidates and at least one target property.

In clause 25, a system of clause 23, wherein one or more transformer-based language neural networks comprises: an input layer to: receive a clinical trials dataset for a set of drugs; for each drug of set of drugs, receive a query word corresponding to respective drug and encode query word into a first vector using byte-pair encoding (BPE); receive a target phrase of one or more words and encode target phrase into a second vector of tokens using BPE, wherein target phrase comprises a target property of efficacy; a Bidirectional Encoder Representations from Transformers (BERT) layer that is trained using a Robustly Optimized Bidirectional Encoder Representations from Transformers Approach (RoBERTa) and determines a drug score for each drug of set of drugs; and an output layer to rank set of drugs according to drug scores.

In clause 26, a machine-readable medium having stored thereon a set of instructions, which if performed by one or more processors, cause one or more processors to at least identify one or more relationships among one or more words using one or more transformer-based language neural networks trained using domain-specific data.

In clause 27, a machine-readable medium of clause 26, wherein one or more relationships among one or more words comprises a score indicating a quantified relationship between a query phrase of one or more words and a target phrase of one or more words, wherein score is a positive number or a negative number.

In clause 28, a machine-readable medium of clause 26, wherein one or more transformer-based language neural networks comprises: an input layer to: receive additional domain-specific data during an inference phase; receive a query phrase of one or more words and encode query phrase into a first vector of tokens using byte-pair encoding (BPE); receive a target phrase of one or more words and encode target phrase into a second vector of tokens using BPE; a Bidirectional Encoder Representations from Transformers (BERT) layer that is trained using a Robustly Optimized Bidirectional Encoder Representations from Transformers Approach (RoBERTa) and comprises: a first attention head to receive first vector of tokens and compute a statistical prediction for each token in first vector of tokens; a second attention head to receive second vector of tokens and compute a statistical prediction for each token in second vector of tokens; and an output layer to determine a query-target score by performing a dot product multiplication on statistical predictions of first vector of tokens and statistical prediction of second vector of tokens.

In clause 29, a machine-readable medium having stored thereon a set of instructions, which if performed by one or more processors, cause one or more processors to at least identify one or more drugs described in one or more documents using one or more transformer-based language neural networks, wherein transformer-based language neural network is trained using domain-specific data.

In clause 30, a machine-readable medium of clause 29, wherein one or more circuits are to use one or more transformer-based language neural networks to rank one or more drug candidates from a clinical trials dataset based, at least in part, on conditional probabilities for an association between each of drug candidates and at least one target property.

In clause 31, a machine-readable medium of clause 29, wherein one or more transformer-based language neural networks comprises: an input layer to: receive additional domain-specific data during an inference phase; receive a query phrase of one or more words and encode query phrase into a first vector of tokens using byte-pair encoding (BPE); receive a target phrase of one or more words and encode target phrase into a second vector of tokens using BPE; a Bidirectional Encoder Representations from Transformers (BERT) layer that is trained using a Robustly Optimized Bidirectional Encoder Representations from Transformers Approach (RoBERTa) and comprises: a first attention head to receive first vector of tokens and compute a statistical prediction for each token in first vector of tokens; a second attention head to receive second vector of tokens and compute a statistical prediction for each token in second vector of tokens; and an output layer to determine a query-target score by performing a dot product multiplication on statistical predictions of first vector of tokens and statistical prediction of second vector of tokens.

In clause 32, a method comprises: receiving one or more input words; and identifying, using one or more transformer-based language neural networks, one or more relationships among one or more input words.

In clause 33, a method comprises: receiving one or more input words for one or more transformer-based language neural networks that are trained on domain-specific data; and identifying, using one or more transformer-based language network networks, one or more drugs in one or more documents.

In at least one embodiment, a single semiconductor platform may refer to a sole unitary semiconductor-based integrated circuit or chip. In at least one embodiment, multi-chip modules may be used with increased connectivity which simulate on-chip operation, and make substantial improvements over utilizing a conventional central processing unit (“CPU”) and bus implementation. In at least one embodiment, various modules may also be situated separately or in various combinations of semiconductor platforms per desires of user.

In at least one embodiment, referring back to FIG. 20, computer programs in form of machine-readable executable code or computer control logic algorithms are stored in main memory 2004 and/or secondary storage. Computer programs, if executed by one or more processors, enable system 2000 to perform various functions in accordance with at least one embodiment. In at least one embodiment, memory 2004, storage, and/or any other storage are possible examples of computer-readable media. In at least one embodiment, secondary storage may refer to any suitable storage device or system such as a hard disk drive and/or a removable storage drive, representing a floppy disk drive, a magnetic tape drive, a compact disk drive, digital versatile disk (“DVD”) drive, recording device, universal serial bus (“USB”) flash memory, etc. In at least one embodiment, architecture and/or functionality of various previous figures are implemented in context of CPU 2002, parallel processing system 2012, an integrated circuit capable of at least a portion of capabilities of both CPU 2002, parallel processing system 2012, a chipset (e.g., a group of integrated circuits designed to work and sold as a unit for performing related functions, etc.), and/or any suitable combination of integrated circuit(s).

In at least one embodiment, architecture and/or functionality of various previous figures are implemented in context of a general computer system, a circuit board system, a game console system dedicated for entertainment purposes, an application-specific system, and more. In at least one embodiment, computer system 2000 may take form of a desktop computer, a laptop computer, a tablet computer, servers, supercomputers, a smart-phone (e.g., a wireless, hand-held device), personal digital assistant (“PDA”), a digital camera, a vehicle, a head mounted display, a hand-held electronic device, a mobile phone device, a television, workstation, game consoles, embedded system, and/or any other type of logic.

In at least one embodiment, parallel processing system 2012 includes, without limitation, a plurality of parallel processing units (“PPUs”) 2014 and associated memories 2016. In at least one embodiment, PPUs 2014 are connected to a host processor or other peripheral devices via an interconnect 2018 and a switch 2020 or multiplexer. In at least one embodiment, parallel processing system 2012 distributes computational tasks across PPUs 2014 which can be parallelizable—for example, as part of distribution of computational tasks across multiple graphics processing unit (“GPU”) thread blocks. In at least one embodiment, memory is shared and accessible (e.g., for read and/or write access) across some or all of PPUs 2014, although such shared memory may incur performance penalties relative to use of local memory and registers resident to a PPU 2014. In at least one embodiment, operation of PPUs 2014 is synchronized through use of a command such as _syncthreads( ), wherein all threads in a block (e.g., executed across multiple PPUs 2014) to reach a certain point of execution of code before proceeding.

Other variations are within spirit of present disclosure. Thus, while disclosed techniques are susceptible to various modifications and alternative constructions, certain illustrated embodiments thereof are shown in drawings and have been described above in detail. It should be understood, however, that there is no intention to limit disclosure to specific form or forms disclosed, but on contrary, intention is to cover all modifications, alternative constructions, and equivalents falling within spirit and scope of disclosure, as defined in appended claims.

Use of terms “a” and “an” and “the” and similar referents in context of describing disclosed embodiments (especially in context of following claims) are to be construed to cover both singular and plural, unless otherwise indicated herein or clearly contradicted by context, and not as a definition of a term. Terms “comprising,” “having,” “including,” and “containing” are to be construed as open-ended terms (meaning “including, but not limited to,”) unless otherwise noted. “Connected,” when unmodified and referring to physical connections, is to be construed as partly or wholly contained within, attached to, or joined together, even if there is something intervening. Recitation of ranges of values herein are merely intended to serve as a shorthand method of referring individually to each separate value falling within range, unless otherwise indicated herein and each separate value is incorporated into specification as if it were individually recited herein. In at least one embodiment, use of term “set” (e.g., “a set of items”) or “subset” unless otherwise noted or contradicted by context, is to be construed as a nonempty collection comprising one or more members. Further, unless otherwise noted or contradicted by context, term “subset” of a corresponding set does not necessarily denote a proper subset of corresponding set, but subset and corresponding set may be equal.

Conjunctive language, such as phrases of form “at least one of A, B, and C,” or “at least one of A, B and C,” unless specifically stated otherwise or otherwise clearly contradicted by context, is otherwise understood with context as used in general to present that an item, term, etc., may be either A or B or C, or any nonempty subset of set of A and B and C. For instance, in illustrative example of a set having three members, conjunctive phrases “at least one of A, B, and C” and “at least one of A, B and C” refer to any of following sets: {A}, {B}, {C}, {A, B}, {A, C}, {B, C}, {A, B, C}. Thus, such conjunctive language is not generally intended to imply that certain embodiments require at least one of A, at least one of B and at least one of C each to be present. In addition, unless otherwise noted or contradicted by context, term “plurality” indicates a state of being plural (e.g., “a plurality of items” indicates multiple items). In at least one embodiment, number of items in a plurality is at least two, but can be more when so indicated either explicitly or by context. Further, unless stated otherwise or otherwise clear from context, phrase “based on” means “based at least in part on” and not “based solely on.”

Operations of processes described herein can be performed in any suitable order unless otherwise indicated herein or otherwise clearly contradicted by context. In at least one embodiment, a process such as those processes described herein (or variations and/or combinations thereof) is performed under control of one or more computer systems configured with executable instructions and is implemented as code (e.g., executable instructions, one or more computer programs or one or more applications) executing collectively on one or more processors, by hardware or combinations thereof. In at least one embodiment, code is stored on a computer-readable storage medium, for example, in form of a computer program comprising a plurality of instructions executable by one or more processors. In at least one embodiment, a computer-readable storage medium is a non-transitory computer-readable storage medium that excludes transitory signals (e.g., a propagating transient electric or electromagnetic transmission) but includes non-transitory data storage circuitry (e.g., buffers, cache, and queues) within transceivers of transitory signals. In at least one embodiment, code (e.g., executable code or source code) is stored on a set of one or more non-transitory computer-readable storage media having stored thereon executable instructions (or other memory to store executable instructions) that, when executed (i.e., as a result of being executed) by one or more processors of a computer system, cause computer system to perform operations described herein. In at least one embodiment, set of non-transitory computer-readable storage media comprises multiple non-transitory computer-readable storage media and one or more of individual non-transitory storage media of multiple non-transitory computer-readable storage media lack all of code while multiple non-transitory computer-readable storage media collectively store all of code. In at least one embodiment, executable instructions are executed such that different instructions are executed by different processors—for example, a non-transitory computer-readable storage medium store instructions and a main central processing unit (“CPU”) executes some of instructions while a graphics processing unit (“GPU”) executes other instructions. In at least one embodiment, different components of a computer system have separate processors and different processors execute different subsets of instructions.

Accordingly, in at least one embodiment, computer systems are configured to implement one or more services that singly or collectively perform operations of processes described herein and such computer systems are configured with applicable hardware and/or software that enable performance of operations. Further, a computer system that implements at least one embodiment of present disclosure is a single device and, in at least one other embodiment, is a distributed computer system comprising multiple devices that operate differently such that distributed computer system performs operations described herein and such that a single device does not perform all operations.

Use of any and all examples, or exemplary language (e.g., “such as”) provided herein, is intended merely to better illuminate embodiments of disclosure and does not pose a limitation on scope of disclosure unless otherwise claimed. No language in specification should be construed as indicating any non-claimed element as essential to practice of disclosure.

All references, including publications, patent applications, and patents, cited herein are hereby incorporated by reference to same extent as if each reference were individually and specifically indicated to be incorporated by reference and were set forth in its entirety herein.

In description and claims, terms “coupled” and “connected,” along with their derivatives, may be used. It should be understood that these terms may be not intended as synonyms for each other. Rather, in particular examples, “connected” or “coupled” may be used to indicate that two or more elements are in direct or indirect physical or electrical contact with each other. “Coupled” may also mean that two or more elements are not in direct contact with each other, but yet still co-operate or interact with each other.

Unless specifically stated otherwise, it may be appreciated that throughout specification terms such as “processing,” “computing,” “calculating,” “determining,” or like, refer to action and/or processes of a computer or computing system, or similar electronic computing device, that manipulate and/or transform data represented as physical, such as electronic, quantities within computing system's registers and/or memories into other data similarly represented as physical quantities within computing system's memories, registers or other such information storage, transmission or display devices.

In a similar manner, term “processor” may refer to any device or portion of a device that processes electronic data from registers and/or memory and transform that electronic data into other electronic data that may be stored in registers and/or memory. As non-limiting examples, “processor” may be a CPU or a GPU. A “computing platform” may comprise one or more processors. As used herein, “software” processes may include, for example, software and/or hardware entities that perform work over time, such as tasks, threads, and intelligent agents. Also, each process may refer to multiple processes, for carrying out instructions in sequence or in parallel, continuously or intermittently. In at least one embodiment, terms “system” and “method” are used herein interchangeably insofar as system may embody one or more methods and methods may be considered a system.

In present document, references may be made to obtaining, acquiring, receiving, or inputting analog or digital data into a subsystem, computer system, or computer-implemented machine. In at least one embodiment, process of obtaining, acquiring, receiving, or inputting analog and digital data can be accomplished in a variety of ways such as by receiving data as a parameter of a function call or a call to an application programming interface. In at least one embodiment, processes of obtaining, acquiring, receiving, or inputting analog or digital data can be accomplished by transferring data via a serial or parallel interface. In at least one embodiment, processes of obtaining, acquiring, receiving, or inputting analog or digital data can be accomplished by transferring data via a computer network from providing entity to acquiring entity. In at least one embodiment, references may also be made to providing, outputting, transmitting, sending, or presenting analog or digital data. In various examples, processes of providing, outputting, transmitting, sending, or presenting analog or digital data can be accomplished by transferring data as an input or output parameter of a function call, a parameter of an application programming interface or interprocess communication mechanism.

Although descriptions herein set forth example implementations of described techniques, other architectures may be used to implement described functionality, and are intended to be within scope of this disclosure. Furthermore, although specific distributions of responsibilities may be defined above for purposes of description, various functions and responsibilities might be distributed and divided in different ways, depending on circumstances.

Furthermore, although subject matter has been described in language specific to structural features and/or methodological acts, it is to be understood that subject matter claimed in appended claims is not necessarily limited to specific features or acts described. Rather, specific features and acts are disclosed as exemplary forms of implementing claims.

What is claimed is:

1. A processor comprising: one or more circuits to identify one or more relationships among one or more words using one or more transformer-based language neural networks trained using domain-specific data.

2. The processor of claim 1, wherein the one or more relationships among one or more words comprises a score indicating a quantified relationship between a query phrase of one or more words and a target phrase of one or more words, wherein the score is a positive number or a negative number.

3. The processor of claim 1, wherein the one or more transformer-based language neural networks comprise:
a query-target conditioning layer, using a softmax function, to compute a conditional probability for each target word in a target phrase given a query word in a query phrase; and
a summation layer to sum the conditional probability for each target word in the target phrase to obtain a score indicating a quantified relationship between the query word and the target phrase.

4. The processor of claim 1, wherein the one or more transformer-based language neural networks comprise a layer to:
compute a first masked language prediction for a query word;
compute a second masked language prediction for each target word in a target phrase;
perform a dot product multiplication of the first masked language prediction and the second masked language prediction to obtain query-target predictions for the target phrase; and
sum the query-target predictions for the target phrase to obtain a score indicating a quantified relationship between the query word and the target phrase.

5. The processor of claim 4, wherein the query-target predictions comprises a first query-target prediction that is a positive number indicating a positive relationship between the query word and a corresponding target word in the target phrase.

6. The processor of claim 4, wherein the query-target predictions comprise a first query-target prediction that is a negative number indicating a negative relationship between the query word and a corresponding target word in the target phrase.

7. The processor of claim 1, wherein the one or more words comprise a query phrase of one or more words and a target phrase of one or more words, and wherein the one or more transformer-based language neural networks comprise:
a scoring function to sum and normalize a score of an association between each word of the query phrase and each word of the target phrase; and
a ranking function to rank an item of interest in the query phrase for a desirable property in the target phrase.

8. The processor of claim 1, wherein the one or more transformer-based language neural networks are trained with the domain-specific data using a Robustly Optimized Bidirectional Encoder Representations from Transformers approach (RoBERTa).

9. The processor of claim 1, wherein the one or more transformer-based language neural networks comprises:
an input layer to:
receive additional domain-specific data during an inference phase;
receive a query phrase of one or more words and encode the query phrase into a first vector of tokens using byte-pair encoding (BPE);
receive a target phrase of one or more words and encode the target phrase into a second vector of tokens using BPE;
a Bidirectional Encoder Representations from Transformers (BERT) layer that is trained using a Robustly Optimized Bidirectional Encoder Representations from Transformers Approach (RoBERTa) and comprises:
a first attention head to receive the first vector of tokens and compute a statistical prediction for each token in the first vector of tokens;
a second attention head to receive the second vector of tokens and compute a statistical prediction for each token in the second vector of tokens; and
an output layer to determine a query-target score by performing a dot product multiplication on the statistical predictions of the first vector of tokens and the statistical prediction of the second vector of tokens.

10. A processor comprising: one or more circuits to use one or more transformer-based language neural networks to identify one or more drugs described in one or more documents, wherein the transformer-based language neural network is trained using domain-specific data.

11. The processor of claim 10, wherein the one or more circuits are to use the one or more transformer-based language neural networks to rank one or more drug candidates from a clinical trials dataset based, at least in part, on conditional probabilities for an association between each of the drug candidates and at least one target property.

12. The processor of claim 10, wherein the one or more circuits are to use the one or more transformer-based language neural networks to determine a drug candidate for drug approval by ranking drug candidates from a clinical trials dataset based, at least in part, on query-target conditioning predictions of the drug candidates as query words in the clinical trials dataset and an efficacy property as a target in the clinical trials dataset.

13. The processor of claim 10, wherein the one or more circuits, to identify the one or more drugs, are further to:
for a set of drug candidate from a clinical trials dataset,
compute a conditional probability for each target word in a target phrase given a query word corresponding to the respective drug candidate, and
sum the conditional probabilities for the target phrase given the query word to obtain a score for the respective drug candidate; and
rank the drug candidates according to the score, wherein the one or more transformer-based language neural networks are trained with the domain-specific data using a Robustly Optimized Bidirectional Encoder Representations from Transformers approach (RoBERTa).

14. The processor of claim 10, wherein the one or more transformer-based language neural networks comprises:
an input layer to:
receive a clinical trials dataset for a set of drugs;
for each drug of the set of drugs, receive a query word corresponding to the respective drug and encode the query word into a first vector using byte-pair encoding (BPE);
receive a target phrase of one or more words and encode the target phrase into a second vector of tokens using BPE, wherein the target phrase comprises a target property of efficacy;
a Bidirectional Encoder Representations from Transformers (BERT) layer that is trained using a Robustly Optimized Bidirectional Encoder Representations from Transformers Approach (RoBERTa) and determines a drug score for each drug of the set of drugs; and
an output layer to rank the set of drugs according to drug scores.

15. A system comprising:
one or more processors to use one or more transformer-based language neural networks trained using domain-specific data to identify one or more relationships among one or more words; and
one or more memories to store parameters associated with the one or more transformer-based language neural networks.

16. The system of claim 15, wherein the one or more relationships among the one or more words comprises a score indicating a quantified relationship between a query phrase of one or more words and a target phrase of one or more words, wherein the score is a positive number of a negative number.

17. The system of claim 15, wherein the one or more transformer-based language neural networks comprise:
a query-target conditioning layer, using a softmax function, to compute a conditional probability for each target word in a target phrase given a query word in a query phrase; and
a summation layer to sum the conditional probability for each target word in the target phrase to obtain a score indicating a quantified relationship between the query word and the target phrase.

18. The system of claim 15, wherein the one or more transformer-based language neural networks comprise a layer to:
compute a first masked language prediction for a query word;
compute a second masked language prediction for each target word in a target phrase;
perform a dot product multiplication of the first masked language prediction and the second masked language prediction to filter the first masked language prediction and the second masked language prediction to obtain query-target predictions for the target phrase; and
sum the query-target predictions for the target phrase to obtain a score indicating a quantified relationship between the query word and the target phrase.

19. The system of claim 18, wherein the query-target predictions comprises a first query-target prediction that is a positive number indicating a positive relationship between the query word and a corresponding target word in the target phrase.

20. The system of claim 18, wherein the query-target predictions comprises a first query-target prediction that is a negative number indicating a negative relationship between the query word and a corresponding target word in the target phrase.

21. The system of claim 15, wherein the one or more transformer-based language neural networks are trained with the domain-specific data using a Robustly Optimized Bidirectional Encoder Representations from Transformers approach (RoBERTa).

22. The system of claim 15, wherein the one or more transformer-based language neural networks comprises:
an input layer to:
receive additional domain-specific data during an inference phase;
receive a query phrase of one or more words and encode the query phrase into a first vector of tokens using byte-pair encoding (BPE);
receive a target phrase of one or more words and encode the target phrase into a second vector of tokens using BPE;
a Bidirectional Encoder Representations from Transformers (BERT) layer that is trained using a Robustly Optimized Bidirectional Encoder Representations from Transformers Approach (RoBERTa) and comprises:
a first attention head to receive the first vector of tokens and compute a statistical prediction for each token in the first vector of tokens;
a second attention head to receive the second vector of tokens and compute a statistical prediction for each token in the second vector of tokens; and
an output layer to determine a query-target score by performing a dot product multiplication on the statistical predictions of the first vector of tokens and the statistical prediction of the second vector of tokens.

23. A system comprising:
one or more processors to use one or more transformer-based language neural networks to identify one or more drugs described in one or more documents, wherein the transformer-based language neural network is trained using domain-specific data; and
one or more memories to store parameters associated with the transformer-based language neural networks.

24. The system of claim 23, wherein the one or more circuits are to use the one or more transformer-based language neural networks to rank one or more drug candidates from a clinical trials dataset based, at least in part, on conditional probabilities for an association between each of the drug candidates and at least one target property.

25. The system of claim 23, wherein the one or more transformer-based language neural networks comprises:
an input layer to:
receive a clinical trials dataset for a set of drugs;
for each drug of the set of drugs, receive a query word corresponding to the respective drug and encode the query word into a first vector using byte-pair encoding (BPE);
receive a target phrase of one or more words and encode the target phrase into a second vector of tokens using BPE, wherein the target phrase comprises a target property of efficacy;
a Bidirectional Encoder Representations from Transformers (BERT) layer that is trained using a Robustly Optimized Bidirectional Encoder Representations from Transformers Approach (RoBERTa) and determines a drug score for each drug of the set of drugs; and
an output layer to rank the set of drugs according to the drug scores.
